(19) United States
(12) Patent Application Publication
(10) Pub. No.: US 2025/0340847 A1
(43) Pub. Date: Nov. 6, 2025

(54) CELL CULTURE SYSTEM, USES THEREOF AND CELLS DERIVED THEREFROM

(71) Applicant: UNIVERSITY OF PITTSBURGH-OF THE COMMONWEALTH SYSTEM OF HIGHER EDUCATION, Pittsburgh, PA (US)

(72) Inventors: Rayna SCHOENBERGER, Pittsburgh, PA (US); Joshua Jamie HISLOP, Pittsburgh, PA (US); Mo Reza EBRAHIMKHANI, Pittsburgh, PA (US); Samira KIANI, Pittsburgh, PA (US)

(21) Appl. No.: 18/868,974

(22) PCT Filed: May 24, 2023
(86) PCT No.: PCT/US2023/067404

§ 371 Date: Nov. 25, 2024

Related U.S. Application Data
(60) Provisional application No. 63/345,233, filed on May 24, 2022.

(51) Int. Cl. C12N 5/074; C12N 5/0789

(52) U.S. Cl. CPC C12N 5/0696; C12N 5/0647; C12N 2501/60; C12N 2510/00

(57) ABSTRACT

The present disclosure is related to a cell culture system and uses thereof for generating a cellular mass. The cellular mass described herein can be used for studying embryogenesis and hematopoiesis and for treating diseases.

### CROSS-REFERENCE TO RELATED APPLICATIONS

This application is a U.S. National Stage Application filed under 35 U.S.C. § 371 of PCT/US2023/067404, filed on May 24, 2023, which claims the benefit of U.S. Provisional Application No. 63/345,233, filed May 24, 2022, which are expressly incorporated herein by reference in their entirety.

### STATEMENT REGARDING FEDERALLY SPONSORED RESEARCH OR DEVELOPMENT

This invention was made with Government support under grant numbers HL141805, DK120531, and EB001026 awarded by the National Institutes of Health and grant number 2134999 awarded by the National Science Foundation. The U.S. Government has certain rights in this invention.

### REFERENCE TO SEQUENCE LISTING

A Sequence Listing conforming to the rules of WIPO Standard ST.26 is hereby incorporated by reference. Said Sequence Listing has been filed as an electronic document via Patent Center in ASCII format encoded as XML. The electronic document, created on May 24, 2023, is entitled “10504-082WO1.xml”, and is 50,105 bytes in size.

### FIELD

The present disclosure relates to in vitro and ex vivo culture systems for generating a cellular mass that comprises one or more embryonic cell structures and one or more extraembryonic cell structures.

### BACKGROUND

Decoding mechanisms of human embryogenesis have tremendous biomedical impact, from treating congenital diseases and infertility, to engineering functional human organs. Immediately post implantation, the embryo and co-developing extra-embryonic tissues are profoundly remodeled and initiate morphological changes central for the success of pregnancy including formation of amniotic cavity or emergence of yolk sac hematopoiesis (Muller, F. 2004; Palis, J. & Yoder, M. C. 2001). However, hidden within the uterine tissues, the key steps of early post-implantation development in human largely remain beyond reach, owing to limited access to this stage for both technical and ethical reasons (Pera, M. F. 2017). In vitro models of human embryogenesis have emerged as instrumental platforms to probe human-specific developmental mechanisms (Beccari, L. et al. 2018; Harrison, S. E., et al. 2017, Warmflash, A. et al. 2014; Zheng, Y. et al. 2019; Yu, L. et al. 2021; Sozen, B. et al. 2019; Rivron, N. C. et al. 2018; Liu, X. et al. 2021; Simunovic, M. et al. 2019; Li, R. et al. 2019; Veenvliet, J. V. et al. 2020; Karzbrun, E. et al. 2021; Kagawa, H. et al. 2021). However, they often suffer limitations, such as low efficiency and throughput, limited scalability, and high technical complexity. Traditionally these studies use cocktails of growth factors at supra-physiological levels and encounter challenges to find common media that support diverse cellular fates. Additionally, human post-implantation embryo models with co-developing embryonic and extra-embryonic layers are still missing, which limits the ability to study integrated morphogenetic events of human embryo after implantation. What is needed are in vitro and ex vivo cell culture systems for the study of embryogenesis. The systems and methods disclosed herein address these and other needs.

### SUMMARY

Provided herein are in vitro or ex vivo cell culture systems and uses thereof for generating cellular mass. The cellular mass generated can comprise one or more embryonic cell structures, one or more extraembryonic cell structures, and/or one or more hematopoietic stem cells.

Accordingly, in some aspects, disclosed herein is a method of ex vivo or in vitro generating a cellular mass that comprises one or more embryonic cell structures and one or more extraembryonic cell structures, comprising
a) obtaining a first group of stem cells comprising an inducible transgene encoding a GATA binding protein 6 (GATA6) polypeptide;
b) contacting the first group of stem cells with an inducing agent that induces an expression of the transgene;
c) mixing the first group of stem cells of step b) with a second group of stem cells; and
d) culturing the mixture of cells of step c) on a surface for at least 7 days, thereby producing the cellular mass.

In some embodiments, the second group of stem cells do not have the inducible transgene encoding the GATA6 polypeptide. In some embodiments, the second group of stem cells comprise an inducible transgene encoding an ETS variant transcription factor 2 (ETV2) polypeptide. In some embodiments, step c) further comprises contacting the first group of stem cells of step b) with the second group of stem cells and a third group of cells, wherein the third group of cells comprise an inducible transgene encoding an ETS variant transcription factor 2 (ETV2) polypeptide.

In some embodiments, the first group of stem cells comprise one or more nucleic acid sequences at least about 80% identical to a sequence selected from the group consisting of SEQ ID NOs: 3-10. In some embodiments, the first group of stem cells comprise one or more nucleic acid sequences at least about 80% identical to the sequences of SEQ ID NO: 1 or SEQ ID NO: 2. In some embodiments, the second group of stem cells comprise one or more nucleic acid sequences at least about 80% identical to a sequence selected from the group consisting of SEQ ID NO: 1 and SEQ ID NO: 14. In some embodiments, the second group and/or the third group of stem cells further comprise a nucleic acid sequence at least about 80% identical to SEQ ID NO: 15.

The cellular mass generated by the methods disclosed herein can be used for evaluating, testing, and/or screening agents (e.g., therapeutic agents) that modulate embryogenesis or hematopoietic differentiation, comprising generating a cellular mass that comprises one or more embryonic cell structures and one or more extraembryonic cell structures by the method of any preceding aspects; and contacting the cellular mass with the agent. In some embodiments, the method further comprises determining the biomarkers related to embryogenesis or hematopoietic differentiation.

The embryonic cell structure of the cellular mass can include, but is not limited to, an increased level of an octamer-binding transcription factor 4 (OCT4) polypeptide and a decreased level of a GATA4 polypeptide. The extraembryonic cell structure can include, but is not limited to, an increased level of the GATA6 polypeptide.

In some embodiments, the cellular mass of any preceding aspects comprises one or more of a bilaminar disc-like structure, an amnion-like domain, a primitive streak-like domain, and a yolk sac domain. In some embodiments, the amnion-like domain comprises an increased level of one or more of bone morphogenetic protein 4 (BMP4), bone morphogenetic protein receptor type 1A (BMPR1A), distal-less homeobox 5 (DLX5), follistatin like 1 (FSTL1), inhibitor of DNA binding 1 (ID1), lymphoid enhancer binding factor 1 (LEF1), msh homeobox (MSX)1, MSX2, SMAD family member 1 (SMAD1), and SMAD specific e3 ubiquitin protein ligase 2 (SMURF2).

In some embodiments, the cell mass comprises one or more neural features (including, for example, increased levels of one or more of a cerberus 1 (CER1) polypeptide and left-right determination factor 1 (LEFTY1) polypeptide).

In some embodiments, the hematopoietic stem cells isolated from the cellular mass of any preceding aspects have an increased level of one or more of CD45, CD11b, and CD34.

Also disclosed herein is an in vitro or ex vivo culture system for generating a cellular mass that comprises one or more embryonic cell structures and one or more extraembryonic cell structures, said system comprising
a first group of stem cells that comprise an inducible transgene encoding a GATA binding protein 6 (GATA6) polypeptide; and
a second group of stem cells.

Also disclosed herein is an in vitro or ex vivo culture system for generating a cellular mass that comprises one or more hematopoietic stem cells, said system comprising
a first group of stem cells that comprise an inducible transgene encoding a GATA binding protein 6 (GATA6) polypeptide; and
a second group of stem cells.

In some embodiments, the second group of stem cells do not have the inducible transgene encoding the GATA6 polypeptide. In some embodiments, the second group of stem cells comprise an inducible transgene encoding an ETS variant transcription factor 2 (ETV2) polypeptide. In some embodiments, step c) further comprises contacting the first group of stem cells of step b) with the second group of stem cells and a third group of cells, wherein the third group of cells comprise an inducible transgene encoding an ETS variant transcription factor 2 (ETV2) polypeptide.

Also disclosed herein a method of generating an extracellular matrix, comprising
a) obtaining one or more of a first group of stem cells comprising an inducible transgene encoding a GATA binding protein 6 (GATA6) polypeptide;
b) contacting the one or more first group of stem cells with an inducing agent that induces an expression of the transgene encoding a GATA6 polypeptide;
c) mixing the one or more first group of stem cells of step b) with a second group of stem cells;
d) culturing the mixture of cells of step c) on a surface for at least 3 days, thereby producing a cellular mass; and
e) isolating the extracellular matrix from the cellular mass of step d).

In some embodiments, the second group of stem cells do not have the inducible transgene encoding the GATA6 polypeptide. In some embodiments, the second group of stem cells comprise an inducible transgene encoding an ETS variant transcription factor 2 (ETV2) polypeptide. In some embodiments, step c) further comprises contacting the first group of stem cells of step b) with the second group of stem cells and a third group of cells, wherein the third group of cells comprise an inducible transgene encoding an ETS variant transcription factor 2 (ETV2) polypeptide. In some embodiments, the method of any preceding aspect further comprises contacting the one or more first group of stem cells, the second group of stem cells, and/or the third group of stem cells with an agent (e.g., an agent that improves the signature of trophoblast cells differentiated from the embryonic compartment of the system). In some embodiments, the agent is SB431542.

Also disclosed herein is an in vitro culture system comprising the extracellular matrix generated by the method of any preceding aspects.

### DESCRIPTION OF DRAWINGS

FIGS. 1A-1F show symmetry breaking and fate acquisition following development of iDiscoids (FIG. 1A) Schematic demonstrating cell mixing and subsequent organization of iGATA6 (green) and WT (red) hiPSCs between day 0 and day 5 after GATA6 induction. Arrows on diagrams indicate cell sorting. Scale bar=100 μm (FIG. 1B) Schematic showing sorting of GATA6+ cells from NANOG+ cells from the early (top) to late (bottom) blastocyst stage of the human embryo. (FIG. 1C) Full 8 mm coverslip image of iGATA6/WT co-culture. Scale bar=μm. (FIG. 1D) tSNE projection of clusters with annotations describing the average GATA6 expression level within each cluster. Cluster 5 was further subclustered based on marker expression within a subset of cells of the initial population. (FIG. 1E) Violin plots showing a curated set of genes in iDiscoid clusters. (FIG. 1F) Hypergeometric statistical comparison of differentially expressed gene (DEG) lists between iDiscoid clusters and human E16-19 gastrula-stage populations. Red boxes highlight high DEG similarity to relevant populations of interest.

FIGS. 2A-2E show that iDiscoid generates both primitive and definitive hematopoietic lineages from distinct progenitor populations (FIG. 2A) ELISA comparing secreted AFP and APOA1 detected on D0 and D5 after GATA6 induction with Dox. Individual dots represent biological replicates. (FIG. 2B) Immunofluorescence image showing the distribution of cells expressing CD34 and TAL1 (scl) in iDiscoid culture. Cells expressing TAL1 localize between the yolk sac endoderm compartment and the tissue culture dish and form arrangements of spindle-like cells. Orthogonal slice from shows the position of spindle cells against the dish. Dashed line indicates the position from 1 h the slice was taken. (FIG. 2C) Immunofluorescence image showing cells expressing RUNX1 and TAL1 positioned against the dish. (FIG. 2D) FACS performed on day 5 iDiscoid cultures. CD34−iGATA6 cells generate fewer CD71+ primitive erythroblast-like cells than the CD34+ population, suggesting a greater efficiency of primitive erythropoiesis when cells do not pass through an endothelial state. (FIG. 2E) Schematic depicting in vivo embryo and in vitro iDiscoid yolk sac endoderm and mesoderm layers. Scale bars =100 μm.

FIGS. 3A-3I show that interaction between iGATA6 iPSCs and WT iPSCs leads to amniotic cavity-like lumen formation and expansion (FIG. 3A) Schematic demonstrating progression of lumen formation within the WT clusters. (FIG. 3B) Horizontal and lateral slices showing early rosette formation within a WT cluster. (FIG. 3C) Immunofluorescence showing horizontal and lateral slices of a representative WT cluster. Dashed line indicates the position of z-slice for the image. Solid white arrow indicates the development of a small initial lumen near an area of iGATA6 coverage. Hollow white arrow indicates outward polarization of cells and lack of lumen formation in the area over which there is no iGATA6 coverage. (FIG. 3D) Quantification of characteristics of WT clusters possessing different iGATA6 coverage characteristics. See materials and methods for definitions of coverage types. (FIG. 3E) An orthogonal slice of an individual iDiscoid showing top-bottom compartmentalization of ISL1+ and NANOG+ cells. (FIG. 3F) Schematic showing the position of each population within a single iDiscoid. (FIG. 3G) Horizontal z-slices of the WT cluster from (FIG. 3E). (FIG. 3H) Expression patterns of BMP4 effectors (phosphorylated SMAD1, SMAD5, and SMAD8, left) and a NODAL effector (phosphorylated SMAD2, right) in iDiscoid. (FIG. 3I) Scatterplot showing ISL1 and NANOG expression intensities under control, NODAL inhibition, and BMP4 inhibition conditions. n[control]=87, n[SB431]=122, n[Dorso]=64 Scale bars=100 Mm. n represents separate biological samples from a single experiment harvested on the corresponding day.

FIGS. 4A-4I show that domains expressing the primitive streak markers TBXT and MIXL1 emerge within iDiscoids (FIG. 4A) Immunofluorescence staining showing a TBXT/MIXL1 co-positive domain within the WT clusters of the iDiscoid. (FIG. 4B) Examples of WT clusters with polarized TBXT/MIXL1 domains (top) or without co-expression of markers (bottom). (FIG. 4C) A colored binary image of a day 4 iDiscoid culture showing the positions and types of WT clusters with each characteristic within a single ˜12 mm diameter area. Gray indicates area at the edge of coverslip. Histogram indicates proportions of each domain type from WT clusters from day 4 and day 5. n[D4]=829, n[D5]=184. (FIG. 4D) Distribution of WT cluster areas at day 4 and day 5. Horizontal lines indicate average area per day. n[D4]=263, n[D5]=179 (FIG. 4E) Immunofluorescence showing the position of CER1-expressing iGATA6 cells adjacent to TBXTexpressing WT cells. (FIG. 4F) Schematic showing the location of populations within a single iDiscoid. (FIG. 4G) Scatterplot showing iGATA6 CER1 and WT TBXT expression characteristics on day 4 of iDiscoid culture. Each dot represents one WT cluster. n=(FIG. 4H) Scatterplot showing average intensities of CER1 and GATA6 in the radial area around WT clusters positive for or negative for TBXT domains. n=442 (FIG. 4I) Scatterplot showing CER1 expression versus horizontal cluster area for day 4 (left) and day 5 (right) clusters with and without TBXT expression. Dotted lines indicate horizontal areas of the indicated embryonic stage human embryo. n[D4]=263, n[D5]=180 Scale bars=100 Mm. n represents separate biological samples from a single experiment harvested on the corresponding day.

FIGS. 5A-5C show sorting and symmetry breaking events following GATA6 induction. (FIG. 5A) The gene circuit used to create inducible GATA6-expressing iPSCs (FIG. 5B) Heterogeneity of EGFP (GATA6) activation in iGATA6 cells, detected via FACS. Higher gene circuit copy numbers lead to higher expression level of EGFP and GATA6. (FIG. 5C) Time lapse images of a single position within the iGATA6/wild-type co-culture from day 0 to day 3 after GATA6 induction. Top cropped images correspond to positions within the white boxes in the images below. Scale bar=200 μm.

FIGS. 6A-6C show cell fate divergence across iDiscoid cell types. (FIG. 6A) Immunofluorescence for primitive endoderm markers in day 5 iDiscoid. (FIG. 6B) PDGFRα expression is markedly lower among cells expressing a high level of GATA6 near WT disc. Arrow indicates a cluster of GATA6-high cells on the disc edge that lack PDGFRα. (FIG. 6C) Radial distribution of intensities near the edges of the WT disc. The measurements are taken from roughly two cell diameters within the cluster to four cell diameters outside the cluster (35 μm within, 50 μm outside) The shaded gray area is within the disc; the dotted vertical line marks the distance of the GATA6 intensity peak. Scale bars=100 μm.

FIGS. 7A-7E show subclusters and comparisons to select in vivo populations. (FIG. 7A) Heatmap displaying the top 20 differentially upregulated genes from each of the iDiscoid day scRNAseq clusters. (FIG. 7B) Hypergeometric comparison of differentially expressed genes between iDiscoid day 5 scRNAseq clusters and selected E6.5-8.25 mouse embryo populations. (FIG. 7C) HHEX+ cells are concentrated at the edges of the WT disc, in the area of high GATA6 expression (EGFP). Scale bars=100 μm. (FIG. 7D) tSNE showing isolated cluster 5 obtained as a part of the FindClusters function in Seurat with hierarchical subclustering applied. (FIG. 7E) Selected yolk sac mesoderm and murine allantois-related markers that show expression within a specific subpopulation of cluster 4.

FIGS. 8A-8D show transcriptomic signatures and comparisons to select in vivo populations. (FIG. 8A) Dot plot detailing the expression of yolk sac mesoderm-related ECM and erythrocyte-related proteins in the day 5 iDiscoid system. Highest expression of a most transcripts are concentrated in clusters 5.1 and 5.2. (FIG. 8B) UMAPs showing alignment between human E16-19 embryo populations and the iDiscoid day 5 scRNAseq clusters. Human data taken from Tyser, R. C. V. et al. 2020. (FIG. 8C) UMAP showing label projection of cell identities from reference 17 onto iDiscoid cells. Gray color indicates cells from Tyser, R. C. V. et al. 2020. Identities corresponding to each color can be found from the legend in panel B. (FIG. 8D) Proportion of projected cell types from Tyser, R. C. V. et al. 2020 within each iDiscoid cluster.

FIGS. 9A-9C show identification of endothelial/hematopoietic populations. (FIG. 9A) Immunofluorescence image showing cells expressing hematopoietic markers in iDiscoid culture. Cells expressing the hematopoietic marker TAL1 (Scl) localize between the yolk sac endoderm compartment and the tissue culture. Scale bar=100 μm (FIG. 9B) FACS plots showing the presence of hematopoietic markers on day 14 after a CFU assay for both whole iDiscoid tissue and iDiscoid tissue enriched for CD34-expressing cells. (FIG. 9C) Fraction of live cells expressing the indicated marker or marker combination generated by day 5 iDiscoid following a 14-day CFU assay. Error bars represent SEM, n=3.

FIGS. 10A-10F show iGATA6 migration and laminin deposition over WT clusters. (FIG. 10A) Images showing the day 5 iGATA6 system on day 3 and on day 5 following CXCR4 inhibition (AMD3465) and without (control). Widefield image of a flattened island; iGATA6 cells cover the top of the cluster in the control condition. (FIG. 10B) Representative plot of EGFP expression profiles across the WT cluster on day 3, and on day 5 under control and CXCR4 inhibition (AMD3465) conditions. Shaded areas denote the WT clusters. (FIG. 10C) Violin plots showing the expression of CXCR4 and CXCL12 in scRNAseq clusters. (FIG. 10D) Immunofluorescence showing deposition of laminin at selected days of iDiscoid culture. (FIG. 10E) Immunofluorescence image showing the deposition of laminin around a WT cluster with a central lumen. (FIG. 10F) A three-dimensional reconstruction of a WT cluster with full coverage by iGATA6 cells and inward polarization of WT cells towards a central lumen. The laminin basement membrane can be seen in red on the border between the iGATA6 and WT cells. Scale Bars=100 μm.

FIGS. 11A and 11B show characteristics of epiblast-like clusters during different stages of lumenogenesis. (FIG. 11A) Apoptosis around developing lumens within intermediate-stage clusters. The white dashed outlines show small cavity borders. The green dashed outlines in the second column indicate the area of iGATA6 coverage. Diagrams underneath each column show a lateral view of the lumen formation stage; arrows indicate cell polarity and point towards apical surface, and red stars indicate apoptotic cells. Yellow lines indicate laminin membranes. (FIG. 11B) Diagram demonstrating different stages of WT cluster development. Red cells represent WT; green cells represent iGATA6. Top images within each panel are orthogonal slice view of a Z-stack, with the image from “3. Early” repeated from FIG. 3C. Middle images are diagrams of an orthogonal slice of the system, with yellow representing laminin and blue representing PODXL. The bottom images model the system state in 3D. Yellow line through “late” horizontal image shows the region from which the slice was created. Scale bars=100 μm.

FIGS. 12A and 12B show additional amnion-related expression within epiblast-like cell clusters. (FIG. 12A) Immunofluorescence staining for the amnion markers ISL1 and AP-2a. Top-down widefield image of a flattened coverslip. (FIG. 12B) Horizontal slice through the middle of an iDiscoid bilaminar disc stained with Toluidine blue. WT cells in the inner ring show a columnar morphology. Dotted line shows area of inset. Scale Bars=100 μm.

FIGS. 13A-13C show major signaling interactions between day 5 iDiscoid cell clusters. (FIG. 13A) Dot plot of marker genes from pathways of interest from day (D) 5 iDiscoid scRNAseq. (FIG. 13B) ELISA for BMP4 at D0 and D5 of iDiscoid culture shows an increase in BMP4 secretion. Individual dots represent biological replicates (n=3). Error bars denote SEM. (FIG. 13C) Expression patterns of phosphorylated SMADs following inhibition of NODAL (SB431542, left) and BMP4 (Dorsomorphin, right). ISL1 and NANOG expression in a representative iDiscoid under small molecule inhibitions are shown at the bottom. Lower vertical slices show corresponding markers as well as Hoechst. ISL1 and NANOG expression are lost following NODAL inhibition, but cavity morphology is maintained. ISL1 expression is decreased under BMP4 inhibition, and NANOG expression is maintained. Scale Bars=100 μm.

FIGS. 14A and 14B show signaling leading to Primitive Streak-like Domain Induction. (FIG. 14A) Original image analyzed to create FIG. 5C. Widefield image taken of a coverslip. Scale bar=1000 μm (FIG. 14B) BMP4 signal secreted by the wild-type cells is received by cells at the edges of the disc. In areas in which CER1 inhibits NODAL signaling (from the iGATA6 cells), BMP4 activates mesodermal programs leading to streak-like specification.

FIGS. 15A and 15b show iDiscoid formation from hiPSCs to model human early post-implantation development in vitro. (FIG. 15A) From an initially mixed state, iDiscoid cells segregate into WT clusters surrounded by iGATA6 cells. These iGATA6 cells then sort based on GATA6 expression level, with cells expressing higher GATA6 levels sorted closer to the WT clusters. iGATA6 cells migrate laterally to create a bilaminar boundary on top of the WT clusters. These clusters then undergo lumenogenesis, specification of amnion-like cells, and formation of polarized primitive streak-like domains. (FIG. 15B) Schematic showing the position of each population within a single iDiscoid and their similarity to its in vivo counterpart. iDiscoid is similar to an “unrolled” yolk sac cavity with epiblast that have been separated from their neighboring tissues (i.e., trophoblast and endometrial tissue) and attached to a culture dish.

FIGS. 16A-16C show that mixed iDiscoid cell lines exhibit consistent cluster formation after repeated passaging and cryostorage prior to induction. (FIG. 16A) Schematic showing the creation of the iDiscoid parental cell line. iGATA6 cells with heterogeneous copy numbers of the inducible GATA6 circuit are mixed with wild-type cells at 4:1. This cell combination is then maintained together or frozen prior to induction for iDiscoid experiments. (FIG. 16B) iDiscoid morphology and characteristics following cryostorage and defrosting. (FIG. 16C) Characteristics and morphology of iDiscoid cultures induced immediately after mixing (passage 1) or maintained together and passaged for two months (passage 15). Scale Bars=500 μm.

FIGS. 17A-17E show that iDiscoid engineering in a separate iPSC cell line results in the generation of all major features of interest Day 4 immunofluorescence images of iDiscoid engineered using the iPSC cell line PGP9. (FIG. 17A) PGP9 iGATA6 cells expressing the highest levels of GATA6 near the WT have corresponding low levels of GATA4 (solid arrows), and cells sorted further away with low GATA6 levels have high GATA4 levels (hollow arrows). (FIG. 17B) PGP9 iGATA6 cells expressing high levels of GATA6 (EGFP) also express the anterior endoderm marker HHEX near the edge of a WT cluster. (FIG. 17C) A ring of PODXL expression lines the inside of a cavities formed in PGP9 WT clusters. (FIG. 17D) ISL1 cells specify away from NANOG+ cells, along the base of a cavity formed in PGP9 WT. (FIG. 17E) TBXT+ cells develop polar domains at the edge of WT clusters, near CER1+, GATA6+ areas. Scale Bars=100 μm.

FIG. 18 shows example of FACS gating strategy. (Cited in Methods) Cell debris was excluded via an SSC-A vs FSC-A gate; aggregates were excluded by comparing FSC-A and FSC-H; dead cells were gated out using the 7-AAD stain to identify positive cells.

FIG. 19 shows the map of the construct.

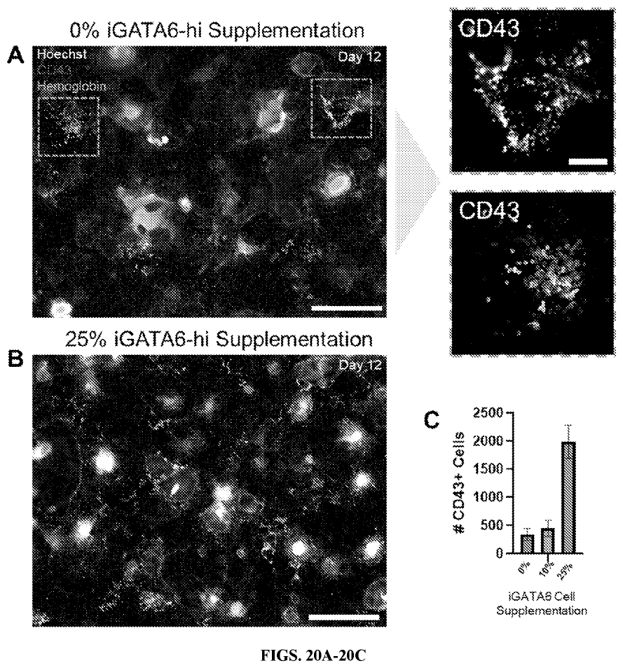

FIGS. 20A-20C show comparison of iDiscoids with and without supplementation with high iGATA6 expressing (iGATA6-hi) cells. FIG. 20A shows expression of CD43⁺ and Hemoglobin⁺ cells in day 12 iDiscoid without additional supplementation of GATA6-hi cells. Red color in high Hoechst areas is nonspecific staining. Scale bar=500 μm. Insets show areas of CD43⁺ cells. Dotted outlines on right show zoomed in areas shown within the image. Scale bar=μm. FIG. 20B shows expression of CD43⁺ and Hemoglobin⁺ cells in day 12 iDiscoid with supplementation of 25% GATA6-hi cells at initial seeding. A significant expansion of CD43-expressing cells is observable. Scale bar=500 μm. A representative image of 2-4 biological replicates. FIG. 20C shows histogram showing the number of CD43⁺ cells detected in experiments in which the given percentages of GATA6-hi cells were substituted. n[0%]=4 replicates, n[10%]=2 replicates, n[25%]=2 replicates.

FIG. 21 shows hematopoietic development with ETV2 supplementation. The development of hemoglobin-expressing cells around ETV2-engineered cells that have taken on an endothelial state subsequent to being supplemented into the system, demonstrating an increase in hematopoietic and endothelial activity in the presence of this cell type.

FIGS. 22A-22C show hematopoietic development with ETV2 supplementation. FIG. 22A shows development of spherical hematopoietic-like cells within CD34-expressing endothelial cells, mimicking blood island development. FIG. 22B shows Z-slices of a day 12 iDiscoid at the indicated height above the bottom focal plane. Expression of the mesodermal marker Desmin is regionalized close to the bottom of the tissue; endothelial marker CD34 is expressed near the bottom and middle of the tissue, with highest expression just above Desmin (z=6-8); endoderm marker FOXA2 is exclusively expressed above the other tissues. Scale bar=50 μm. FIG. 22C shows Z-slices around the blood island-like area shown in FIG. 4E. Desmin is observed regionalized below the endothelial (CD31⁺) and hematopoietic (CD43⁺) tissues. No stain for endoderm is shown. Scale bar=100 μm.

FIGS. 23A-23C show examples of TBXT and CER1 anterior and posterior cells observed in iDiscoids. FIG. 23A shows representative WT cluster showing syn-polarity of TBXT and CER1 within the WT cluster and iGATA6 layers, respectively. Filled arrow indicates CER1-expressing cells within the iGATA6 layer; empty arrow indicates CER1 and TBXT co-expressing cells within the WT layer. Z-slices are representative slices from the center of two different WT discs. FIG. 23B shows representative WT clusters showing anti-polarity of TBXT and CER1 within the WT cluster and iGATA6 layers, respectively. Filled arrow indicates CER1-expressing cells within the iGATA6 layer; empty arrow indicates CER1 and TBXT co-expressing cells within the WT layer. Z-slice is a representative slice from the center of a WT disc. FIG. 23C shows merged UMAPs of all D0-D5 scRNA-seq data showing the posterior compartment expressing the inhibitor CER1. Dotted boxes show WT lineages. Scale Bars=100 μm.

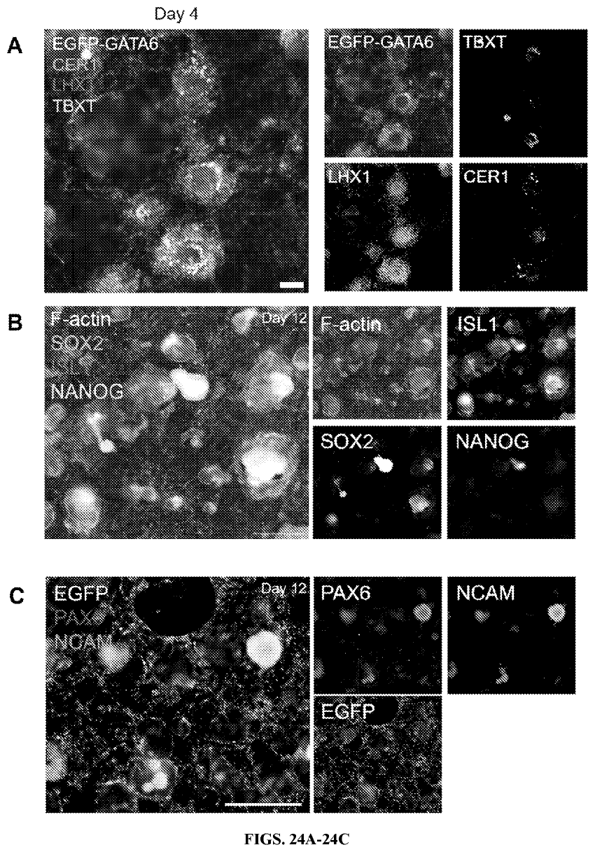

FIGS. 24A-24C show effects of BMP4 inhibition on anterior-like and posterior domains in iDiscoids. FIG. 24A shows control iDiscoids showing development of TBXT⁺ posterior domains and LHX1⁺ areas expressing CER1. FIG. 24B shows most structures corresponding to former WT clusters at day 12 in iDiscoid have taken on expression of ISL1 and lost expression of the pluripotency markers SOX2 and NANOG. A limited number of cells express SOX2 at the core of unengineered clusters, potentially indicating the specification of an ectoderm-like fate in a small number of WT-lineage cells. FIG. 24C shows a subset of unengineered clusters at day 12 have taken on markers of surface ectoderm and neural crest differentiation. Scale bars=100 μm.

FIGS. 25A-25H show identification of endothelial/hematopoietic populations at day 5 and day 12 within the system. FIG. 25A shows scatterplots showing the distribution of markers obtained via image analysis of 3 independent experiments at day 5. Percentages correspond to the fraction of cells recorded with corresponding marker expression levels above the thresholds represented by the dotted lines. FIG. 25B shows immunofluorescence image showing cells expressing hematopoietic markers in iDiscoid culture. Cells expressing the hematopoietic marker TAL1 (Scl) localize between the yolk sac endoderm compartment and the tissue culture dish. Scale bar=100 μm. FIG. 25C shows immunofluorescence image showing cells expressing RUNX1 and TAL1 positioned against the dish. Scale bar=100 μm. FIG. 25D shows image analysis of z-slices from 5 areas in 3 biological replicates. The peak of ERG expression is underneath the peak of EGFP expression representing the iGATA6 endoderm layer. Dotted curves represent SEM calculated at each point. FIG. 25E shows immunofluorescence image showing cells expressing VE-cadherin and VEGFR2 positioned against the dish. Arrow indicates likely differentiating endothelial cell from the overlaying iGATA6 layer. Scale bar=100 μm. FIG. 25F shows flow cytometry plots showing the presence of hematopoietic markers on day 14 after a CFU assay initiated CD34-expressing cells (enriched from iDiscoids). FIG. 25G shows results from image analysis demonstrating the change in area of CD34⁺ cells between day 5 and day 12 of iDiscoid culture, assessed via analysis of immunofluorescence images. Individual dots represent biological replicates. **: P value=0.0055. FIG. 25H shows representative flow cytometry plot on day 5 and day 12 of iDiscoid showing expansion of the CD34⁺ population by day 12 (n=3).

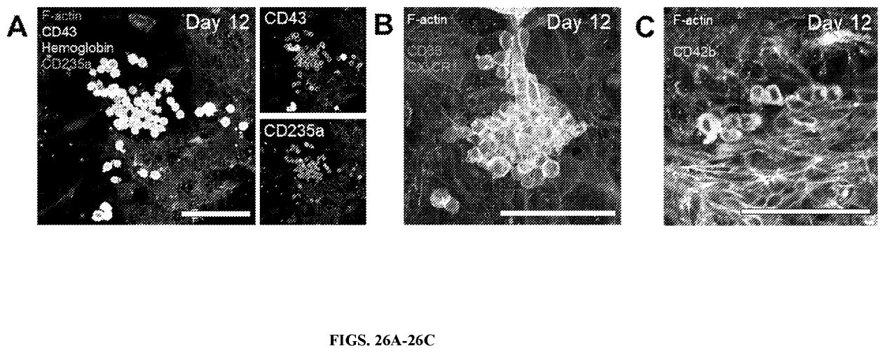

FIGS. 26A-26G show hematopoietic cells develop over long term culture within the system. FIG. 26A shows erythroid markers (CD235a and Hemoglobin) expressed in a subset of CD43⁺ round cells in day 12 iDiscoids. Scale bar=100 μm. FIG. 26B shows cells expressing myeloid (CD31, CD33) and macrophage (CX₃CR1) markers in a representative cluster in day 12 iDiscoid. Scale bar=100 μm. FIG. 26C shows cells expressing megakaryocyte markers in day iDiscoid. Scale bar=100 μm. FIG. 26D shows UMAP showing unbiased clustering of scRNAseq performed on day 21. Boxed area shows populations expressing hematopoietic marker genes. FIG. 26E shows hypergeometric statistical comparison of day 21 iDiscoid populations to the E16-19 human embryo populations from Tyser et al. 2022. The red box shows populations of interest with similarity to in vivo hematopoietic lineages. iDiscoid clusters are grouped by similar fates on Y axis. FIG. 26F shows expression of hematopoietic marker genes within the red boxed population in FIG. 26D. The four clusters show distinct marker gene expression profiles associated with yolk sac-derived hematopoietic populations. FIG. 26G shows flow cytometry analysis on day 21 iDiscoid. Cells were gated for positive CD43 expression prior to gating for the markers shown (n=2, representative sample shown).

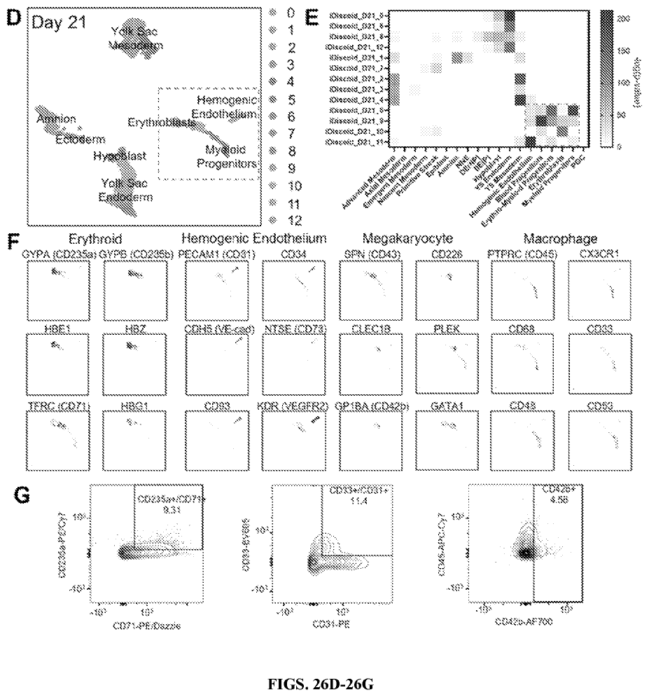

FIGS. 27A-27D show major signaling interactions between day 5 iDiscoid cell clusters. FIG. 27A shows immunofluorescence staining for the amnion markers ISL1 and ^{A}P-2α at day 4. Top-down widefield image of a flattened coverslip. Scale Bar=100 μm. FIG. 27B shows Dot plot of marker genes from the BMP pathway from day 4 iDiscoid scRNA-seq. BMP4 expression and a number of associated genes (boxed in red) are highest in the amnion-like population. FIG. 27C shows merged UMAP of all WT lineages from iDiscoid labeled by day of development. FIG. 27D shows merged WT population showing the compartment expressing markers of amnion. ISL1, TFAP2A (AP-2^{α}), and GATA3 are expressed in this area, while NANOG is negative and OCT4 is low.

FIGS. 28A-28B show flow cytometry showing reconstitution of mouse spleen and bone marrow with human hematopoietic cells of multiple lineages. FIG. 28A shows flow cytometry on mouse bone marrow following retro-orbital injection of day 5 iGATA6 system tissue showing the reconstitution of the murine hematopoietic stem cell population within the bone marrow with up to 0.084% human cells. FIG. 28B shows flow cytometry on mouse spleen following retro-orbital injection of day 5 iGATA6 system tissue showing the reconstitution of the murine hematopoietic stem cell population within the bone marrow with up to 5.76% human cells.

FIGS. 29A-29E show Day 4 scRNAseq after SB431542 application at day 2. FIG. 29A shows UMAP showing the merged iDiscoid clusters with unsupervised clustering applied. Under inhibition with SB431542, the WT cluster divides into two clusters. Clusters are ordered from lowest to highest GATA6 expression. FIG. 29B shows violin plots showing the distribution of markers within the clusters shown. NANOG expression is lost within the WT while the other pluripotency markers SOX2 and OCT4 are retained. Expression of the amnion marker ISL1 is present in cluster 2 alongside CDX2, GATA3, and AP-2γ. FIG. 29C shows hypergeometric statistical comparison to the annotated human embryo populations from Xiang et al. 2020. Cluster shows little similarity to trophoblast fates. FIG. 29D shows hypergeometric statistical comparison to the annotated cynomolgus embryo populations from Ma et al. 2019. Cluster 2 shows high similarity to the early amnion population (E-AM) and lower similarity to late amnion and epiblast identities. FIG. 29E shows hypergeometric statistical comparison to the annotated human embryo populations from Tyser et al. 2022. Cluster 2 shows uniquely high similarity to the amnion identity. iGATA6 clusters 0, 3, and 5 retain strong statistical similarity to yolk sac endoderm, and the GATA6-high cluster 4 acquires a yolk sac mesoderm-like identity. The putative anterior identity does not clearly segregate from the yolk sac endoderm identity as observed in Day 4 iDiscoid without inhibition, nor does the posterior emerge within the WT clusters.

FIGS. 30A-30C show hypergeometric statistical comparison of iDiscoid time points to human and NHP embryo data. FIG. 30A shows hypergeometric statistical comparison of each iDiscoid day to the annotated human embryo populations from Tyser et al. 2022. Blue dots above each column indicates the relative GATA6 expression level of each population indicated per day. Followin induction of the iGATA6 cells, the unengineered cells (left columns) move from a state not similar to the human embryo to a state similar to the human epiblast. FIG. 30B shows hypergeometric statistical comparison of each iDiscoid day to the annotated human embryo populations from Xiang et al. 2020. Scale used is the same as shown in FIG. 30A. FIG. 30C shows hypergeometric statistical comparison of each iDiscoid day to the annotated cynomolgus embryo populations from Ma et al. 2019. Scale used is the same as shown in FIG. 30A. iDiscoid clusters correspond to those in the individual day-by-day clustering and are ordered from left to right on the x-axis by lowest to highest GATA6 expression level. “W” clusters are clusters with putative wild-type lineage; “G” clusters are clusters with putative iGATA6 lineage. Dotted outlines indicate fate comparisons of the most interest for each cluster. Abbreviations from other datasets: NNE=Non-neural ectoderm, DE (P)=Definitive endoderm (proliferative), DE (NP)=Definitive endoderm (not proliferative), YS=Yolk sac, PGC=Primordial germ cell, CTB=Cytotrophoblast, STB=Syncytiotrophoblast, EVT=Extravillous trophoblasts, PSA-EPI=Primitive streak anlage in the epiblast, EXMC=Extraembryonic mesoderm cells, E- =Early, L-=Late, Gast=Gastrulating cells, AM=Amnion, VE/YE=Visceral endoderm/Yolk sac endoderm.

### DETAILED DESCRIPTION

Disclosed herein an in vitro cell culture system and uses thereof for generating a cellular mass, wherein said system comprises one or more of a first group of stem cells that comprise an inducible transgene encoding a GATA binding protein 6 (GATA6) polypeptide and a second group of stem cells. This method has been shown to be surprisingly effective at generating a cellular mass that comprises one or more embryonic cell structures and one or more extraembryonic cell structures from stem cells. In some examples, the generated cellular mass comprises one or more of a bilaminar disc-like structure, an amnion-like domain, a primitive streak-like domain, and a yolk sac domain.

### Terminology

As used herein, the terms “can,” “optionally,” and “can optionally” are used interchangeably and are meant to include cases in which the condition occurs as well as cases in which the condition does not occur. Thus, for example, the statement that a formulation “can include an excipient” is meant to include cases in which the formulation includes an excipient as well as cases in which the formulation does not include an excipient.

As used in the specification and claims, the singular form “a,” “an,” and “the” include plural references unless the context clearly dictates otherwise. For example, the term “a cell” includes a plurality of cells, including mixtures thereof.

The term “about” as used herein when referring to a measurable value such as an amount, a percentage, and the like, is meant to encompass variations of ±20%, ±10%, ±5%, or ±1% from the measurable value.

“Administration” to a subject or “administering” includes any route of introducing or delivering to a subject an agent. Administration can be carried out by any suitable route, including oral, intravenous, intraperitoneal, and the like. Administration includes self-administration and the administration by another.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed.

Contacting: Placement in direct physical association, for example solid, liquid or gaseous forms. Contacting includes, for example, direct physical association of fully- and partially-solvated molecules.

A “control” is an alternative subject or sample used in an experiment for comparison purposes. A control can be “positive” or “negative.”

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom, Thus, a gene encodes a protein if transcription and translation of mRNA.

The term “engineered” and other grammatical forms thereof as used herein may refer to one or more changes of nucleic acids, such as nucleic acids within the genome of an organism. The term “engineered” may refer to a change, addition and/or deletion of a gene. “Engineered cells” can also refer to cells that contain added, deleted, and/or changed genes.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.) The “fragments,” whether attached to other sequences or not, can include insertions, deletions, substitutions, or other selected modifications of particular regions or specific amino acids residues, provided the activity of the fragment is not significantly altered or impaired compared to the nonmodified peptide or protein. These modifications can provide for some additional property, such as to remove or add amino acids capable of disulfide bonding, to increase its bio-longevity, to alter its secretory characteristics, etc. In any case, the fragment must possess a bioactive property, such as regulating the transcription of the target gene.

The term “gene” or “gene sequence” refers to the coding sequence or control sequence, or fragments thereof. A gene may include any combination of coding sequence and control sequence, or fragments thereof. Thus, a “gene” as referred to herein may be all or part of a native gene. A polynucleotide sequence as referred to herein may be used interchangeably with the term “gene”, or may include any coding sequence, non-coding sequence or control sequence, fragments thereof, and combinations thereof. The term “gene” or “gene sequence” includes, for example, control sequences upstream of the coding sequence (for example, the ribosome binding site).

The term “genetically engineered cell” as used herein refers to a cell modified by means of genetic engineering.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) nucleotide sequence identity is defined as the percentage of amino acids in a candidate sequence that are identical to the nucleotides in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01.

The term “increased” or “increase” as used herein generally means an increase by a statically significant amount; for the avoidance of any doubt, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level so long as the increase is statistically significant.

The term “isolating” as used herein refers to isolation from a biological sample, i.e., blood, plasma, tissues, exosomes, or cells. As used herein the term “isolated,” when used in the context of, e.g., a cell, refers to a cell of interest that is at least 60% free, at least 75% free, at least 90% free, at least 95% free, at least 98% free, and even at least 99% free from other compounds, materials, matter, mass and/or substances with which the cell is associated with prior to purification.

By the term “modulating,” as used herein, is meant mediating a detectable increase or decrease in the level of an expression level or response in a cell or subject compared with the level of an expression level or response in the cell or subject in the absence of a treatment or compound, and/or compared with the level of expression or response in an otherwise identical but untreated cell or subject. The term encompasses perturbing and/or affecting a native signal, native expression level, or response thereby mediating a beneficial therapeutic response in a subject, preferably, a human.

The term “neural feature” is used herein to refer to increased expression of markers related to neural plate and surface ectoderm such as the markers cerberus 1 (CER1) and left-right determination factor 1 (LEFTY1).

The term “nucleic acid” as used herein means a polymer composed of nucleotides, e.g. deoxyribonucleotides (DNA) or ribonucleotides (RNA). The terms “ribonucleic acid” and “RNA” as used herein mean a polymer composed of ribonucleotides. The terms “deoxyribonucleic acid” and “DNA” as used herein mean a polymer composed of deoxyribonucleotides.

The term “polynucleotide” refers to a single or double stranded polymer composed of nucleotide monomers.

The term “polypeptide” refers to a compound made up of a single chain of D- or L-amino acids or a mixture of D- and L-amino acids joined by peptide bonds.

The term “promoter” or “regulatory element” refers to a region or sequence determinants located upstream or downstream from the start of transcription and which are involved in recognition and binding of RNA polymerase and other proteins to initiate transcription. Promoters need not be of bacterial origin, for example, promoters derived from viruses or from other organisms can be used in the compositions, systems, or methods described herein.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia, PA, 2005. Examples of physiologically acceptable carriers include saline, glycerol, DMSO, buffers such as phosphate buffers, citrate buffer, and buffers with other organic acids; antioxidants including ascorbic acid; low molecular weight (less than about residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, New Jersey), polyethylene glycol (PEG), and PLURONICS™ (BASF; Florham Park, NJ). To provide for the administration of such dosages for the desired therapeutic treatment, compositions disclosed herein can advantageously comprise between about 0.1% and 99% by weight of the total of one or more of the subject compounds based on the weight of the total composition including carrier or diluent.

The term “tissue” refers to a group or layer of similarly specialized cells which together perform certain special functions. The term “tissue” is intended to include, blood, blood preparations such as plasma and serum, bones, joints, muscles, smooth muscles, lung tissues, and organs.

The term “transgene” refers to a gene that is artificially introduced into another organism or cell. An “inducible transgene” is a transgene that is only expressed in a desired amount in the presence of an inducing agent. In one embodiment, the inducing agent is doxycycline.

“Transplant” refers to a biocompatible lattice or a donor tissue, organ or cell, to be transplanted. An example of a transplant may include but is not limited to skin cells or tissue, bone marrow, and solid organs such as heart, pancreas, kidney, lung and liver. A transplant can also refer to any material that is to be administered to a host. For example, a transplant can refer to a nucleic acid or a protein.

The terms “treat,” “treating,” “treatment,” and grammatical variations thereof as used herein, include partially or completely delaying, alleviating, mitigating or reducing the intensity of one or more attendant symptoms of a disorder or condition and/or alleviating, mitigating or impeding one or more causes of a disorder or condition. Treatments according to the invention may be applied preventively, prophylactically, pallatively or remedially. Prophylactic treatments are administered to a subject prior to onset, during early onset, after an established development of a disease or a disorder. Prophylactic administration can occur for several minutes to months prior to the manifestation of a disease or a disorder. In some instances, the terms “treat,” “treating,” “treatment,” and grammatical variations thereof, include mitigating a disease, and/or related symptoms in a subject as compared with prior to treatment of the subject or as compared with incidence of such symptom in a general or study population.

### Cell Culture System

Disclosed herein is an in vitro or ex vivo culture system for generating a cellular mass that comprises one or more embryonic cell structures and one or more extraembryonic cell structures or one or more hematopoietic stem cells, said system comprising:
one or more of a first group of stem cells that comprise an inducible transgene encoding a GATA binding protein 6 (GATA6) polypeptide; and
a second group of stem cells.

The embryonic cell structure is comprised of the pluripotent stem cells and their derivatives, including, for example, epiblast-like cells (marked by one or more of OCT4, NANOG, SOX2, and DPPA4), an amnion-like domain (marked by one or more of ISL1, BMP4, DLX5, ID1, BMPR1A, FSTL1, LEF1, and MSX1), and/or a primitive streak-like domain (marked by oen or more of TBXT and MIXL1). The extraembryonic cell structure is comprised of, including, for example, yolk sac endoderm (marked by one or more of GATA6, GATA4, PDGFRA, SOX17, and FOXA2), yolk sac mesoderm (marked by one or more of BST2, COL2A1, VIM, and DES), anterior visceral endoderm (marked by one or more of CER1, HHEX, LHX1, and GSC), yolk sac endothelium (marked by one or more of CD34, PECAM1, CDH5, and ETV2), yolk sac hemogenic endothelium (marked by one or more of CD34, RUNX1, TAL1, ERG, and ETV2), hematopoietic cells (marked by one or more of CD34, CD43, CD45, RUNX1, CD71, CD33, CX3CR1, and CD41).

The first group of stem cells comprising an inducible transgene encoding a GATA6 polypeptide can further comprise additional transcriptional factor that are expressed by the stem cells other than GATA6, including, for example, GATA4, SOX17, SOX7, TBX5, PDX1, HNF4A, FOXA2, MYOD, LMO2, GATA3, GATA1, GATA2, KLF2, KLF1, SOX9, SOX4, and/or ERG.

It should be understood and herein contemplated that the one or more first groups of stem cells comprising the inducible transgene are generated by an inducible expression system. By “inducible expression system” is meant all of the components required to perform an inducible expression protocol, i.e., a protocol in which expression of a coding sequence of a cell occurs in response to an applied stimulus, e.g., contact with an expression mediator compound (e.g., doxycycline), as compared to a constitutive expression protocol, i.e. a protocol in which expression of a coding sequence (also termed transcriptional unit) in a cell is continuous irrespective of the presence or absence of a particular expression mediator component. Inducible expression systems of the invention are those that include a transcription modulator (e.g., rtTA) and a transcription modulator responsive element (e.g., tetracycline-responsive element or TRE), where the transcription modulator binds to the transcription modulator responsive element (in some instances in the presence of an expression mediator) to controllably induce expression of a coding sequence (i.e., transcriptional unit), as desired.

In some embodiments, the one or more first group of stem cells comprise a doxycycline inducible transgene that encodes a GATA6 polypeptide.

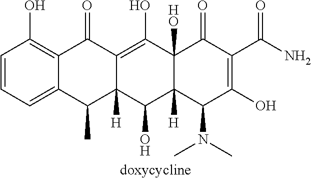 [SMILES: C[C@H]1c2cccc(O)c2C(=O)C2=C(O)[C@]3(O)C(=O)C(C(N)=O)=C(O)[C@@H](N(C)C)[C@@H]3[C@@H](O)[C@@H]21]

In some embodiments, the stem cells disclosed herein comprise any inducible system known in the art, including, for example, GAL4 (Promoter: UAS, which is CGG-N11-CCG, where N can be any base), Synthetic Zinc Finger (Promoter: Synthetic promoter designed to match the zinc finger sequence), TMP (For the activation of DD-Cre for recombination of “floxed” genes to enable expression from a constitutive promoter), Tamoxifen (For the activation of Cre recombinase for recombination of “floxed” genes to enable expression from a constitutive promoter), Cumate (promoter: Cumate Operator [CuO]), 17β-Estradiol or Synthetic Activator (SynX) (Promoter: Estrogen Receptor alpha).

In some embodiments, the one or more first groups of stem cells each comprises the one or more inducible transgenes encoding the one or more GATA6 polypeptides. In some embodiments, the GATA6 polypeptide is encoded by one or more sequences at least about 80% (at least about 80%, 85%, 90%, 95%, 98%, or 99%) identical to a sequence selected from the group of SEQ ID NOs: 3-10 or a fragment thereof. In some embodiments, the transgene comprises a sequence at least about 80% (at least about 80%, 85%, 90%, 95%, 98%, or 99%) identical to SEQ ID NO: 10 or a fragment thereof. In some embodiments, the one or more first groups of stem cells each comprises one or more transcription modulator responsive elements (e.g., tetracycline-responsive element or TRE). In some embodiments, the transcription modulator responsive element comprises one or more sequences at least about 80% (at least about 80%, 85%, 90%, 95%, 98%, or 99%) identical to SEQ ID NO: 11 or a fragment thereof.

In some embodiments, the one or more first group of stem cells, the second group of stem cells, and/or the third group of stem cells are in contact with an agent (e.g., an agent that improves the signature of trophoblast cells differentiated from the embryonic compartment of the system). In some embodiments, the agent is SB431542.

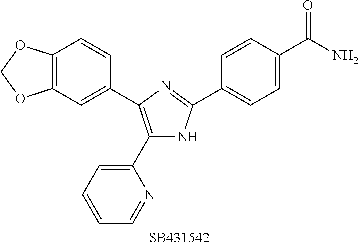 [SMILES: NC(=O)c1ccc(-c2nc(-c3ccc4c(c3)OCO4)c(-c3ccccn3)[nH]2)cc1]

In some embodiments, the one or more first group of stem cells comprise a nucleic acid sequence at least about 80% (at least about 80%, about 85%, about 90%, about 95%, about 98%, or about 99%) identical to SEQ ID NO: 1 or a fragment thereof. In some embodiments, the one or more first group of stem cells comprise a nucleic acid sequence at least about 80% (at least about 80%, about 85%, about 90%, about 95%, about 98%, or about 99%) identical to SEQ ID NO: 2 or a fragment thereof. In some embodiments, the second group of stem cells comprise a nucleic acid sequence at least 80% (at least about 80%, about 85%, about 90%, about 95%, about 98%, or about 99%) identical to SEQ ID NO: 1 or a fragment thereof.

In some embodiments, the second group of stem cells do not have the inducible transgene encoding the GATA6 polypeptide.

In some embodiments, the second group of stem cells further comprise an inducible transgene encoding an ETS variant transcription factor 2 (ETV2) polypeptide. Accordingly, in some aspects, disclosed herein is an in vitro or ex vivo culture system for generating a cellular mass that comprises one or more embryonic cell structures and one or more extraembryonic cell structures or one or more hematopoietic stem cells, said system comprising:
one or more first groups of stem cells that comprise an inducible transgene encoding a GATA binding protein 6 (GATA6) polypeptide; and
a second group of stem cells, wherein the second group of stem cells further comprise an inducible transgene encoding an ETS variant transcription factor 2 (ETV2) polypeptide.

In some embodiments, the in vitro or ex vivo culture system disclosed herein further comprises a third group of cells, wherein the third group of cells comprise an inducible transgene encoding an ETS variant transcription factor 2 (ETV2) polypeptide. Accrordingly, in some aspects, disclosed herein is an in vitro or ex vivo culture system for generating a cellular mass that comprises one or more embryonic cell structures and one or more extraembryonic cell structures or one or more hematopoietic stem cells, said system comprising:
one or more first groups of stem cells that comprise an inducible transgene encoding a GATA binding protein 6 (GATA6) polypeptide;
a second group of stem cells; and
a third group of cells, wherein the third group of cells comprise an inducible transgene encoding an ETS variant transcription factor 2 (ETV2) polypeptide.

Organoids generated from single cell clonally derived iGATA6 or iETV2 engineered lines can show augmented features that are superior and different from those observed in a heterogeneous line, as a result of a different copy number of the gene circuit. As a result, they can be used for a plug-and-play approach for generating defined tissues of interest with specific functional capacity in a way that is distinct from organoids derived from heterogeneous iGATA6 mixtures. Different clonal lines can have a different gene circuit copy number and different ranges of expression (or expression noise) that can be exploited for engineering designer tissues with augmented behavior, such as to improve blood production, improve mesoderm subsets, or improve vascular subsets. Accordingly, in some embodiments, the one or more first groups of stem cells comprise different numbers of the inducible transgenes encoding the GATA6 polypeptides. In some embodiments, the one or more first groups of stem cells comprise the same number of the inducible transgenes encoding the GATA6 polypeptides. It should be understood and herein contemplated that the one or more first groups of stem cells express different levels of the GATA6 polypeptides. The different expression levels of the GATA6 polypeptides can be achieved by delivering the transgene encoding the GATA6 polypeptide one or more times (e.g., one, two, three, four, five, six, seven, eight, nine, or ten times or more) to different first groups of stem cells. The transgene can be delivered by any expression vectors known in the art (e.g., a DNA vector, a viral vector, or a nanoparticle). In some embodiments, the different expression levels of the GATA6 polypeptides can be achieved by delivering the transgene comprising one or more copies of GATA6 polynucleotides (e.g., one, two, three, four, five, six, seven, eight, nine, or ten copies or more) to different first groups of stem cells. In some embodiments, the different expression levels of the GATA6 polypeptides can be achieved by delivering one or more RNA sequences (e.g., one, two, three, four, five, six, seven, eight, nine, or ten RNA sequences or more) encoding the GATA6 polypeptide. In some embodiments, the different expression levels of the GATA6 polypeptides can be achieved by delivering one or more GATA6 polypeptides (e.g., one, two, three, four, five, six, seven, eight, nine, or ten GATA6 polypeptides or more).

In some embodiments, the one or more first groups of cells and second group of cells are pluripotent stem cells. As used herein, the term “pluripotent stem cell” refers to a cell capable of continued self-renewal and capable of, under appropriate conditions, differentiating into cells of all three germ layers. Examples of pluripotent stem cells (PSCs) include embryonic stem cells (ESCs) and induced pluripotent stem cells (iPSCs). As used herein, “embryonic stem cells” or “ESCs” mean a pluripotent cell or population of pluripotent cells derived from an inner cell mass of a blastocyst. See, e.g., Thomson et al, Science 282: 1145-1147 (1998). These cells express Oct-4, S SEA-3, S SEA-4, TRA-1-60, and TRA-1-81. As used herein, the term “induced pluripotent stem cell” or “iPSC” refers to a pluripotent cell or population of pluripotent cells that may vary with respect to their differentiated somatic cell of origin, that may vary with respect to a specific set of potency determining factors, and that may vary with respect to culture conditions used to isolate them, but nonetheless are substantially genetically identical to their respective differentiated somatic cell of origin and display characteristics similar to higher potency cells, such as ESCs, as described herein. See, e.g., Yu et al., Science 318: 1917-1920 (2007). In some embodiments, the one or more first group of cells and second group of cells are induced pluripotent stem cells (iPSCs). In some embodiments, the one or more first group of cells and second group of cells are human cells.

In some embodiments, the first group of stem cells and the second group of stem cells are mixed at ratio of about 1:20 to about 1:2, preferably about 1:10 to about 1:3, or preferably about 1:10 to about 1:4.

The transgenes, the polynucleotides, or the polypeptides (e.g., GATA6 and ETV2) can be delivered to the stem cells or a subject in need through any known route of delivery, including, for example, transfection-based delivery of mRNA of GATA6 or ETV2, or protein delivery of GATA6 or ETV2. DNA plasmid vectors can also be delivered to engineering cells with a stable integrated gene circuit introduced via piggybac transposase. In some examples, the cells can be generated in vitro and then be transplanted in vivo. After transplantation, the cells can be induced in vivo.

### Methods

Also disclosed herein is a method of ex vivo or in vitro generating a cellular mass that comprises one or more embryonic cell structures and one or more extraembryonic cell structures, comprising
a) obtaining one or more of a first group of stem cells comprising an inducible transgene encoding a GATA binding protein 6 (GATA6) polypeptide;
b) contacting the one or more first groups of stem cells with an inducing agent that induces an expression of the transgene;
c) mixing the one or more first groups of stem cells of step b) with a second group of stem cells; and
d) culturing the mixture of cells of step c) on a surface for at least 7 days, thereby producing the cellular mass.

As used herein, the term “cellular mass” refers to a mass of cells with a distinct boundary. The cellular mass can be in a two-dimensional structure or a three-dimensional structure. In some embodiments, the cellular mass comprises one or more embryonic cell structures and one or more extraembryonic cell structures. In some embodiments, the cellular mass comprises one or more hematopoietic stem cells.

In some embodiments, step b) comprises contacting the one or more first group of stem cells with the agent for at least about 2 days, 3 days, 4 days, 5 days, 6 days, 7 days, 8 days, 9 days, 10 days, 11 days, 12 days, 13 days, 14 days, 15 days, 18 days, 20 days, 25 days, or 30 days, or about 2 to 7 days, about 4 to 9 days, about 6 to 12 days, about 8 to 14 days, about 10 to 16 days, about 12 to 18 days, about 14 to 20 days, about 16 to 22 days, about 18 to 24 days, about 20 to 26 days, about 16 to 30 days, about 2 to 4 days, about 3 to 5 days, about 4 to 6 days, about 5 to 7 days, about 6 to 9 days, or about 7 to 10 days.

In some embodiments, the one or more first groups of stem cells each comprises the one or more inducible transgenes encoding the one or more GATA6 polypeptides. In some embodiments, the GATA6 polypeptide is encoded by one or more sequences at least about 80% (at least about 80%, 85%, 90%, 95%, 98%, or 99%) identical to a sequence selected from the group consisting of SEQ ID NOs: 3-10 or a fragment thereof. In some embodiments, the GATA6 transgene comprises a sequence at least about 80% (at least about 80%, 85%, 90%, 95%, 98%, or 99%) identical to SEQ ID NO: 10 or a fragment thereof. In some embodiments, the one or more first groups of stem cells each comprise one or more transcription modulator responsive elements (e.g., tetracycline-responsive element or TRE). In some embodiments, the transcription modulator responsive element comprises one or more sequences at least about 80% (at least about 80%, 85%, 90%, 95%, 98%, or 99%) identical to SEQ ID NO: 11 or a fragment thereof. In some embodiments, the one or more first groups of stem cells comprise different numbers of the inducible transgenes encoding the GATA6 polypeptides. In some embodiments, the one or more first groups of stem cells comprise the same number of the inducible transgenes encoding the GATA6 polypeptides. It should be understood and herein contemplated that in some embodiments, the one or more first groups of stem cells express different levels of the GATA6 polypeptides. In some embodiments, the one or more first groups of stem cells comprise a doxycycline inducible transgene that encodes a GATA6 polypeptide. The different expression levels of the GATA6 polypeptides can be achieved by delivering the transgene encoding the GATA6 polypeptide one or more times to different first groups of stem cells. The transgene can be delivered by any expression vectors known in the art (e.g., a DNA vector, a viral vector, or a nanoparticle). In some embodiments, the different expression levels of the GATA6 polypeptides can be achieved by delivering one or more RNA sequences encoding the GATA6 polypeptide. In some embodiments, the different expression levels of the GATA6 polypeptides can be achieved by delivering one or more GATA6 polypeptides.

The first group of stem cells comprising an inducible transgene encoding a GATA6 polypeptide can further comprise additional transcriptional factor that are expressed by the stem cells other than GATA6, including, for example, GATA4, SOX17, SOX7, TBX5, PDX1, HNF4A, FOXA2, MYOD, LMO2, GATA3, GATA1, GATA2, KLF2, KLF1, SOX9, SOX4, and/or ERG.

In some embodiments, the one or more first group of stem cells comprise a doxycycline inducible transgene that encodes a GATA6 polypeptide. The concentration of doxycycline in step b can be from about 1 ng/ml to about 10,000 ng/ml, Ing/ml to about 1000 ng/ml, from about 10 ng/ml to about 750 ng/ml, from about 100 ng/ml to about 500 ng/ml, from about 100 ng/ml to about 250 ng/ml, or from about 10 ng/ml to about 150 ng/ml.

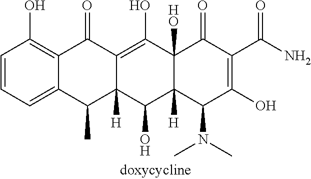 [SMILES: C[C@H]1c2cccc(O)c2C(=O)C2=C(O)[C@]3(O)C(=O)C(C(N)=O)=C(O)[C@@H](N(C)C)[C@@H]3[C@@H](O)[C@@H]21]

In some embodiments, the stem cells disclosed herein comprise any inducible system known in the art, including, for example, GAL4 (Promoter: UAS, which is CGG-N11-CCG, where N can be any base), Synthetic Zinc Finger (Promoter: Synthetic promoter designed to match the zinc finger sequence), TMP (For the activation of DD-Cre for recombination of “floxed” genes to enable expression from a constitutive promoter), Tamoxifen (For the activation of Cre recombinase for recombination of “floxed” genes to enable expression from a constitutive promoter), Cumate (promoter: Cumate Operator [CuO]), 170-Estradiol or Synthetic Activator (SynX) (Promoter: Estrogen Receptor alpha).

In some embodiments, the one or more first groups of stem cells comprise a nucleic acid sequence at least about 80% (at least about 80%, about 85%, about 90%, about 95%, about 98%, or about 99%) identical to SEQ ID NO: 1 or a fragment thereof. In some embodiments, the one or more first groups of stem cells comprise a nucleic acid sequence at least about 80% (at least about 80%, about 85%, about 90%, about 95%, about 98%, or about 99%) identical to SEQ ID NO: 2 or a fragment thereof. In some embodiments, the second group of stem cells comprise a nucleic acid sequence at least 80% (at least about 80%, about 85%, about 90%, about 95%, about 98%, or about 99%) identical to SEQ ID NO: 1 or a fragment thereof.

In some embodiments, the second group of stem cells do not have the inducible transgene encoding the GATA6 polypeptide. Accordingly, in those embodiments, the expression levels of the GATA6 polypeptide in the second group of stem cells are lower than expression levels of the GATA6 polypeptide in the first group of stem cells.

In some embodiments, step c) comprises mixing the one or more first groups of stem cells and the second group of stem cells at ratio of about 1:20 to about 1:2, preferably about 1:10 to about 1:3, or preferably about 1:10 to about 1:4.

In some embodiments, the mixture of the first group of stem cells and the second group of stem cells are placed on a surface (e.g., a flat surface or a non-flat surface). In some embodiments, the stem cells form a single layer of cells on the surface. The cells are then cultured on the surface for at least 7 days (e.g., at least 7 days, 8 days, 9 days, 10 days, 11 days, 12 days, 13 days, 14 days, 15 days, 18 days, 20 days, 25 days, or 30 days) or for about 4 to 10 days, about 6 to 12 days, about to 14 days, about 10 to 16 days, about 12 to 18 days, about 14 to 20 days, about 16 to 22 days, to 24 days, about 20 to 28 days, 24 to 30 days, or 25 to 35 days, thereby producing the cellular mass comprising one or more embryonic cell structures and one or more extraembryonic cell structures or one or more hematopoietic stem cells. The process of generating the cellular mass is self-organized and self-timed. The cellular mass can be generated from an initial randomly mixed state wherein the cells of step c) reproducibly form the one or more embryonic cell structure or extraembryonic cell structures described herein with or without additional reagents, patterning cues, growth factors, etc. added to the culture system. The one or more embryonic cell structure or extraembryonic cell structures can form on a consistent and reproducible schedule after induction of the circuit without additional reagents, patterning cues, growth factors, etc. added to the culture system. In some embodiments, step d) of the method of any preceding aspect does not require changing cell culture media or adding reagents during the process of producing the cellular mass. In some embodiments, step d) of the method of any preceding aspect further comprises changing cell culture media or adding reagents during the process of producing the cellular mass. In some embodiments, the generated cellular mass comprises one or more of a bilaminar disc-like structure, an amnion-like domain, a primitive streak-like domain, and a yolk sac domain. In some embodiments, the embryonic cell structure comprises an increased level of an octamer-binding transcription factor 4 (OCT4) polypeptide and a decreased level of a GATA4 polypeptide as compared to a reference control. In some embodiments, the extraembryonic cell structure comprises an increased level of the GATA6 polypeptide as compared to a reference control.

“OCT4” refers herein to a polypeptide that, in humans, is encoded by the POU5F1 gene. In some embodiments, the OCT4 polypeptide is that identified in one or more publicly available databases as follows: HGNC: 9221, NCBI Entrez Gene: 5460, Ensembl: ENSG00000204531, OMIM@: 164177, UniProtKB/Swiss-Prot: Q01860. In some embodiments, the OCT4 polypeptide comprises the sequence of SEQ ID NO: 12, or a polypeptide sequence having at or greater than about 80%, about 85%, about 90%, about 95%, or about 98% homology with SEQ ID NO: 12, or a polypeptide comprising a portion of SEQ ID NO: 12. The OCT4 polypeptide of SEQ ID NO: 12 may represent an immature or pre-processed form of mature OCT4, and accordingly, included herein are mature or processed portions of the OCT4 polypeptide in SEQ ID NO: 12.

“GATA4” refers herein to a polypeptide that, in humans, is encoded by the GATA4 gene. In some embodiments, the GATA4 polypeptide is that identified in one or more publicly available databases as follows: HGNC: 4173, NCBI Entrez Gene: 2626, Ensembl: ENSG00000136574, OMIM®: 600576, UniProtKB/Swiss-Prot: P43694. In some embodiments, the GATA4 polypeptide comprises the sequence of SEQ ID NO: 13, or a polypeptide sequence having at or greater than about 80%, about 85%, about 90%, about 95%, or about 98% homology with SEQ ID NO: 13, or a polypeptide comprising a portion of SEQ ID NO: 13. The GATA4 polypeptide of SEQ ID NO: 13 may represent an immature or pre-processed form of mature GATA4, and accordingly, included herein are mature or processed portions of the GATA4 polypeptide in SEQ ID NO: 13.

In some embodiments, the cellular mass of any preceding aspects comprises one or more of a bilaminar disc-like structure, an amnion-like domain, a primitive streak-like domain, and a yolk sac domain. In some embodiments, the amnion-like domain comprises an increased level of one or more of bone morphogenetic protein 4 (BMP4), bone morphogenetic protein receptor type 1A (BMPR1A), distal-less homeobox 5 (DLX5), follistatin like 1 (FSTL1), inhibitor of DNA binding 1 (ID1), lymphoid enhancer binding factor 1 (LEF1), msh homeobox (MSX)1, MSX2, SMAD family member 1 (SMAD1), SMAD specific e3 ubiquitin protein ligase 2 (SMURF2), and ISL LIM Homeobox 1 (ISL1).

In some embodiments, the cell mass comprises one or more neural features (including, for example, increased levels of one or more of a cerberus 1 (CER1) polypeptide and a left-right determination factor 1 (LEFTY1) polypeptide).

In some embodiments, the method of any preceding aspect further comprises contacting the one or more first group of stem cells, the second group of stem cells, and/or the third group of stem cells with an agent (e.g., an agent that improves the signature of trophoblast cells differentiated from the embryonic compartment of the system). In some embodiments, the agent is SB431542.

The cellular mass generated by the methods disclosed herein can be used for evaluating, testing, and/or screening agents (e.g., therapeutic agents) that modulate embryogenesis or hematopoietic differentiation, comprising generating a cellular mass that comprises one or more embryonic cell structures and one or more extraembryonic cell structures by the method of any preceding aspects; and contacting the cellular mass with the agent. Accordingly, in some aspects, disclosed herein is a method of evaluating an agent that modulates embryogenesis or hematopoietic differentiation, said method comprising
generating a cellular mass that comprises one or more embryonic cell structures and one or more extraembryonic cell structures by the method of any preceding aspect;
contacting the cellular mass with the agent, and
determining the effect of the agent on the cellular mass as compared to a control.

In some embodiments, the method further comprises determining the biomarkers related to embryogenesis or hematopoietic differentiation. The biomarkers related to embryogenesis or hematopoietic differentiation are well-known in the art.

Also included herein is a method of evaluating embryogenesis modulating agent or an agent that modulates hematopoietic differentiation, said method comprising
a) obtaining one or more of a first group of stem cells comprising an inducible transgene encoding a GATA binding protein 6 (GATA6) polypeptide;
b) contacting the one or more first group of stem cells with an inducing agent that induces an expression of the transgene;
c) mixing the one or more first group of stem cells of step b) with a second group of stem cells; and
d) culturing the mixture of cells of step c) on a surface for at least 7 days with the embryogenesis modulating agent, thereby producing a cellular mass and analyzing the cellular mass as compared to a control.

In some embodiments, the method further comprises determining the biomarkers related to embryogenesis or hematopoietic differentiation. The biomarkers related to embryogenesis or hematopoietic differentiation are well-known in the art. In some embodiments, the agent is a therapeutic agent. In some embodiments, the agent is a toxin.

Also disclosed herein is a cellular mass produced by the method of any preceding aspect, wherein the cellular mass comprises one or more embryonic cell structures and one or more extraembryonic cell structures.

Also disclosed herein is a method of generating a human primordial germ cell-like cell, comprising
a) obtaining one or more of a first group of stem cells comprising an inducible transgene encoding a GATA binding protein 6 (GATA6) polypeptide;
b) contacting the one or more first groups of stem cells with an inducing agent that induces an expression of the transgene;
c) mixing the one or more first groups of stem cells of step b) with a second group of stem cells;
d) culturing the mixture of cells of step c) on a surface for at least 3 days, thereby producing the cellular mass;
e) isolating the human primordial germ cell-like cell from the cellular mass.

The human primordial germ cell-like cell can be isolated from the cellular mass at day 2, 3, 4, 5, 6, 7, 8, 9, or 10 post culturing the mixture of stem cells on the surface. In some embodiments, the generated human primordial germ cell-like cell has an increased expression of one or more of CD38, BLIMP1, and AP2γ as compared to a control.

Also disclosed herein is a human primordial germ cell-like cell produced by the method of any preceding aspect, wherein the human primordial germ cell-like cell has an increased expression of one or more of CD38, BLIMP1, and AP2γ as compared to a control.

Also disclosed herein is a method of treating a fertility disorder in a subject in need, comprising
generating a cellular mass that comprises one or more embryonic cell structures and one or more extraembryonic cell structures by the method of any preceding aspect; and
transplanting one or more embryonic cell structures and/or one or more extraembryonic cell structures of the cellular mass into the subject.

In some embodiments, the stem cell that is used for generating the cellular mass is derived from the subject. In some embodiments, the stem cell that is used for generating the cellular mass is an engineered stem cell.

In some embodiments, the generated yolk sac has hematopoietic characteristics, including erythrogenesis and myelogenesis. Accordingly, in some aspects, disclosed herein is a method of
generating a hematopoietic stem cell, comprising generating a cellular mass that comprises one or more embryonic cell structures and one or more extraembryonic cell structures by the method of any preceding aspect; and
isolating the hematopoietic stem cell from the cellular mass.

The hematopoietic stem cell can be isolated from the cellular mass at day 7, 8, 9, 10, 11, 12, 13, 14, 15, 16, 17, 18, 19, 20, 21, 22, 23, 24, 25, 26, 27, 28, 29, or 30, or at least 7 days (e.g., at least 7 days, 8 days, 9 days, 10 days, 11 days, 12 days, 13 days, 14 days, 15 days, 18 days, 20 days, 25 days, or 30 days) or about 4 to 10 days, about 6 to 12 days, about 8 to 14 days, about 10 to 16 days, about 12 to 18 days, about 14 to 20 days, about 16 to 22 days, 18 to 24 days, about 20 to 28 days, 24 to 30 days, or 25 to 35 days post culturing the mixture of stem cells on the surface. In some embodiments, the generated hematopoietic stem cell has an increased expression of one or more of CD3, CD19, CD33, CD56, CD45, CD11b, and CD34 as compared to a control.

Also disclosed herein is a method of treating a hematopoietic disorder in a subject in need, comprising
generating a cellular mass that comprises one or more embryonic cell structures and one or more extraembryonic cell structures by the method of any preceding aspect;
isolating the hematopoietic stem cell from the cellular mass; and
transplanting the hematopoietic stem cell into the subject.

In some embodiments, the stem cell that is used for generating the hematopoietic stem cell is derived from the subject. In some embodiments, the stem cell that is used for generating the hematopoietic stem cell is an engineered stem cell.

Examples of hematopoietic disorders include but are not limited to, megaloblastic (pernicious) anemia, sickle cell disease, thalassemia, leukemia, and bone marrow dysplasia syndrome.

Also disclosed herein is a method of generating an extracellular matrix, comprising
a) obtaining one or more of a first group of stem cells comprising an inducible transgene encoding a GATA binding protein 6 (GATA6) polypeptide;
b) contacting the one or more first groups of stem cells with an inducing agent that induces an expression of the transgene;
c) mixing the one or more first groups of stem cells of step b) with a second group of stem cells;
d) culturing the mixture of cells of step c) on a surface thereby producing a cellular mass; and
e) isolating the extracellular matrix from the cellular mass of step d).

In some embodiments, the extracellular matrix can be isolated from cellular mass at day 2, 3, 4, 5, 6, 7, 8, 9, 10, 11, 12, 13, 14, 15, 16, 17, 18, 19, 20, 21, 22, 23, 24, 25, 26, 27, 28, 29, or 30 post culturing the mixture of stem cells on the surface. In some embodiments, the generated extracellular matrix is substantially composed of one or more of LAMA1, LAMA2, COL4A1, and COL2A1 as compared to a control.

The extracellular matrix can be the physical scaffolding and/or protein architecture that the cells create in order to establish and maintain the physical form of the cellular mass and its structure(s). The extracellular matrix can be isolated by killing and washing away the cells of the cellular mass but leaving the scaffolding intact. The decellularized extracellular matrix can be recellularized for transplants. The extracellular matrix can then be used to culture entirely different cell types that may benefit from having the existing structures patterned by the original iPSCs (cavities, vessel areas, etc). These cells, which can be any cell type of interest, inhabit this extracellular matrix and be cultured in different media conditions.

Accordingly, in some aspects, disclosed herein is an in vitro or ex vivo culture system comprising the extracellular matrix generated by the method of any preceding aspect.

It should be understood and herein contemplated that the transgenes, the polynucleotides, or the polypeptides (e.g., GATA6 and ETV2) can be delivered to the stem cells or a subject in need through any known route of delivery, including, for example, transfection-based delivery of mRNA of GATA6 or ETV2, or protein delivery of GATA6 or ETV2. DNA plasmid vectors can also be delivered to engineering cells with a stable integrated gene circuit introduced via piggybac transposase. In some examples, the cells can be generated in vitro and then be transplanted in vivo. After transplantation, the cells can be induced in vivo.

### EXAMPLES

The following examples are set forth below to illustrate the compositions, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1. Methods

Ethics regarding development of iDiscoids. This research was approved and performed under the oversight of the University of Pittsburgh Human Stem Cell Research Oversight Committee (hSCRO #20-004) and follows the 2021 ISSCR Guidelines on Ethical Standards for Stem Cell Embryo Models. To this end, iDiscoids do not involve human embryonic stem cells and are generated from human iPSCs that are derived from human somatic cells such as fibroblasts. iDiscoids are attached to a cell culture dish, lacking an extra-embryonic trophectoderm tissue critical for full integrated embryo development and implantation in the uterine cavity. The yolk sac cavity in iDiscoids are not closed, and the tissue cannot be harvested for any implantation without substantial disruption of their structures. TBXT⁺ posterior domains were observed during the study but were not sustained during the development of our system. These features collectively restrict the ability of this model to undergo the full integrated development of a human embryo in vitro and/or implant to support further development of a conceptus in vivo.

Cell Culture. All cells and tissues were cultured in a humidified incubator at 37° C. and 5% CO₂. PGP1 and PGP9 were used in this study. The hiPSC lines were cultivated under sterile conditions in mTeSR-1 (Stem Cell Technologies, Vancouver) changed daily. Tissue culture plates were coated for 1 hour at room temperature with BD ES-qualified Matrigel (BD Biosciences) diluted according to the manufacturer's instructions in ice cold DMEM/F-12 with 15 mM HEPES medium (Thermo Scientific). Routine passaging was performed by incubating hiPSC colonies for 5 minutes in Accutase (Sigma) at 37° C., collecting the suspension and adding 5 mL DMEM/F-12 medium containing 10 mM Y-27632, centrifuging at 300 g for 5 minutes, and resuspending in DMEM/F-12 supplemented with 10 mM Y-27632 for counting. Cells were seeded at a cell density of 25,000 cells per cm².

GATA6-Engineered Cell Line Generation. rtTA expressing hiPSCs previously generated (Guye, P. et al. 2016) were transfected using Lipofectamine 3000 (Thermo Fisher Scientific) with Super PiggyBac Transposase (System Biosciences) and the PiggyBac transposon vector with hGATA6-2A-EGFP under control of the tetracycline responsive element promoter. Transfected cells were selected by adding 0.5 mg/mL puromycin to the mTeSR1 maintenance medium.

Generation of iDiscoid. The GATA6-engineered hiPSCs were seeded at a ratio of 4:1 with rtTA expressing hiPSCs either containing or lacking an mKate reporter gene at a total density of 25,000 cells per cm² in mTeSR-1 supplemented with 10 mM Y-27632. The next day, the medium was changed to mTeSR-1 with 1 mg/mL doxycycline to induce expression of the GATA6 transgene and replaced daily for up to 5 days.

iDiscoid Cryostorage. Uninduced iDiscoid cells were incubated with Dispase at 70% confluence for 10 minutes, or until visible lifting of colony edges. Cells were washed twice with DMEM/F-12, then colonies were manually scraped from the plate. Colonies were centrifuged at 300 g for 5 minutes, and then were resuspended in Cryostor 10. Cells were cooled at −80° C. for 24 hours prior to transfer to liquid nitrogen for long-term storage. Cells were stored in liquid nitrogen for at least 24 hours prior to defrosting.

Signaling Pathway Inhibition. BMP4 signaling was inhibited via application of 3 μM Dorsomorphin into the normal media at day 3 of iDiscoid culture. NODAL signaling was inhibited via application of 10 μM SB431542 beginning at day 2 of iDiscoid culture. CXCR4 signaling was inhibited via application of 1p M AMD3465 beginning at day 0 of iDiscoid culture.

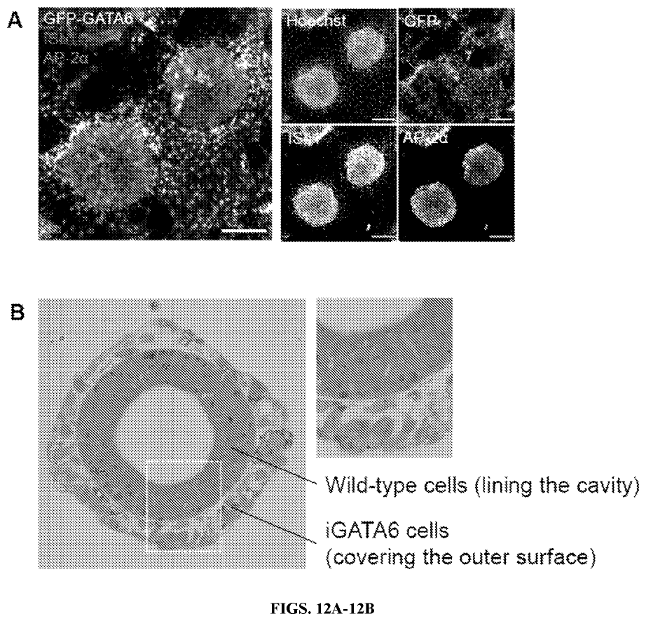

Staining on glass coverslips. Cells were grown on Matrigel-coated 8 mm diameter circular glass coverslips or on Mattek 35 mm coverslip bottom dishes. Cultures were fixed for 10 minutes in 4% paraformaldehyde (Electron Microscopy Sciences) at room temperature. For toluidine blue staining in FIG. 12B, cells were fixed for 1 hour in 2.5% glutaraldehyde in 0.01M PBS, pH 7.4, at room temperature. Coverslips were then washed three times with PBS followed by 15 minutes permeabilization with 0.2% Triton X-100 in PBS. Subsequently the coverslips were washed three times in washing buffer (0.05% Tween-20 in PBS) for 5 minutes and blocked for 20 minutes in 200 ml wash buffer plus 5% normal donkey serum (Jackson ImmunoResearch Laboratories). The primary antibodies were diluted in 5% normal donkey serum in PBS and incubated with the tissues hour at room temperature followed by three washes in wash buffer for 5 minutes each. The secondary antibodies were diluted in 5% normal donkey serum in PBS and incubated with the tissues 1 hour at room temperature followed by three washes in wash buffer for 5 minutes each. Afterwards, the 8 mm coverslips were mounted on microscopy glass slides using ProLong Glass Antifade (Life Technologies), cured overnight at room temperature and then sealed with nail polish. Coverslips in Mattek dishes were stored in PBS at 4° C. for 3D imaging prior to mounting.

Image Acquisition and Processing. Images were acquired using the EVOS M700 automated scanning microscope, Leica SP8 confocal microscope, or Nikon A1 Confocal microscope and processed using ImageJ software (NIH). Any contrast adjustments were made in individual channels and applied evenly across the whole image in that channel. Contrast and color balance for color images was applied evenly across the whole image. 3D reconstructions were generated using the Nikon A1 confocal microscope to generate z-stacks spanning ˜100 um deep into the tissues and using ImageJ or Imaris (Bitplane) to construct a 3D volume from the stacks.

Time lapse Imaging. iGATA6 cells were seeded at 25,000 cells per cm² in a Cellstar 24-well tissue culture treated plate coated w37ndicatiigel, then induced with 1,000 ng ml⁻¹ Dox. After induction, the cells were placed under an Incucyte S3 live cell imaging platform at 37C and 5% CO₂. Images were taken every 30 min for 3 days.

Analysis of Wild-Type Cluster Areas and Radial Expression. Tiled whole-coverslip images were cropped to a central 9000 μm² image for analysis. Wild-type compartment analysis was performed using an in-house Matlab pipeline. In brief, wild-type compartments were detected programmatically via thresholding the nuclear dye channel for areas of high cell density. Maximum individual compartment area and average cell size were defined manually. Compartments were filtered to remove those with areas close to the defined cell area via the following equation:

$$lowerSizeLimit = \frac{max CompartmentArea}{{10}^{( \frac{( {log}_{10} max CompartmentArea - {log}_{10} singleCellArea )}{2} )}}$$

For compartment counting and wild-type area analysis, characteristics of each compartment were recorded using the regionprops function in Matlab. Additionally, the distance to the nearest wild-type compartment was recorded.

For radial analysis of marker expression outside each wild-type compartment, an additional filtering step was applied. Compartments were eliminated from analysis if their proximity to nearby compartments or the image border would cause the maximum radial analysis distance to overlap another compartment or the image border. Radial analysis was performed on the remaining compartments via drawing a line perpendicularly outward from each pixel of the compartment perimeter to the defined maximum analysis distance and recording the intensity value of each marker at each point within this range. If a line was drawn such that it would intersect the compartment (e.g. drawing from the inner edge of a concave shape), those values were skipped. Intensity values at each distance were averaged to create final per-compartment expression curves.

Analysis of marker expression inward from the compartment perimeter was performed in a similar fashion, except that lines were drawn towards each compartment's centroid rather than perpendicularly from the compartment edge. Coverage of WT clusters was evaluated via widefield images taken of the entire coverslip; areas in which EGFP expression was observed inside of WT clusters greater than 10% of the cluster radius was counted as being covered. Presence or absence of lumens/cavities was evaluated manually, and was detected via the presence of visible rings of PODXL expression within wild-type compartments.

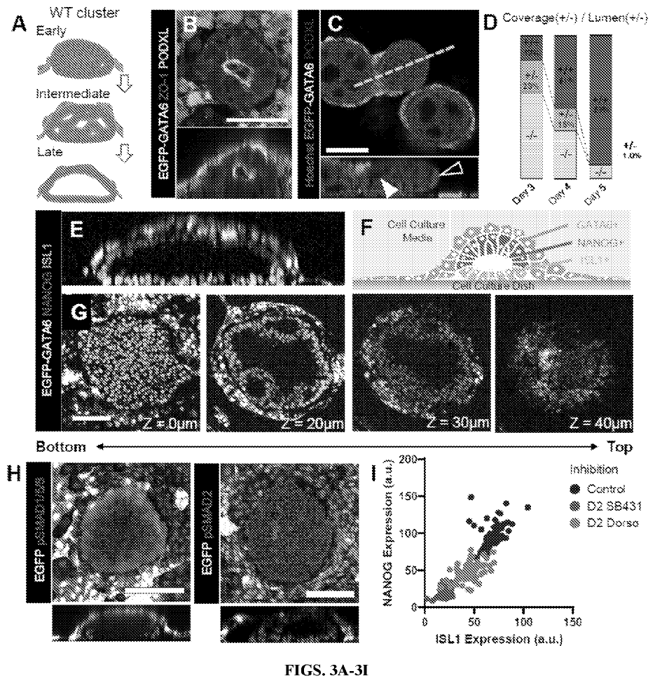

Analysis of Wild-Type Cluster Features. Radial averages of marker expression intensity from the GFP image channel were used to define iGATA6 coverage. Following radial analysis, compartments of complete, partial, and no coverage were manually identified, and the average radial intensity curves of those compartments were used to categorize other compartments on a per-image set basis. Both complete and partial coverage were considered to be positive coverage for FIG. 3G.

Wild-type clusters were categorized into “early”, “intermediate”, or “late” categories based on the state and size of lumens within each cluster. “Early” clusters showed no or very minimal inward polarization marked by PODXL following establishment of contact with GATA6-expressing cells. “Intermediate” clusters were marked by the expansion of small lumens and the presence of multiple lumens within the cluster. “Late” clusters were marked by a single contiguous lumen centered within the cluster.

Enzyme-linked Immunosorbent Assays (ELISA). Samples were assayed for AFP, APOA1, and BMP4 using commercially available ELISA kits (abcam). Sample dilutions were optimized to attain detection in the linear range of the standard curves for each individual assay.

10× Genomics Sample Preparation for Next-generation Sequencing. Samples were prepared as described by the 10× Genomics Single Cell 30 v2 Reagent Kit user guide. Day 5 iDiscoids initially mixed at a ratio of 10:1 iGATA6:WT-iPSCs were acquired in single cell suspension by incubation with trypsin for 10 minutes at 37° C., followed by gentle pipetting using a serological pipette to dislodge and dissociate aggregates. Two washes in PBS−/−+0.04% BSA were performed with the cells re-suspended at a final concentration of 1000 cells/mL in PBS−/−+0.04% BSA. A live cell count was performed with a hemocytometer using Trypan Blue to identify dead cells. Following counting, cells and 10× Genomics reagents were loaded into the single cell cassette, with a target of 6000 single cells for analysis, accounting for predicted cell loss and doublets as laid out in the user guide for the Chromium Single-Cell 3′ Reagent Kit (10× Genomics). After generation of GEMs, the cDNA library was prepared by ASU Genomics Core staff following the appropriate steps determined by the 10× Genomics user guide. Libraries were sent to Novogene for sequencing by an Illumina HiSeq X for an intended read depth of 100,000 reads per cell with 150 bp paired end reads. Our downstream analysis from the sequencing data estimates the actual number of cells sequenced was between 5000 and 7000 which would yield >80 k reads per cell. The 10× Genomics CellRanger pipeline was used to align reads to the reference genome (GRCh38.84) appended with transgene sequences, to assign reads to individual cells, and to estimate gene expression based on UMI counts (Zheng, G. X. et al. 2017).

Single cell data was excluded based on high mitochondrial genome transcript ratio and either high or low feature or UMI counts. Genes with UMI counts in fewer than 5 cells were removed from consideration. For scRNA-Seq data processing and cluster analysis using Seurat (Butler, A. et al., 2018; Satija, R. et al., 215), this study used the following general standardized pipeline for processing of the Cell Ranger output: Normalization, feature selection, scaling (including cell cycle regression), principal component analysis (PCA), and clustering. Elbow plots and permuted p-values were used to assist in determining the optimal number of principal components (PCs) needed to summarize the datasets without losing a significant amount of variation. The quality of a range of clustering resolution values were assessed using enrichment of cluster marker genes (genes differentially upregulated in a given cluster relative to all other clusters) with embryo cell type-specific genes. As a quality check, PC and resolution metrics were modulated to yield fewer or additional clusters to confirm that chosen parameters resulted in the most biologically relevant clustering. Visualization was achieved by the use of tSNE plots identifying cells, clusters, and selected gene expression in each cell, as well as heatmaps and violin plots showing the expression level of genes by cluster.

Statistical similarity quantification between lists of marker genes. The similarity between two lists of marker genes A and B is quantified by using the Hypergeometric test for overrepresentation. This is equivalent to a one-tailed Fisher's Exact Test.

Integration with published datasets and label projection. To directly compare cell type annotations between our human cell line data and published non-human data, we follow a data integration protocol similar to (Minn, K. T. et al. 219). First we select the subset of our human cell line genes which have orthologs in the mouse and NHP data. Using these orthologous gene mappings, we use a subset of the genes from both datasets which are contained in the orthologous set of genes. Specifically, if our human cell line data was contained in a count matrix H of dimensions (n cells, h genes) and the non-human data was contained in a count matrix M of dimensions (k cells, m genes), then we subset both datasets to respective matrices H′ (n, g) and M′ (k, g) so that both contain the same number of genes. Further, these g genes are orthologous between H′ and M′ and are ordered so that they correspond to each other between the datasets. We note that orthologous gene mappings can be many-to-one, so we duplicate genes and keep all of these orthologs.

After obtaining H′ and M′, we then proceed by integrating the datasets using the FindIntegrationAnchors and IntegrateData functions in the Seurat package (version 3) (Stuart, T. et al. 2019). We followed the guide for doing this analysis (satijalab.org/seurat/v3.1/integration.html #standard-workflow), which only considers the 2000 most highly variable genes out of the g genes. This then results in a new integrated data matrix X of shape (n+m, 2000).

To project the cell type labels from the E16-19 data onto our human cell line data, we begin by computing the first 30 Principal Components of this X data matrix (yielding a matrix X′ of shape (n+m, 30)). We then use only the labeled data in this reduced dimension (a matrix of shape (m, 30)), to train a k-Nearest Neighbor classifier (we use k=5). We then apply this classifier to predict the cell type labels of the (n, 30) human cell line subset of the data. These predicted labels are final “projected labels.”

In vitro Colony-Forming Unit Assays. Cells were dissociated into single cell solution using accutase (StemCell Technologies). CD34+ cells were purified using from a portion of the cells using the CD34 Microbead Kit Ultrapure and LS columns (Miltenyi Biotec) according to the manufacturer's instructions. Colony forming unit assays were performed using methylcellulose containing media (MethoCult H4434 Enriched and MethoCult SF H4636, StemCell Technologies) according to the manufacturer instructions. After 14 days in culture, plates were scored visually for CFU-multilineage colonies and subsequently analyzed immunophenotypically via flow cytometry.

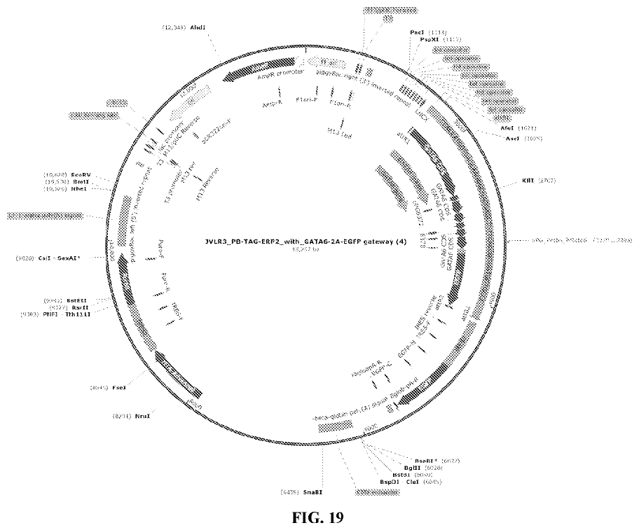

Flow Cytometry Analysis. Input and output cells to the colony forming unit assays were analyzed immunophenotypically via flow cytometry. Cells were stained with human anti-CD235a, anti-CD71, anti-CD45, anti-CD11b, and anti-CD34 antibodies (Biolegend) for 30 minutes on ice. Cells were analyzed using an LSR II flow cytometer (BD Biosciences) using 7-AAD (ThermoFisher Scientific) as a dead stain. Data analysis was performed using FlowJo software. An example of gating strategy can be found in FIG. 18. To prepare samples, iDiscoids were developed in 4-6 wells of a 6 well plate, and samples were pooled prior to CD34+ cell isolation using the MACS separation system (Stemcell Tech).

### Example 2. Modelling Human Post-Implantation Development Via Extra-Embryonic Niche Engineering

Implantation of human embryo commences a critical developmental stage that comprises profound morphogenetic alteration of embryonic and extraembryonic tissues, axis formation and gastrulation events. The mechanistic knowledge of this window of human life remains limited due to restricted access to natural healthy samples for both technical and ethical reasons. Additionally, human stem cell models of early post-implantation with both embryonic and extra-embryonic morphogenesis are lacking. Here, this study presents iDiscoid, produced from human induced pluripotent stem cells via engineered synthetic gene circuits. iDiscoids show reciprocal co-development of human embryonic tissue and engineered extra-embryonic niche. They exhibit unanticipated self-organization and tissue boundary formation that recapitulates yolk sac-like tissue specification with hematopoietic characteristics, the formation of bilaminar disc-like embryonic morphology, the development of the amniotic-like cavity, and acquisition of a posterior axis. iDiscoids offer an easy-to-use, high-throughput, reproducible and scalable platform to probe multifaceted aspects of human early post-implantation development. This system provides a tractable human model for drug testing, developmental toxicology, and disease modelling.

This study presents a human stem cell-derived post-implantation embryo model, dubbed “iDiscoid”, that overcomes several of these limitations. iDiscoid engineering leverages a synthetic biology approach to pre-program a population of human induced pluripotent stem cells (hiPSCs) with inducible genetic circuits. Upon activation of genetic circuits, an extra-embryonic niche is formed that directs self-organization of the adjacent wild-type hiPSCs. Using iDiscoids, this study demonstrates development of human bilaminar disc-like structures, lumenogenesis, formation of amnion-like tissue, symmetry breaking leading to posterior axis specification, and yolk sac specification with hematopoietic characteristics.

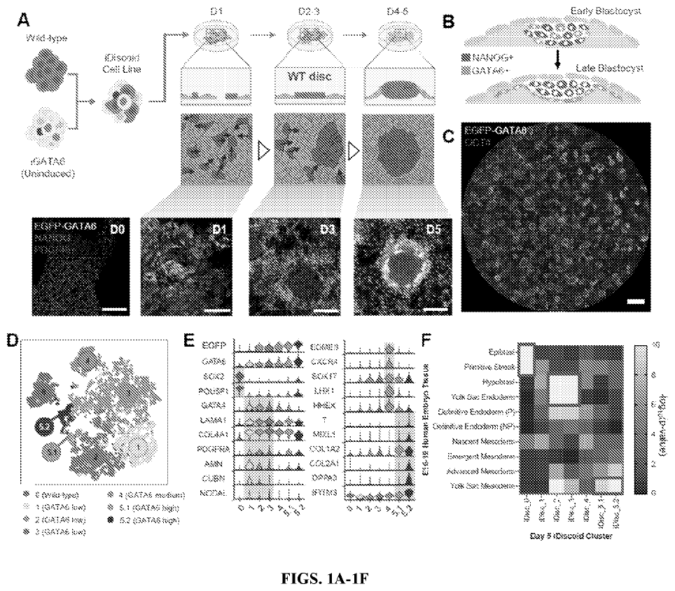

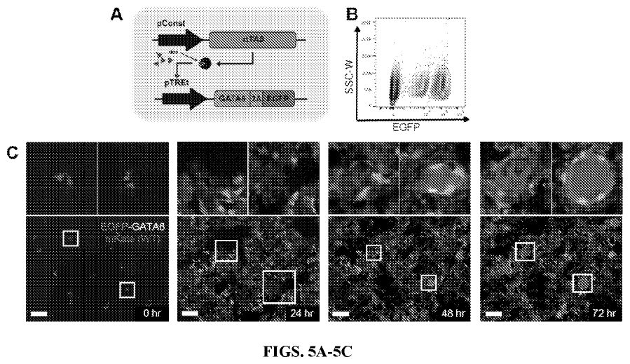

Engineering co-development of embryonic and extra-embryonic tissues. A hiPSC cell line was engineered with an inducible gene circuit that expresses the human GATA6 transcription factor after the addition of a small molecule, Doxycycline (Dox) (henceforth referred to as iGATA6) (FIG. 1A, FIG. 5A). These iGATA6 cells were engineered to express different levels of GATA6 upon circuit induction, distinguishable by expression of the reporter enhanced green fluorescent protein (EGFP) (FIG. 5B). The iGATA6-hiPSCs were mixed with wild-type hiPSCs (WT), and the mixed population of cells were seeded onto standard culture plates in 2D (FIG. 1A).

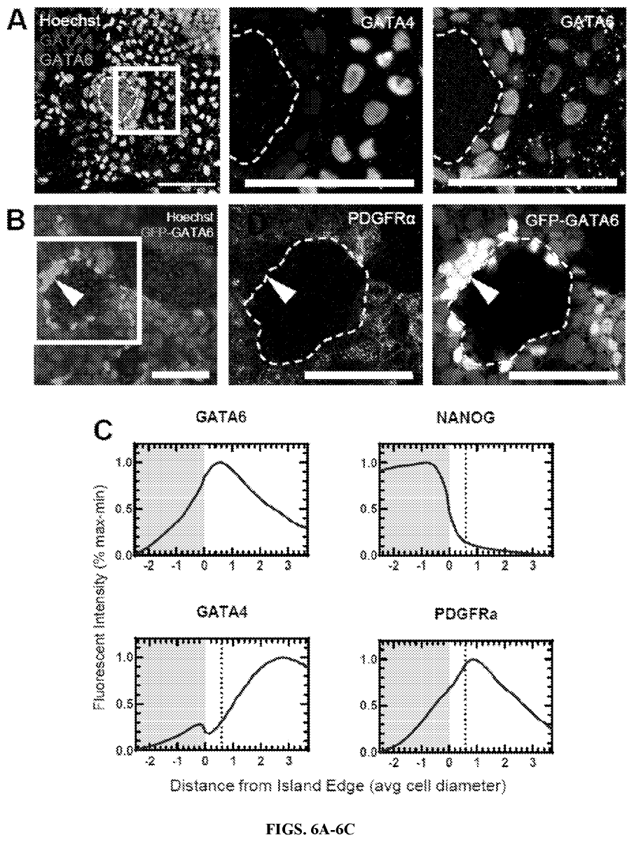

The initial state of the system mirrors the salt-and-pepper distribution of cells of the hypoblast (GATA6⁺), and epiblast (NANOG⁺) in the inner cell mass of embryonic day (E)6 stage embryo in vivo (FIG. 1B). After treatment with Dox, the iGATA6 cells upregulate EGFP and exhibit loss of the pluripotency marker NANOG (FIG. 1A). During the first 72 hours, from an initial state of random distribution, induced iGATA6 cells and WT cells sort out from one another. After 72 hours, WT cells form disc-shaped clusters (WT discs) (FIGS. 1A, 1C, and FIG. 5C). At the same time, iGATA6 cells self-organize, resulting in iGATA6 cells with low levels of exogenous GATA6 induction (lower EGFP) sorting away from the WT discs and expressing characteristic markers of primitive endoderm, including PDGFRα and GATA4 (FIGS. 6A-6B). Cells expressing the highest levels of exogenous GATA6 (high EGFP) surrounding the WT discs lack expression of these markers, indicating a divergence in fate away from primitive endoderm (FIGS. 6A-6C).

Single cell RNA transcriptomics reveals embryonic and extraembryonic fate trajectories. To investigate the cellular identities of each iGATA6 population, this study performed single cell RNA sequencing (scRNAseq) analysis on 4,135 cells derived from day 5 cultures. This analysis identified the emergence of 5 cellular clusters, indicating the specification of distinct identities (FIGS. 1D-1E). Performing an unsupervised comparison of the transcriptomes with the E16-19 human embryo (Tyser, R. C. V. et al. 2020) revealed the acquisition of distinct post-implantation cellular fates in iDiscoid subpopulations (FIG. 1F). The three largest clusters (clusters 1, 2, 3) expressed the lowest average positive EGFP and GATA6 transcript counts; these clusters showed significant transcriptomic similarity to human post-implantation yolk sac endoderm and hypoblast (FIG. 1F). These clusters expressed putative lineage markers associated with human hypoblast, including LAMA1, GATA4, and PDGFRα. Two of the three clusters additionally expressed human hypoblast markers CUBN, AMN, and NODAL (FIG. 1E) (Cindrova-Davies, T. et al. 2017). The WT population (cluster 0) showed highly significant similarity to human post-implantation epiblast and primitive streak cells (FIG. 1F). These cells expressed pluripotency-associated factors SOX2 and OCT4 (POU5F1) and lacked GATA6 or EGFP transcripts (FIG. 1E).

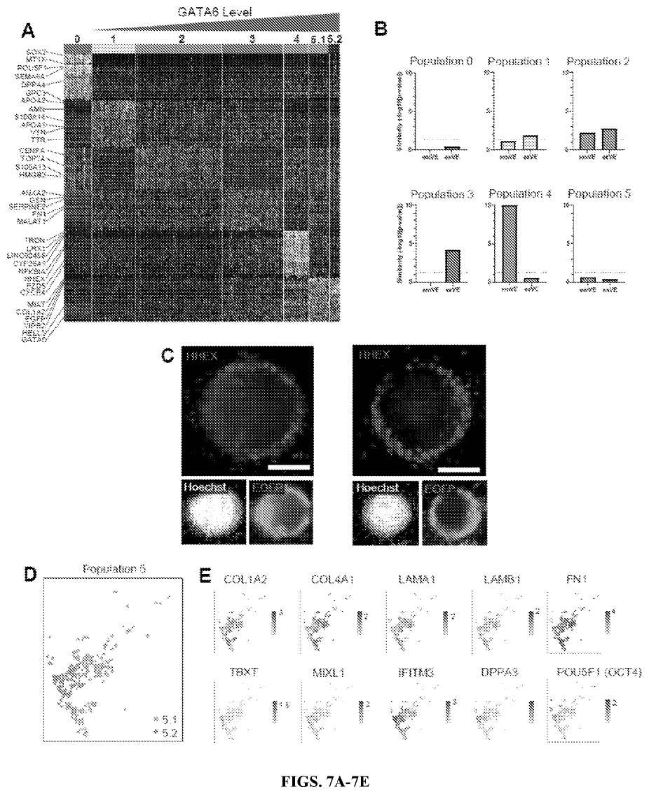

The cluster containing the second-highest average EGFP transcript expression (cluster 4), exhibits differential upregulation of genes responsible for regulation of the anterior hypoblast; LHX1, a significant activator of anterior endoderm programs in mouse and anterior hypoblast marker in human, HHEX, a marker of human anterior hypoblast, and CXCR4, a marker of E16-anterior endoderm (FIG. 1E, FIG. 7A) Tyser, R. C. V. et al. 2020; Costello, I. et al. 2015; Mole, M. A. et al. 2021. When compared with available datasets, it showed similarity to both hypoblast and definitive endoderm of E16-19 stage human embryo, and strong correlation with E6.5-E8.25 mouse embryonic visceral endoderm (emVE) (FIG. 1F, FIG. 7B). Immunofluorescence revealed that this population occupies the area adjacent to the WT disc (FIG. 7C).

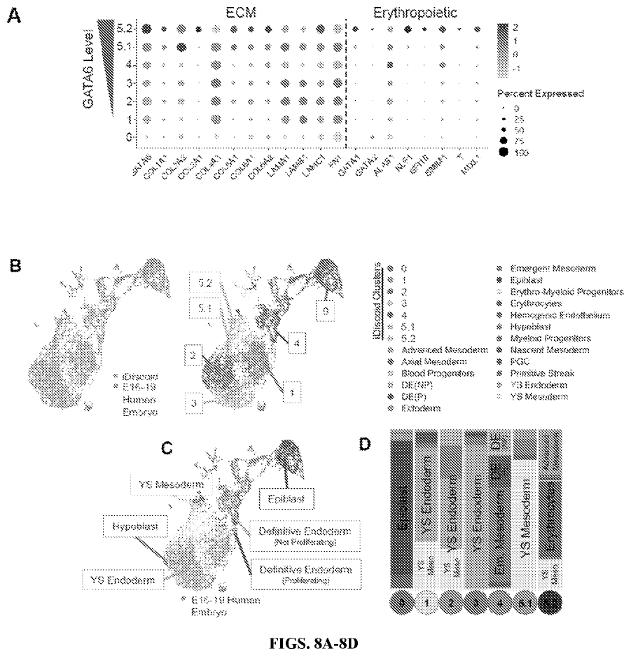

Analysis of the cluster with highest expression of GATA6 and EGFP reveals two distinct subpopulations, both with significant similarity to human yolk sac mesoderm (FIG. 1F, FIG. 7D). One (labeled 5.1) shows higher average expression of ECM proteins (COL1A2, COL4A1, LAMA1, LAMB1, FN1); the other subcluster exhibits markers of hematopoietic tissue within the murine allantois (TBXT, MIXL1, IFITM3 (Fragilis), DPPA3 (Stella), OCT4) and highest average transcript number for both GATA6 and EGFP (labeled 5.2) (FIG. 1E, FIG. 7E, 8A) (Tyser, R. C. V. et al. 2020; Ross, C. & Boroviak, T. E. 2020; Wolfe, A. D., Rodriguez, 2017; Downs, K. M., 2009; Downs, K. M. 2020). Cluster 5.2 uniquely contains TBXT and MIXL1 co-expressing cells and expresses erythropoietic markers GATA1, GATA2, ALAS2, KLF1, GFI1B, and SMIM1 (FIG. 1F, FIG. 7E, FIG. 8A) (Barbarani, G. 2019; Zhang, Y. et al. 2017; Cvejic, A. et al. 2013; Lancrin, C. et al. 2012; Bruveris, F. F. et al. 2020). Additional analysis via label transfer between populations additionally confirmed the separate identities of these subpopulations, including a substantial alignment to yolk sac-derived erythrocytes (47%) for cluster 5.2 (FIGS. 8B-8D).

Specification of yolk sac mesoderm-like cells with hematopoietic potential. This study further examined the observed yolk sac signatures and their associated functional characteristics. Human yolk sac, comprised of extra-embryonic endoderm and mesoderm, is a major source of serum proteins and lipids, and is the niche for emergence of early primitive and pro-definitive hematopoiesis around E16-18 of human development (Ross, C. & Boroviak, T. E et al., 2020; Nakazawa, F. et al., 2011; Palis, J. & Yoder, M. C. 2001). Human yolk sac mesoderm has been suggested to differentiate from yolk sac endoderm and emerges in the form of spindle-shaped cells (Ross, C. & Boroviak, T. E et al., 2020; Enders, A. C. & King, B. F. 1988). As human embryos are not amenable to research, the use of model systems offers a means to identify molecular and cellular mechanisms of yolk sac tissue. Importantly, however, yolk sac tissue morphogenesis and its hematopoietic functions have not been captured in past stem cell-based models of human embryonic development.

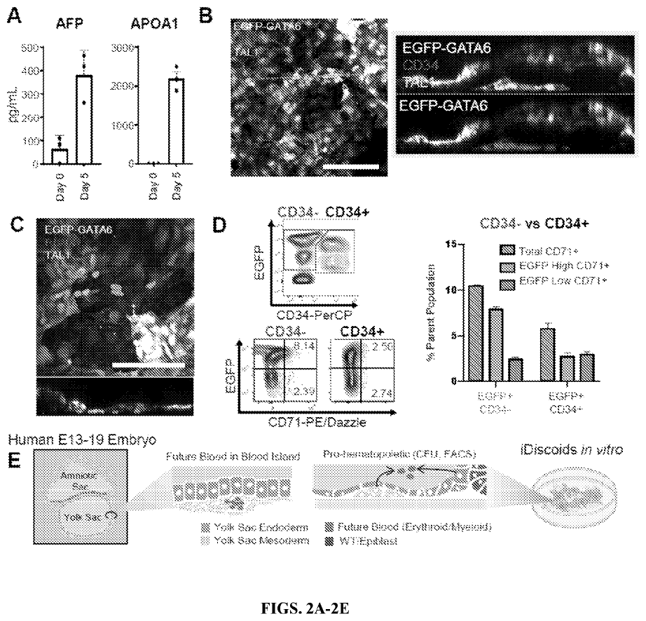

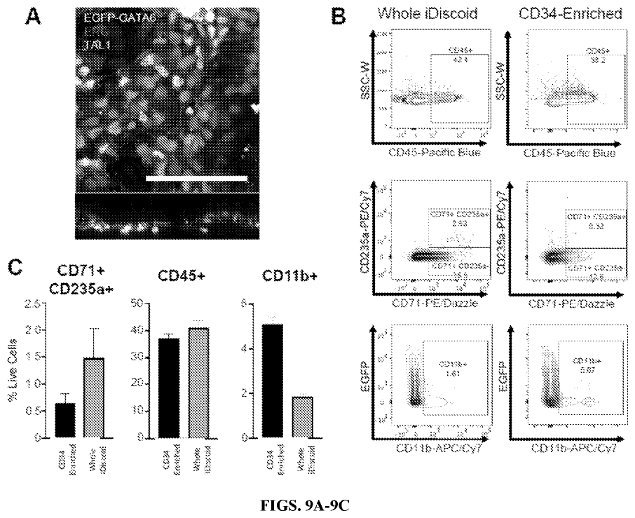

The experiment first confirmed high levels of secreted AFP, a major serum protein associated with primitive erythropoiesis (An, J. et al., 2019), and APOA1, an apolipoprotein with roles in monocyte proliferation (Murphy, A. J. et al. 2011), within the culture media in which iDiscoids were grown by day 5 (FIG. 2A). Immunofluorescence analysis of iGATA6 cells reveals spindle-shaped clusters of CD34⁺/ERG⁺ endothelial cells, confirming the morphological adaptation (FIG. 2B, FIG. 9A). Critically, these cells were also positive for TAL1 (scl), a key regulator of hemogenic endothelial differentiation and hematopoietic development, and RUNX1, a master transcription factor necessary for hematopoietic stem cell emergence from endothelial cells (FIG. 2C) (Vagapova, E. R. et al., 2018; Yzaguirre, A. D. et al., 2017). These spindle-shaped cells were consistently localized underneath an iGATA6 layer (FIG. 2B), a morphology that is distinctly similar to the in vivo localization of human yolk sac mesoderm, where this tissue is positioned against a basal lamina in the area underneath the yolk sac endoderm (Knoth, M. & Larsen, J. F. 1972) (FIG. 2E). CD34⁺/TAL1⁺ cells were also EGFP⁺, indicating they develop from an iGATA6-derived parental population (FIG. 2B). Flow cytometry analysis of an erythroid marker, CD71, showed a higher level among high EGFP expressing cells (also CD34−) (FIG. 2D). This corroborates the presence of the observed erythroid signature in cluster 5.2 (FIG. 8D). Colony-forming unit (CFU) assays on intact iDiscoids and on iDiscoids enriched for CD34 expressing cells generated CD45⁺ cells (FIGS. 9B, 9C). Enrichment for myeloid-like cells (CD11b⁺) was observed in the CD34-enriched population while erythroid-like cells (CD71⁺, CD235a ⁻ (early stage) or CD235a⁺ (late stage)) were more abundant in whole-tissue populations (FIGS. 9B, 9C). Collectively, these results show the presence of two forms of pro-hematopoietic cells in iDiscoids: one occurring within the GFP-high cells with an erythroid differentiation bias and without transition through a CD34⁺ endothelial cell state and one occurring from CD34⁺ endothelial cells with higher myeloid differentiation potential. These data establish the relevance of this model to probe early hematopoiesis in humans.

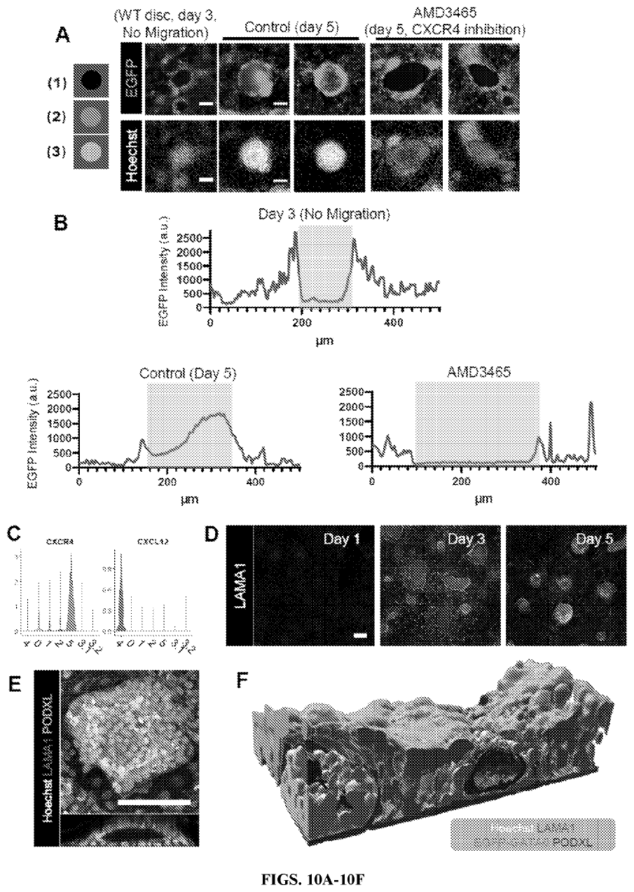

Interaction between embryonic and extra-embryonic cellular progeny leads to formation of amniotic-like cavity with bilaminar discs. Following embryo implantation at around E7 in human, a cavity will develop within the embryonic epiblast, followed by the formation of the bilaminar disc (Shahbazi, M. N. et al. 2016). Beginning on day 3 of iDiscoid development in vitro, conversion of the WT disc structure from a unilayer disc to a three-dimensional cell cluster was observed (WT cluster, FIG. 3A). Over the course of 5 days, iGATA6 cells migrate over the upper surface of these WT clusters, resulting in a contiguous membrane of extraembryonic endoderm-like cells (FIGS. 3A-3D, FIG. 10A). It was found that CXCR4/SDF-1 signaling is critical for this migration; and inhibition of this chemokine pathway via dosage with the CXCR4 antagonist AMD3465 substantially inhibits upward migration and contiguous iGATA6 membrane formation (FIGS. 10A-10B). scRNAseq also revealed that CXCR4 is exclusively expressed by iGATA6 cells (cluster 5), and SDF-1 is expressed by WT cells (FIG. 10C), supporting the functional observations. CXCR4 is expressed in the hypoblast of the chick embryo and is present in anterior human endoderm (Tyser, R. C. V. et al. 2020; Yusuf, F. 2015). Hence, these results indicate that reciprocal CXCR4-SDF1 signaling can have a role in maintenance and stability of the human bilaminar disc.

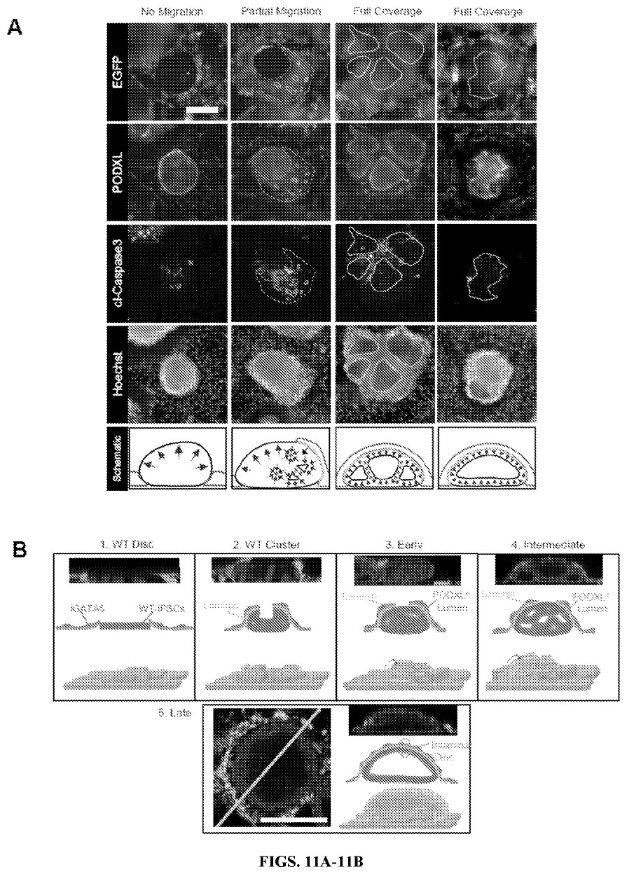

The process of upward iGATA6 migration moves forward in parallel with the deposition of a laminin membrane surrounding the WT clusters. In WT clusters with full iGATA6 coverage, a complete laminin envelope surrounding the WT cluster is observed (FIGS. 10D-10F), mimicking the laminin deposition by primitive endoderm cells following segregation from the epiblast in mouse embryos at E4.5 (Artus, J.2011). The laminin deposition by the iGATA6 cells can subsequently trigger cell polarization (Li, S. et al., 2003; Bedzhov, I. & Zernicka-Goetz, 2014) within cells of the WT cluster, which promotes the initiation of lumen formation. Immunofluorescence analyses was performed for PODXL, an apically-expressed antiadhesive surface protein implicated in human proamniotic lumen and murine proamniotic cavity formation, as well as ZO-1, an apical tight junction protein (FIG. 3B) (Simunovic, M. et al. 2019; Kim, Y. S. et al. 2021; Shahbazi, M. N. et al. 2017; Sozen, B. et al. 2021; Wang, S. et al. 2021; Phua, D. C. et al. 2014). Following upward migration of iGATA6 at days 3 through 5, cavity formation appears to initiate with the formation of local microlumens at the center of PODXL⁺, ZO-1⁺ rosette-like cellular structures (FIGS. 3A-3C). It was observed that within WT clusters for which an iGATA6 layer was not surrounding the cluster, no PODXL⁺ lumen formation was observed (FIG. 3C). The number of clusters with no coverage progressively decreases by day 5 as the migration of iGATA6 cells moves forward in most clusters (FIG. 3D). Cleaved Caspase 3 concentrated within areas near lumens was also detected, coinciding with areas of iGATA6 coverage of WT clusters (FIG. 11A). Apoptosis is most pronounced in intermediate stage lumens, with high levels of apoptosis within the inner membranes separating multiple lumens, indicaitng cell death plays a role in iDiscoid lumen formation and expansion (FIG. 11A). Ultimately, the two cell monolayers of embryonic and extraembryonic fates are positioned on either side of a laminin membrane and polarized in opposing directions (FIG. 11B; late stage), forming a cellular arrangement similar to the bilaminar disc in the post-implantation E12 human embryo (O'Rahilly, R. et al., 1987).

Taken together, this study shows morphogenetic events that collectively result in a bilaminar disc-like structure within the iDiscoid system, initiating with ECM deposition by iGATA6 cells migrating laterally over the WT cluster and proceeding via cell detachment from the apical surfaces of polarized cells and apoptosis of cells outside of the disc (FIG. 11A).

Specification of an amnion-like domain in iDiscoids. Primate amniogenesis is structurally and temporally distinct from mouse, highlighting the need for human model systems to provide a window into understanding human-specific developmental dynamics (Yang, R. et al. 2021). Following lumenogenesis within the epiblast of the post-implantation embryo, the cells of the epiblast undergo dorsal-ventral patterning to separate the columnar epiblast from squamous amniotic ectoderm (Zheng, Y. et al. 2019; Hertig, A. T. & Rock, J. 1949). Similarly, the spontaneous appearance of a domain within the WT discs expressing the amnion co-markers ISL1 and AP-2a was observed, while expression of NANOG is lacking (FIG. 3E-3G, FIG. 12A). This tissue spatially segregates within the cavitated sac, with the amnion-like tissue positioning away from the bilaminar iGATA6/WT disc, against the tissue culture dish. WT cells within the bilaminar iGATA6/WT disc display expression of NANOG and exhibit a columnar morphology (FIGS. 3E-3G, FIG. 12B).

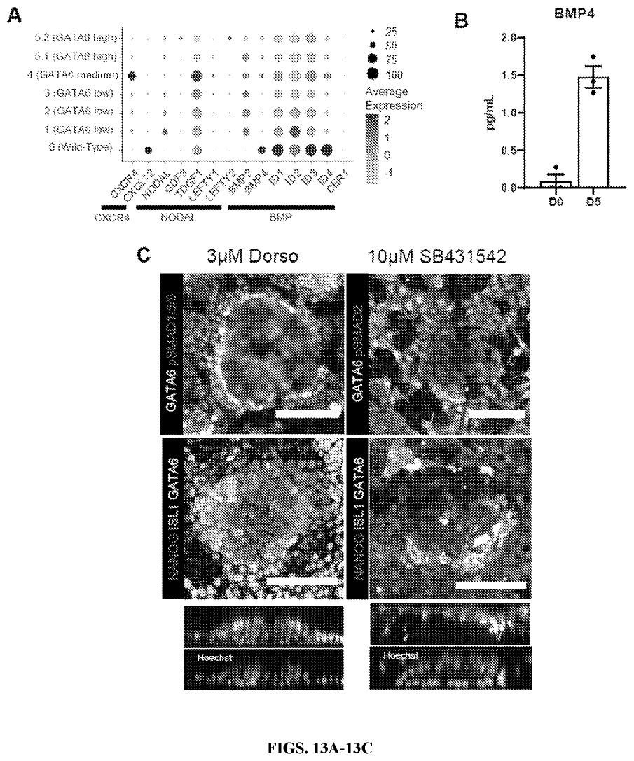

Recently, the active role of primate amniotic tissue to promote BMP4 signaling has been shown, acting downstream of ISL1 (Yang, R. et al. 2021). It was also reported that BMP4 can promote the formation of ISL1V amniotic tissue in a human embryo model (Zheng, Y. et al. 2019). The investigation of BMP4 secretion into the culture media demonstrated a strong increase by day 5, which correlated with the detection of BMP4 targets at day 5 by scRNAseq (FIGS. 13A-13B). Immunofluorescence staining for the BMP4 effectors pSMAD1, pSMAD5, and pSMAD8 revealed BMP4 signal transduction in a ring pattern concentrated at the edges of the iDiscoid (FIG. 3H). This pattern was not fully aligned with the layer of amnion gene expression, indicating BMP4-mediated signaling may not be active in all parts of the amnion-like population within the iDiscoid system.

Immunofluorescent staining for pSMAD2, a surrogate for active NODAL signaling, shows a higher NODAL response in the cells positioned in the amnion-like layer (FIG. 3H). Investigation of the day 5 scRNAseq dataset reveals expression of NODAL transcripts within the iGATA6 compartment and the NODAL co-receptor TDGF1 (Cripto-1) within the WT cluster (FIG. 13A). Inhibition of BMP signaling using a small molecule inhibitor, dorsomorphin, showed effective suppression of SMAD1/5/8 phosphorylation (FIG. 13C), but did not fully prevent the specification of ISL1-expressing amnion-like tissue (FIG. 3I, FIG. 13C). However, NODAL inhibition via SB431562 led to substantial elimination of ISL1-expressing cells within iDiscoids (FIG. 3I, FIG. 13C). Notably, the expression of NANOG within the WT clusters was also substantially decreased, while cavity formation was preserved (FIG. 13C, Hoechst vertical slice). These data indicate an essential role for yolk sac-derived NODAL signaling both for maintenance of pluripotent cells, and as an alternate or complementary driver of the human amnion specification. This finding is also in line with recent studies on NODAL-mediated effects in PSCs (Chhabra, S. et al., 2019; Guo, G. et al. 2021).

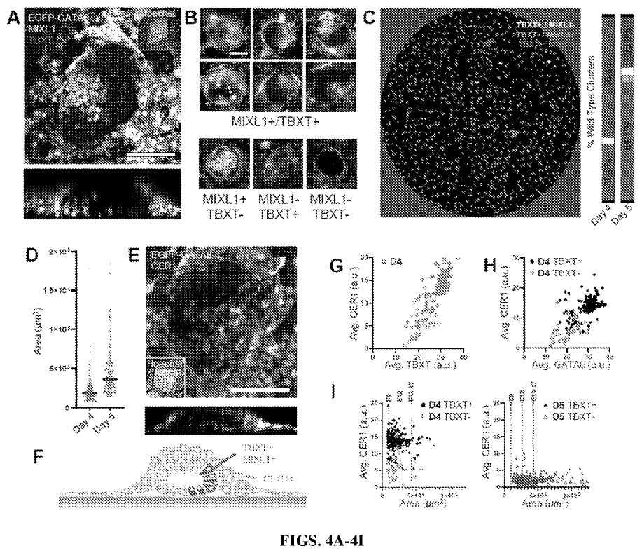

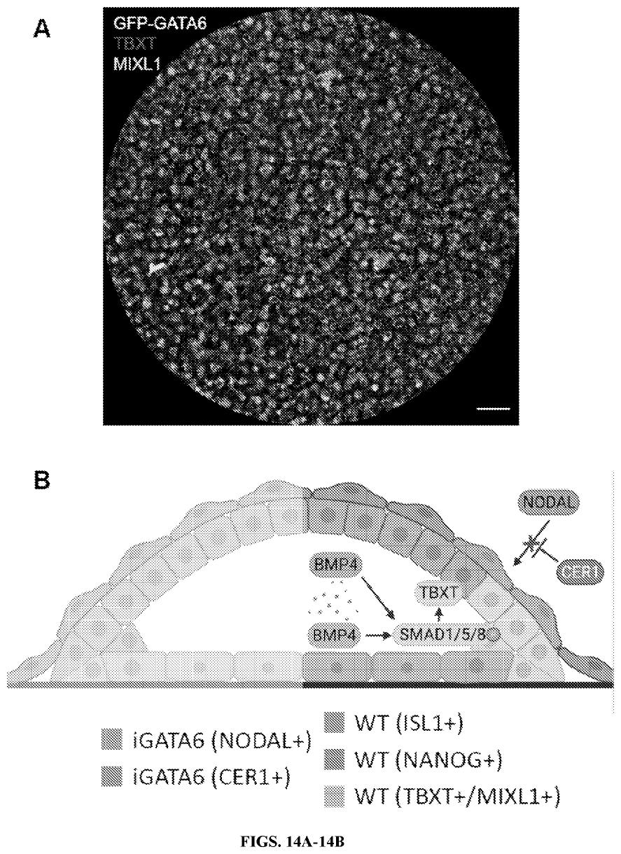

Primitive streak-like domains arise in iDiscoids. The primitive streak, marked by TBXT (Brachyury), establishes the onset of gastrulation and is formed in the posterior regions of the embryo (Mikawa, T. et al., 2004). This study reveals the emergence of distinct domains expressing TBXT and MIXL1 in ˜65% of WT clusters at day 4 in iDiscoids (FIGS. 4A-4C, FIG. 14A). These domains are distinct from iGATA6-derived TBXT-expressing cells (high GATA6 expressing population 5.2) (FIGS. 1D, 1E, FIGS. 7D, 7E), lacking EGFP expression and exhibiting asymmetric patterns (FIGS. 4A, 4B, FIG. 14A). This observation indicates the symmetry breaking and the emergence of a primitive streak-like area within iDiscoid structures mimicking the onset of gastrulation in post-implantation epiblast in native human embryos. Notably, domains that were TBXT/MIXL1 positive had a distinctive polar expression within the WT clusters, while clusters that were positive for only a single marker lacked polar compartmentalization (FIG. 4B). These domains specified transiently, with the proportion of observed streak-like cell clusters decreasing by day 5 (FIG. 4C). This decrease is accompanied by an increase in average cluster size between day 4 and 5 (FIG. 4D).

The next experiment sought to understand important parameters for the development of TBXT-positive poles in WT cells. In non-human primate embryos at E11, CER1 has been shown to be expressed throughout the visceral endoderm; over the course of several hours, expression of CER1 becomes confined to the areas of the visceral endoderm near the epiblast (Sasaki, K. et al. 2016). In mouse, anterior positioning of Cer1 defines and positions the posterior domain, enabling the proper initiation of the primitive streak formation (Rodriguez, T. A. et al., 2005). In iDiscoids, domains of CER1 expression was identified in iGATA6-derived extraembryonic cells surrounding the WT clusters (FIGS. 4E, 4F). Comparison of CER1 and TBXT expression intensities within iDiscoids reveals a sharp positive correlation between average radial CER1 expression and TBXT⁺ domain specification (FIG. 4G). Slightly lower levels of CER1 expression result in a substantially lower level of TBXT expression within the associated WT cluster (FIG. 4G). Investigation of induced GATA6 (EGFP) expression intensity reveals that CER1 is expressed at a higher level around WT clusters with higher average local GATA6 induction, and these clusters have a higher rate of TBXT positivity (FIG. 4H). This strong correlation of streak-domain specification was independent of WT disc area (FIG. 4I). These data highlight a central role for CER1 involvement, but not area, in streak-like domain specification. These cells appeared to overlie the TBXT-expressing streak-like poles, rather than opposing the location of these poles. Currently, data for CER1 positioning in human and primate embryos is limited (Mole, M. A. et al. 2021). However, murine and human Cerberus orthologs show divergence in structure, and investigation of the binding affinities of human CER1 show a high affinity for NODAL, but a minimal affinity for BMP2 and BMP4 (Katoh, M. & Katoh, M. 2006; Aykul, S. et al., 2015). In iDiscoids, CER1-mediated spatial inhibition of NODAL signaling can prime WT cells for activation of TBXT and MIXL1 by BMP4 and mediate downregulation of NANOG at the base edge of the cavity (FIG. 14B) (Zhang, P. et al. 2008). These data indicate that the self-organization of iGATA6 cell population, which positions high GATA6 expressing cells around WT clusters, contributes to the subsequent specification of a TBXT⁺/MIXL1⁺ streak-like domain via patterning by a CER1⁺ iGATA6 pole. iDiscoids, presented here, develop both embryonic and extraembryonic lineages with several features observed together for the first time in an in vitro human developmental model.

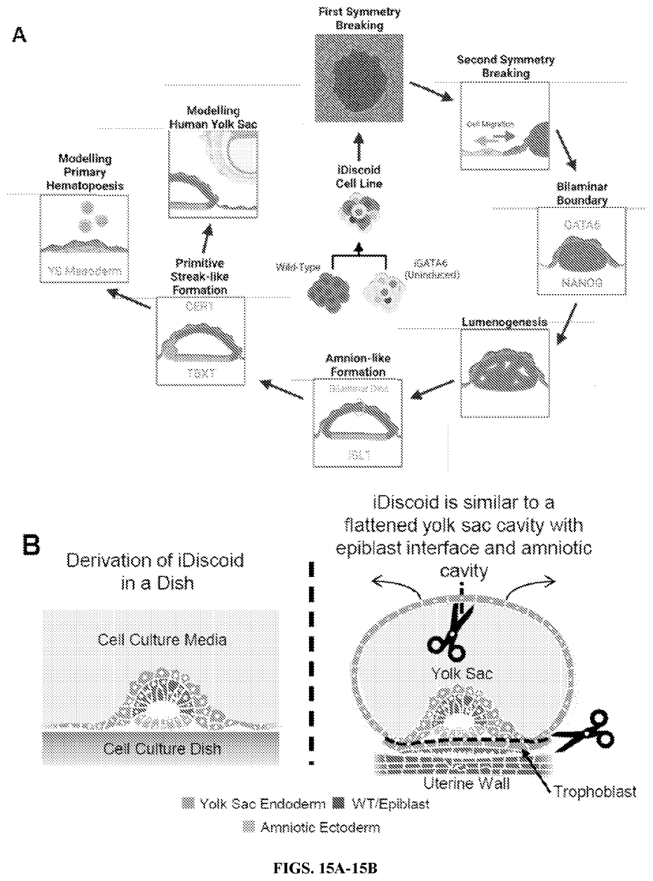

These features show close proximity to native post-implantation human embryogenesis yet are uniquely observed in an hiPSC-based system (FIG. 15). The iDiscoid platform highlights the power of niche engineering and tissue co-differentiation to promote morphogenetic events in vitro. It demonstrates that a combination of genetic circuit-based heterotypic tissue engineering with the formation of tissue-tissue boundaries in 2D enables geometric confinement and subsequent self-organization in 3D (i.e., lumenogenesis).

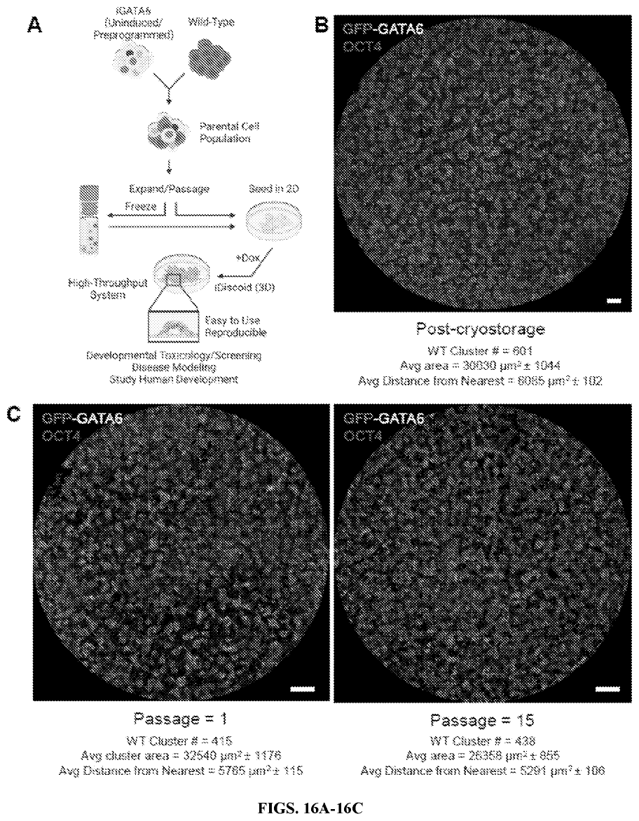

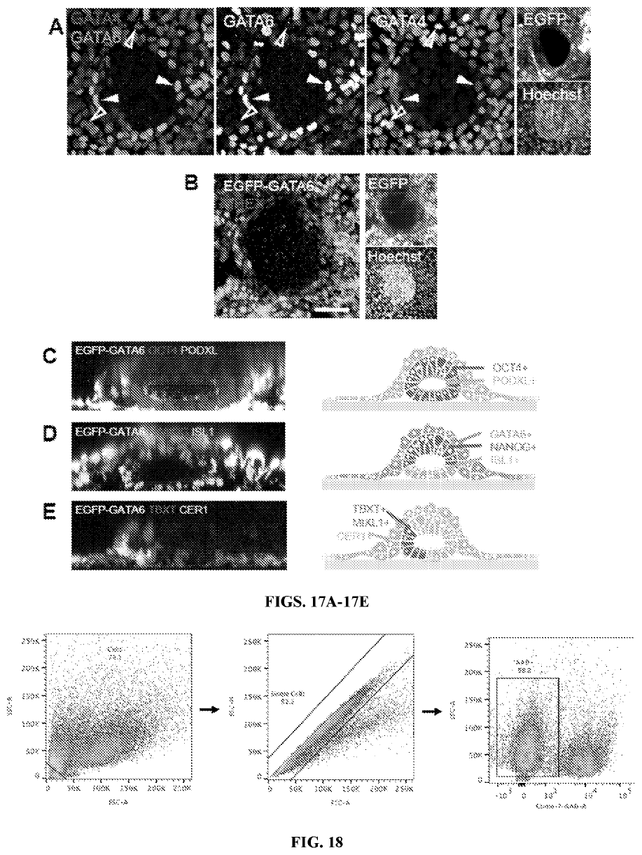

The extraembryonic tissues of iDiscoids are genetically pre-programmed into undifferentiated hiPSCs with an inducible genetic switch and mixed with WT hiPSCs. The undifferentiated cell mix can be expanded, cryostored, and shipped for use on demand using only conventional 2D culture plates and addition of a single small molecule, Dox (FIG. 16). The high-throughput format, efficient generation, scalability, compatibility with live imaging, and an easy-to-implement protocol enables consistent establishment across different labs, facilitating research in this area. These characteristics and the potential to be produced from hiPSCs with diverse genetic backgrounds (FIG. 17), enable studies that uncover principles that control developmental fates and forms in humans, opening potential new routes for high throughput drug testing, developmental toxicology, and tractable disease modelling with fewer ethical concerns.

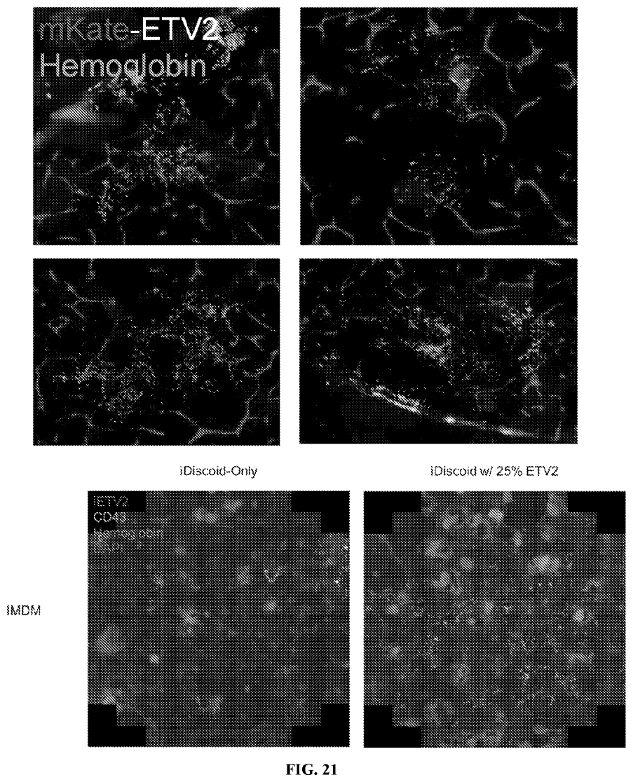

Clonal supplementation for tissue by design features: Specific pre-programmed organ or hematopoietic progenitor lineages can be clonally isolated prior to system induction and supplemented at higher levels into the system to increase the amount of those lineages that develop, enabling custom by-design tissues pre-programmed into the invention (FIGS. 20A-20B).

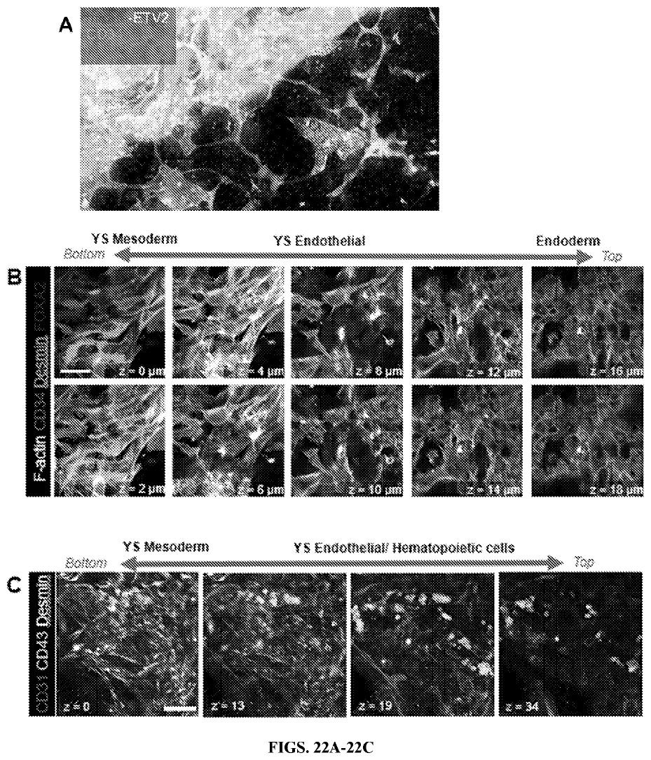

Self-vascularizing inducible yolk sac features: Cells engineered to inducibly express ETV2 can be integrated into the cell culture system to enhance vascularization and endothelial cell formation within tissues, contributing to the formation of a self-vascularizing yolk sac model system for use in understanding yolk sac capabilities and for the development of personalized therapeutics (FIG. 21). ETV2 is expressed in neighboring cells in contact with the GATA6-expressing cell. In this case, ETV2 genetic circuit is engineered in an independent cell type and is controlled by doxycycline. The two cell lines are mixed to form the final organoid in some cases.

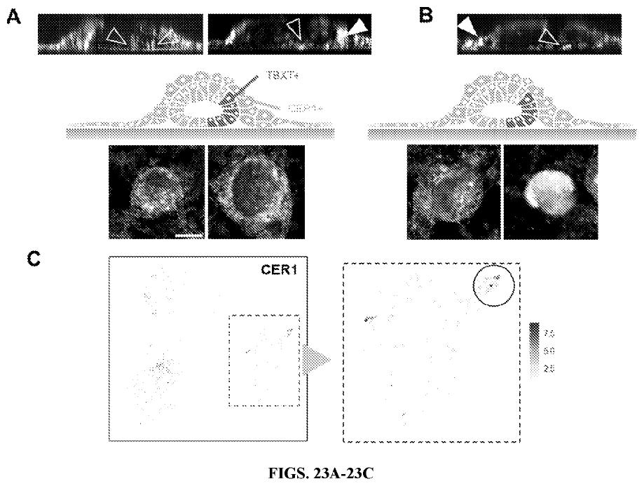

Engineered formation of advanced blood islands and hematopoietic lineages from the human yolk sac: Cells engineered to inducibly express ETV2 can be combined with cells engineered to inducibly express GATA6, and in combination they develop enhanced yolk sac features, including cell identity equivalent to the in vivo human yolk sac, enhanced yolk sac-like vascularization, and the formation of yolk sac-derived hematopoietic cells in physiologically-relevant organized blood island structures (FIGS. 22A-22C).

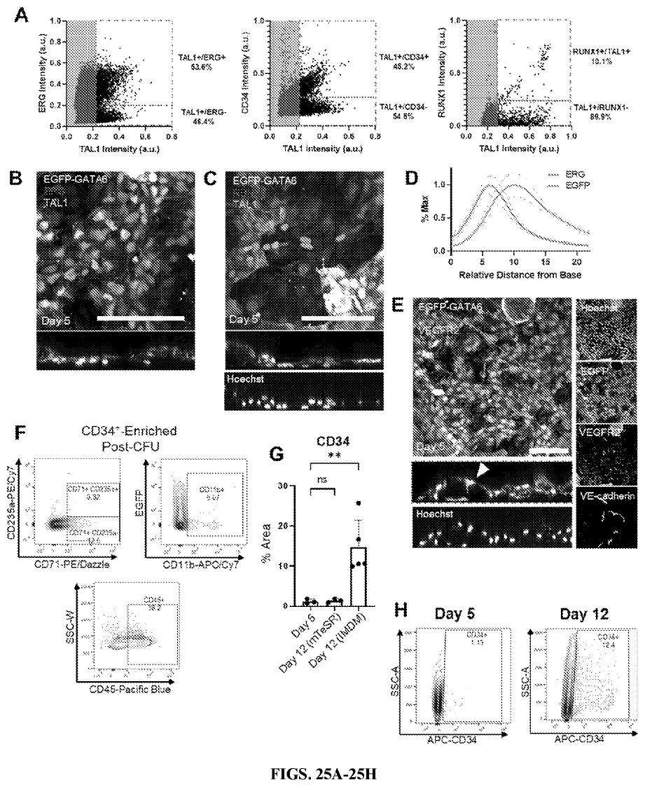

Anteriorizing activity from anterior hypoblast-like tissue leading to development of neural lineages: This system can produce ectodermal outgrowths with neural features, including expression of markers related to neural plate and surface ectoderm, due to anteriorizing signaling produced by the GATA6 cells, including the proteins CER1 and LEFTY1. These neural features will develop into organized structures that can be harvested for analysis or cultured independently to enhance their features (FIGS. 24A-24C).

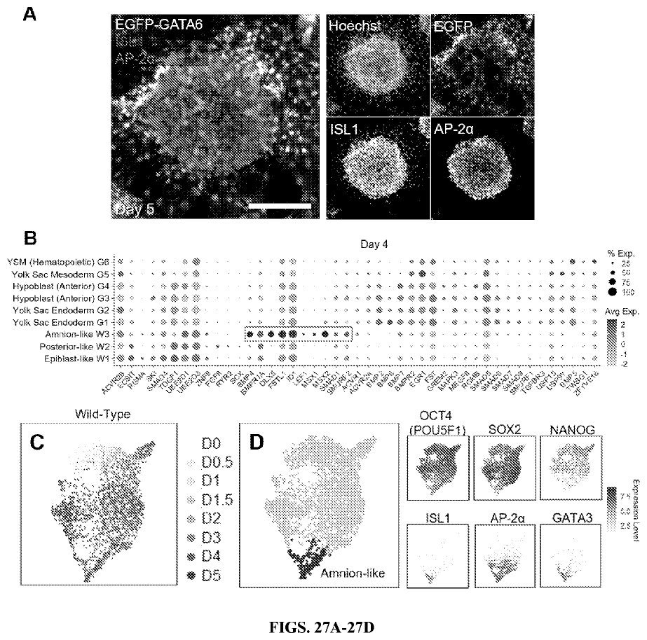

Maturation of hematopoietic lineages over extended periods of maintenance within the system: This system can be sustained in culture for long periods of time, and when given long periods to develop to show development of hematopoietic progenitor lineages including hematopoietic stem cells, erythroid progenitors, and myeloid progenitors. By day 23, these lineages can have gene regulatory networks that are significantly similar to in vivo human embryonic hematopoietic lineages (FIGS. 27A-27D).

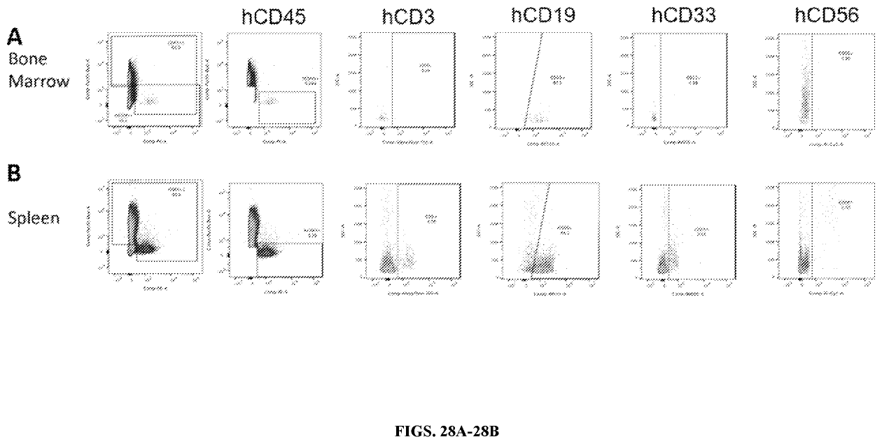

In vivo functional reconstitution of the blood system: This technology can recolonize and reconstitute the bone marrow and spleen with hematopoietic cells in vivo, including B cells, T cells, NK cells, myeloid cells, and erythroid cells, enabling it to be used for autogenic transplantation of healthy tissue following injury or irradiation (FIGS. 28A-28B).

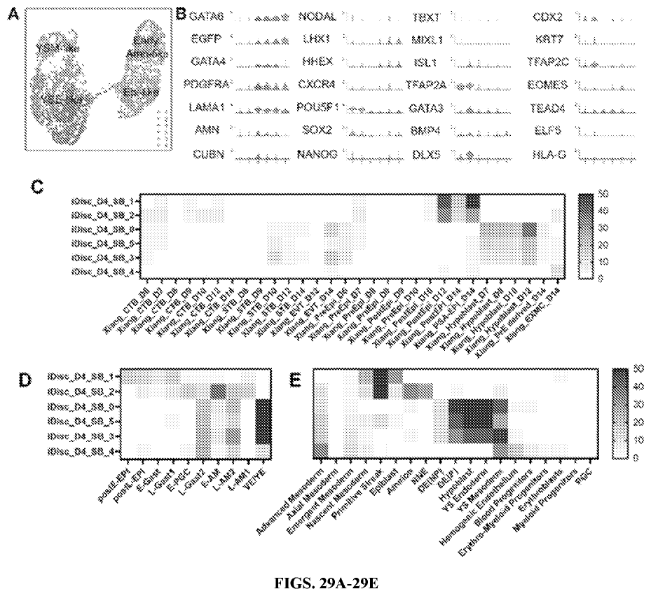

Use of small molecules and proteins in the media to impact system state: This invention can be altered via introduction of chemicals and exogenous proteins to the cell culture media, altering the fates that are acquired by the cells in the system. The NODAL inhibitor SB431542 can be added to reprogram system to acquire and maintain a state similar to early human and primate amnion prior to acquiring late amnion features. This can also be used to induce the system to acquire markers of human trophoblast, enabling the system to be used for development of model placental tissue (FIGS. 29A-29E).

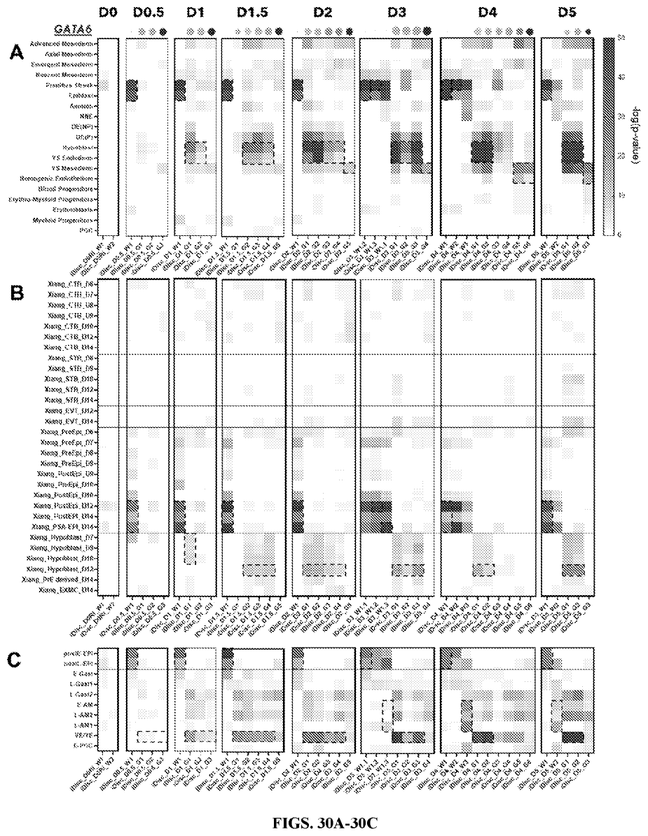

Reestablishing embryonic identity in un-engineered cell types exposed to the system: The specific combination of factors secreted by the system can impact the gene regulatory network of cells exposed to the media of the culture, reprogramming them into a younger, more embryo-like state. This alleviates age-related features from the cells that are associated with disease and degeneration of tissues, enabling therapeutic impact in the context of age reversal and tissue regeneration (FIGS. 30A-30C).

### Example 3

This study describes a new in vitro induced pluripotent cell-based tool that can be used for research in the post-inner cell mass stages of the human embryo. The design principles governing human embryogenesis remain a mystery due to limited accessibility to post-implantation human embryo for both technical and ethical reasons. Here, the study presents an in vitro stem cell-based platform termed iDiscoids. iDiscoids are produced from human induced pluripotent stem cells and are a unique model for human embryo study because they exhibit the development following features together with self-timing and organization: reciprocal co-development of embryonic and extra-embryonic tissues; segregation of epiblast and hypoblast; formation of human bilaminar disc-like structures; lumenogenesis; development of the amniotic cavity; development of the posterior axis with primitive streak features; and development yolk sac with hematopoietic characteristics, including erythrogenesis and myelogenesis; and reprogramming of induced pluripotent stem cells from a primed pluripotent state to a naïve pluripotent state. These events are achieved using an isogenic cell population containing wild type cells and a population of genetically engineered cells to express heterogenous levels of the human transcription factor GATA6 that are seeded and induced in a 2D culture. Upon seeding and activation of the engineered gene circuit, the study shows formation of extra-embryonic endoderm lineages, self-organization, cell migration and stepwise boundary establishment that collectively coax late blastocyst-to-gastrula-like morphogenetic events in wild-type hiPSCs.

Current human embryo models focus on the development of a limited number of embryo features, and exclude the primitive endoderm and yolk sac from study. Existing models individually develop a subset of embryo-relevant features such as amnion, lumenogenesis, and posterior axis, but iDiscoids are unique because they develop all features listed together in a single self-organized and self-timed system. Additionally, no model has demonstrated the emergence of hematopoietic tissues within an organized embryo-like system, nor have they show de novo reprogramming of iPSCs towards a naïve state. The self-organization and self-specification of these embryo-like events in sequence is novel, and only parts of the overall process demonstrated in iDiscoids are able to develop in other models.

Robust, scalable and easy-to-use models of human embryogenesis can enable real-time interrogation of development across different labs. Subsequently, this model is used to study yolk sac hematopoiesis as well as signaling events governing cavitation and amnion fate acquisition in hiPSCs. Collectively, iDiscoids offer an easy-to-use, high-throughput, scalable platform to probe multifaceted aspects of post-implantation development in a human-based system using a genetically pre-programmed niche. This system can be used for understanding of human developmental steps beyond implantation, where real embryos become hidden within the uterine wall and are unobservable foe legal and ethical reasons. This model can be used for precision medicine, to explore likely embryo defects involving the yolk sac, early hematopoiesis, and amnion, and for the exploration and development of treatments on an individualized basis. This model can also be used to improve the efficacy of in vitro fertilization through investigation of characteristics of the yolk sac and epiblast that lead to the most successful emergence of important embryonic features.

List any novel or unusual features of the invention.
This system develops hematopoietic cells and tissues critical for population of a blood system, meaning this technology can be served as a source of donor cells for patients requiring blood system regeneration following disease.
This system robustly develops tissue similar to the primitive endoderm, a critical tissue for the success of IVF pregnancies. Supplementation of low quality embryos with tissue developed via this technology can substantially increase the number of high quality embryos available to women seeking IVF.
This system shows aspects of naïve reprogramming of pluripotent tissue, which has applications for regenerative therapy and treatment of age-related diseases.
This system can serve as a model for embryogenesis for the purposes of testing drugs and therapies involving pregnancy and prenatal tissues.
This system can be cryostored for long periods and thawed on-demand for use.

This system is reliant only on induction of a gene circuit, and can be cultured, stored, shipped, and activated on demand. This system is cultured in 2D on a cell culture dish and remains attached as it acquires 3D features in a bilaminar disc area, resulting in a morphology similar to an “unrolled” embryo. This system develops many bilaminar disc structures in a single cell culture well, and enabling high-throughput analysis of a hundreds of human disc features in a single experiment. Along with the development of these bilaminar disc structures, what is observed includes the development of cells with identities similar to amnion, the primitive and pro-definitive hematopoietic system, and the primitive streak, including the development of rare early-embryo cell types. Additionally, this system shows reprogramming of induced pluripotent stem cells to a naïve state, which can be utilized for human developmental studies and regenerative medicine.

This invention encompasses the use of genetic engineering within a specific cell type and its combination with a second cell type in order to enable unique self-organization and the development of secondary cell types of interest.

The application for this technology is threefold, which synergizes with the reproducibility and ease of use of the method, which enables commercialization and use across different lab environments with limited technical challenges.
The first is as a diagnostic tool for assessing the impact of drugs and toxins on early stages of embryonic development, as a step that can be taken in drug development prior to mammalian in vitro tests. 748 Early Phase 1 or Phase 1 clinical studies for drugs or biologics were posted in 2021 on ClinicalTrials.gov, each of which represents a company that could benefit from the use of this product.
Additionally, this product can be useful for improving the efficacy of IVF. Primitive endoderm quality is one of the most important factors controlling the success of an embryo during IVF, and autogenic supplementation of the primitive endoderm in low-quality embryos has the potential to substantially improve IVF success. According to the CDC, 330,773 women sought assisted reproductive therapy (ART) in the US in 2019, and 29% of these women sought ART due to diminished ovarian reserve, a condition where the number of high-quality embryos produced are significantly decreased. This results in a US market of roughly 96,000 individuals yearly.
Thirdly, this product has the potential to be used for autogenic or allogenic development of hematopoietic cells for hematopoietic stem cell transplants. This product could take the form of a bioreactor-style platform that uses a patient's own cells for expansion of hematopoietic stem cells for regeneration of their blood system. According to the HRSA, 23,000 bone marrow or cord blood transplants were performed in the US in 2019. Additionally, each year, approximately 18,000 people are diagnosed with a life-threatening illness for which a bone marrow or cord blood transplant is the best treatment option, and for which our product could represent an effective treatment.

SEQUENCES
(pENTR_L1_rtTA3-2A-Hygro_L2 (6))
SEQ ID NO: 1
ctttcctgcgttatcccctgattctgtggataaccgtattaccgcctttgagtgagctgataccg
ctcgccgcagccgaacgaccgagcgcagcgagtcagtgagcgaggaagcggaagagcgcccaata
cgcaaaccgcctctccccgcgcgttggccgattcattaatgcagctggcacgacaggtttcccga
ctggaaagcgggcagtgagcgcaacgcaattaatacgcgtaccgctagccaggaagagtttgtag
aaacgcaaaaaggccatccgtcaggatggccttctgcttagtttgatgcctggcagtttatgggg
gcgtcctgcccgccaccctccgggccgttgcttcacaacgttcaaatccgctcccggcggatttg
tcctactcaggagagcgttcaccgacaaacaacagataaaacgaaaggcccagtcttccgactga
gcctttcgttttatttgatgcctggcagttccctactctcgcgttaacgctagcatggatgtttt
cccagtcacgacgttgtaaaacgacggccagtcttaagctcgggccccaaataatgattttattt
tgactgatagtgacctgttcgttgcaacaaattgatgagcaatgcttttttataatgccaacttt
gtacaaaaaagcaggctggatccccgggtaccggtcgccaccatgtctaggctggacaagagcaa
agtcataaacggagctctggaattactcaatggtgtcggtatcgaaggcctgacgacaaggaaac
tcgctcaaaagctgggagttgagcagcctaccctgtactggcacgtgaagaacaagcgggccctg
ctcgatgccctgccaatcgagatgctggacaggcatcatacccacttctgccccctggaaggcga
gtcatggcaagactttctgcggaacaacgccaagtcataccgctgtgctctcctctcacatcgcg
acggggctaaagtgcatctcggcacccgcccaacagagaaacagtacgaaaccctggaaaatcag
ctcgcgttcctgtgtcagcaaggcttctccctggagaacgcactgtacgctctgtccgccgtggg
ccactttacactgggctgcgtattggaggaacaggagcatcaagtagcaaaagaggaaagagaga
cacctaccaccgattctatgcccccacttctgagacaagcaattgagctgttcgaccggcaggga
gccgaacctgccttccttttcggcctggaactaatcatatgtggcctggagaaacagctaaagtg
cgaaagcggcgggccgaccgacgcccttgacgattttgacttagacatgctcccagccgatgccc
ttgacgattttgaccttgacatgctccccggggaattggagggccgcggcagcctgctgacctgc
ggcgacgtggaggaaaaccccggccccatgaaaaagcctgaactcaccgcgacgtctgtcgagaa
gtttctgatcgaaaagttcgacagcgtctccgacctgatgcagctctcggagggcgaagaatctc
gtgctttcagcttcgatgtaggagggcgtggatatgtcctgcgggtaaatagctgcgccgatggt
ttctacaaagatcgttatgtttatcggcactttgcatcggccgcgctcccgattccggaagtgct
tgacattggggaattcagcgagagcctgacctattgcatctcccgccgtgcacagggtgtcacgt
tgcaagacctgcctgaaaccgaactgcccgctgttctgcagccggtcgcggaggccatggatgcg
atcgctgcggccgatcttagccagacgagcgggttcggcccattcggaccgcaaggaatcggtca
atacactacatggcgtgatttcatatgcgcgattgctgatccccatgtgtatcactcctggcaaa
ctgtgatggacgacaccgtcagtgcgtccgtcgcgcaggctctcgatgagctgatgctttgggcc
gaggactgccccgaagtccggcacctcgtgcacgcggatttcggctccaacaatgtcctgacgga
caatggccgcataacagcggtcattgactggagcgaggcgatgttcggggattcccaatacgagg
tcgccaacatcttcttctggaggccgtggttggcttgtatggagcagcagacgcgctacttcgag
cggaggcatccggagcttgcaggatcgccgcggctccgggcgtatatgctccgcattggtcttga
ccaactctatcagagcttggttgacggcaatttcgatgatgcagcttgggcgcagggtcgatgcg
acgcaatcgtccgatccggagccgggactgtcgggcgtacacaaatcgcccgcagaagcgcggcc
gtctggaccgatggctgtgtagaagtactcgccgatagtggaaaccgacgccccagcactcgtcc
ggatcgggagatgggggaggctaactgagcggccgcgaattctaccacccagctttcttgtacaa
agttggcattataagaaagcattgcttatcaatttgttgcaacgaacaggtcactatcagtcaaa
ataaaatcattatttgccatccagctgatatcccctatagtgagtcgtattacatggtcatagct
gtttcctggcagctctggcccgtgtctcaaaatctctgatgttacattgcacaagataaaataat
atcatcatgaacaataaaactgtctgcttacataaacagtaatacaaggggtgttatgagccata
ttcaacgggaaacgtcgaggccgcgattaaattccaacatggatgctgatttatatgggtataaa
tgggctcgcgataatgtcgggcaatcaggtgcgacaatctatcgcttgtatgggaagcccgatgc
gccagagttgtttctgaaacatggcaaaggtagcgttgccaatgatgttacagatgagatggtca
gactaaactggctgacggaatttatgcctcttccgaccatcaagcattttatccgtactcctgat
gatgcatggttactcaccactgcgatccccggaaaaacagcattccaggtattagaagaatatcc
tgattcaggtgaaaatattgttgatgcgctggcagtgttcctgcgccggttgcattcgattcctg
tttgtaattgtccttttaacagcgatcgcgtatttcgtctcgctcaggcgcaatcacgaatgaat
aacggtttggttgatgcgagtgattttgatgacgagcgtaatggctggcctgttgaacaagtctg
gaaagaaatgcataaacttttgccattctcaccggattcagtcgtcactcatggtgatttctcac
ttgataaccttatttttgacgaggggaaattaataggttgtattgatgttggacgagtcggaatc
gcagaccgataccaggatcttgccatcctatggaactgcctcggtgagttttctccttcattaca
gaaacggctttttcaaaaatatggtattgataatcctgatatgaataaattgcagtttcatttga
tgctcgatgagtttttctaatcagaattggttaattggttgtaacactggcagagcattacgctg
acttgacgggacggcgcaagctcatgaccaaaatcccttaacgtgagttacgcgtcgttccactg
agcgtcagaccccgtagaaaagatcaaaggatcttcttgagatcctttttttctgcgcgtaatct
gctgcttgcaaacaaaaaaaccaccgctaccagcggtggtttgtttgccggatcaagagctacca
actctttttccgaaggtaactggcttcagcagagcgcagataccaaatactgttcttctagtgta
gccgtagttaggccaccacttcaagaactctgtagcaccgcctacatacctcgctctgctaatcc
tgttaccagtggctgctgccagtggcgataagtcgtgtcttaccgggttggactcaagacgatag
ttaccggataaggcgcagcggtcgggctgaacggggggttcgtgcacacagcccagcttggagcg
aacgacctacaccgaactgagatacctacagcgtgagctatgagaaagcgccacgcttcccgaag
ggagaaaggcggacaggtatccggtaagcggcagggtcggaacaggagagcgcacgagggagctt
ccagggggaaacgcctggtatctttatagtcctgtcgggtttcgccacctctgacttgagcgtcg
atttttgtgatgctcgtcaggggggcggagcctatggaaaaacgccagcaacgcggcctttttac
ggttcctggccttttgctggccttttgctcacatgtt
CDS of rtTA3: 694-1398 of SEQ ID NO: 1
CDS of 2A: 1405-1458 of SEQ ID NO: 1
CDS of Hygromycin: 1459-2499 of SEQ ID NO: 1
CDS of Kanamycin Resistance: 2787-3596 of SEQ ID NO: 1
(JVLR3_PB-TAG-ERP2_with_GATA6-2A-EGFP gateway (4))
SEQ ID NO: 2
ggaaattgtaaacgttaatattttgttaaaattcgcgttaaatttttgttaaatcagctcatttt
ttaaccaataggccgaaatcggcaaaatcccttataaatcaaaagaatagaccgagatagggttg
agtgttgttccagtttggaacaagagtccactattaaagaacgtggactccaacgtcaaagggcg
aaaaaccgtctatcagggcgatggcccactacgtgaaccatcaccctaatcaagttttttggggt
cgaggtgccgtaaagcactaaatcggaaccctaaagggagcccccgatttagagcttgacgggga
aagccggcgaacgtggcgagaaaggaagggaagaaagcgaaaggagcgggcgctagggcgctggc
aagtgtagcggtcacgctgcgcgtaaccaccacacccgccgcgcttaatgcgccgctacagggcg
cgtcgcgccattcgccattcaggctgcgcaactgttgggaagggcgatcggtgcgggcctcttcg
ctattacgccagctggcgaaagggggatgtgctgcaaggcgattaagttgggtaacgccagggtt
ttcccagtcacgacgttgtaaaacgacggccagtgaattgtaatacgactcactatagggcgaat
tggagctcggtattcacgacagcaggctgaataataaaaaaattagaaactattatttaacccta
gaaagataatcatattgtgacgtacgttaaagataatcatgcgtaaaattgacgcatgtgtttta
tcggtctgtatatcgaggtttatttattaatttgaatagatattaagttttattatatttacact
tacatactaataataaattcaacaaacaatttatttatgtttatttatttattaaaaaaaaacaa
aaactcaaaatttcttctataaagtaacaaaacttttaaacattctctcttttacaaaaataaac
ttattttgtactttaaaaacagtcatgttgtattataaaataagtaattagcttaacctatacat
aatagaaacaaattatacttattagtcagtcagaaacaactttggcacatatcaatattatgctc
tcgttaattaactcgagggatccaagctggccgcacgtctccctatcagtgatagagaagtcgac
acgtctcgagctccctatcagtgatagagaaggtacgtctagaacgtctccctatcagtgataga
gaagtcgacacgtctcgagctccctatcagtgatagagaaggtacgtctagaacgtctccctatc
agtgatagagaagtcgacacgtctcgagctccctatcagtgatagagaaggtacgtctagaacgt
ctccctatcagtgatagagaagtcgacacgtctcgagctccctatcagtgatagagaaggtaccc
cctatataagcagagctcgtttagtgaaccgtcagatcgcctggagacgccatccacgctgtttt
gacctccatagaagacaccgggaccgatccagcctccgcggtggcggccatcacaagtttgtaca
aaaaagcaggctaggttcgccaccatggccttgactgacggcggctggtgcttgccgaagcgctt
cggggccgcgggtgcggacgccagcgactccagagcctttccagcgcgggagccctccacgccgc
cttcccccatctcttcctcgtcctcctcctgctcccggggggagagcggggccccggcggcgcca
gcaactgcgggacgcctcagctcgacacggaggggggccggacccccggcccgctcgctgctgct
cagttcctacgcttcgcatcccttcggggctccccacggaccttcggcgcctggggtcgcgggcc
ccgggggcaacctgtcgagctgggaggacttgctgctgttcactgacctcgaccaagccgcgacc
gccagcaagctgctgtggtccagccgcggcgccaagctgagccccttcgcacccgagcagccgga
ggagatgtaccagaccctcgccgctctctccagccagggtccggccgcctacgacggcgcgcccg
gcggcttcgtgcactctgcggccgcggcggcagcagccgcggcggcggccagctccccggtctac
gtgcccaccacccgcgtgggttccatgctgcccggcctaccgtaccacctgcaggggtcgggcag
tgggccagccaaccacgcgggggcgcgggcgcgcaccccggctggcctcaggcctcggccgacag
ccctccatacggcagcggaggcggcgcggctggcggcggggccgcggggcctggcggcgctggct
cagccgcggcgcacgtctcggcgcgcttcccctactctcccagcccgcccatggccaacggcgcc
gcgcgggagccgggaggctacgcggcggcgggcagtgggggcgcgggaggcgtgagcggcggcgg
cagtagcctggcggccatgggcggccgcgagccccagtacagctcgctgtcggccgcgcggccgc
tgaacgggacgtaccaccaccaccaccaccaccaccaccaccatccgagcccctactcgccctac
gtgggggcgccactgacgcctgcctggcccgccggacccttcgagaccccggtgctgcacagcct
gcagagccgcgccggagccccgctcccggtgccccggggtcccagtgcagacctgctggaggacc
tgtccgagagccgcgagtgcgtgaactgcggctccatccagacgccgctgtggcggcgggacggc
accggccactacctgtgcaacgcctgcgggctctacagcaagatgaacggcctcagccggcccct
catcaagccgcagaagcgcgtgccttcatcacggcggcttggattgtcctgtgccaactgtcaca
ccacaactaccaccttatggcgcagaaacgccgagggtgaacccgtgtgcaatgcttgtggactc
tacatgaaactccatggggtgcccagaccacttgctatgaaaaaagagggaattcaaaccaggaa
acgaaaacctaagaacataaataaatcaaagacttgctctggtaatagcaataattccattccca
tgactccaacttccacctcttctaactcagatgattgcagcaaaaatacttcccccacaacacaa
cctacagcctcaggggcgggtgccccggtgatgactggtgcgggagagagcaccaatcccgagaa
cagcgagctcaagtattcgggtcaagatgggctctacataggcgtcagtctcgcctcgccggccg
aagtcacgtcctccgtgcgaccggattcctggtgcgccctggccctggccggatctggcgccacc
aacttctctctgctgaagcaggccggcgacgtggaggagaacccaggcccaatggtgagcaaggg
cgaggagctgttcaccggggtggtgcccatcctggtcgagctggacggcgacgtaaacggccaca
agttcagcgtgtccggcgagggcgagggcgatgccacctacggcaagctgaccctgaagttcatc
tgcaccaccggcaagctgcccgtgccctggcccaccctcgtgaccaccctgacctacggcgtgca
gtgcttcagccgctaccccgaccacatgaagcagcacgacttcttcaagtccgccatgcccgaag
gctacgtccaggagcgcaccatcttcttcaaggacgacggcaactacaagacccgcgccgaggtg
aagttcgagggcgacaccctggtgaaccgcatcgagctgaagggcatcgacttcaaggaggacgg
caacatcctggggcacaagctggagtacaactacaacagccacaacgtctatatcatggccgaca
agcagaagaacggcatcaaggtgaacttcaagatccgccacaacatcgaggacggcagcgtgcag
ctcgccgaccactaccagcagaacacccccatcggcgacggccccgtgctgctgcccgacaacca
ctacctgagcacccagtccgccctgagcaaagaccccaacgagaagcgcgatcacatggtcctgc
tggagttcgtgaccgccgccgggatcactctcggcatggacgagctgtacaagtaagcttaccca
gctttcttgtacaaagtggtgatctagctaaaacgcggcctcgaatcgaattccgcccccccccc
tctccctcccccccccctaacgttactggccgaagccgcttggaataaggccggtgtgcgtttgt
ctatatgttattttccaccatattgccgtcttttggcaatgtgagggcccggaaacctggccctg
tcttcttgacgagcattcctaggggtctttcccctctcgccaaaggaatgcaaggtctgttgaat
gtcgtgaaggaagcagttcctctggaagcttcttgaagacaaacaacgtctgtagcgaccctttg
caggcagcggaaccccccacctggcgacaggtgcctctgcggccaaaagccacgtgtataagata
cacctgcaaaggcggcacaaccccagtgccacgttgtgagttggatagttgtggaaagagtcaaa
tggctctcctcaagcgtattcaacaaggggctgaaggatgcccagaaggtaccccattgtatggg
atctgatctggggcctcggtgcacatgctttacatgtgtttagtcgaggttaaaaaacgtctagg
ccccccgaaccacggggacgtggttttcctttgaaaaacacgatgataatatggccacaaccatg
gtgagcaagggcgaggagctgttcaccggggtggtgcccatcctggtcgagctggacggcgacgt
aaacggccacaagttcagcgtgtccggcgagggcgagggcgatgccacctacggcaagctgaccc
tgaagttcatctgcaccaccggcaagctgcccgtgccctggcccaccctcgtgaccaccctgacc
tacggcgtgcagtgcttcagccgctaccccgaccacatgaagcagcacgacttcttcaagtccgc
catgcccgaaggctacgtccaggagcgcaccatcttcttcaaggacgacggcaactacaagaccc
gcgccgaggtgaagttcgagggcgacaccctggtgaaccgcatcgagctgaagggcatcgacttc
aaggaggacggcaacatcctggggcacaagctggagtacaactacaacagccacaacgtctatat
catggccgacaagcagaagaacggcatcaaggtgaacttcaagatccgccacaacatcgaggacg
gcagcgtgcagctcgccgaccactaccagcagaacacccccatcggcgacggccccgtgctgctg
cccgacaaccactacctgagcacccagtccgccctgagcaaagaccccaacgagaagcgcgatca
catggtcctgctggagttcgtgaccgccgccgggatcactctcggcatggacgagctgtacaagt
aaagcggccgcgatctttttccctctgccaaaaattatggggacatcatgaagccccttgagcat
ctgacttctggctaataaaggaaatttattttcattgcaatagtgtgttggaattttttgtgtct
ctcactcggaaggacatatgggagggcaaatcatttaaaacatcagaatgagtatttggtttaga
gtttggcaacatatgccatatgctggctgccatgaacaaaggtggctataaagaggtcatcagta
tatgaaacagccccctgctgtccattccttattccatagaaaagccttgacttgaggttagattt
tttttatattttgttttgtgttatttttttctttaacatccctaaaattttccttacatgtttta
ctagccagatttttcctcctctcctgactactcccagtcatagctgtccctcttctcttatgaag
atccctcgacctgcaggaattcgattacttaagcctaggatccagatctgtacattcgaatcgat
gaattcgttaaccatggaagcttctgcagcggccgcactagcggtggcggcctcgacattgatta
ttgactagttattaatagtaatcaattacggggtcattagttcatagcccatatatggagttccg
cgttacataacttacggtaaatggcccgcctggctgaccgcccaacgacccccgcccattgacgt
caataatgacgtatgttcccatagtaacgccaatagggactttccattgacgtcaatgggtggag
tatttacggtaaactgcccacttggcagtacatcaagtgtatcatatgccaagtacgccccctat
tgacgtcaatgacggtaaatggcccgcctggcattatgcccagtacatgaccttatgggactttc
ctacttggcagtacatctacgtattagtcatcgctattaccatgatgatgcggttttggcagtac
atcaatgggcgtggatagcggtttgactcacggggatttccaagtctccaccccattgacgtcaa
tgggagtttgttttgactagtggagccgagagtaattcatacaaaaggagggatcgccttcgcaa
ggggagagcccagggaccgtccctaaattctcacagacccaaatccctgtagccgccccacgaca
gcgcgaggagcatgcgcccagggctgagcgcgggtagatcagagcacacaagctcacagtccccg
gcggtggggggaggggcgcgctgagcgggggccagggagctggcgcggggcaaactgggaaagtg
gtgtcgtgtgctggctccgccctcttcccgaggggggggagaacggtatataagtgcggtagtcg
ccttggacgttctttttcgcaacgggtttgccgtcagaacgcaggtgagtggcgggtgtggcttc
cgcgggccccggagctggagccctgctctgagcgggccgggctgatatgcgagtgtcgtccgcag
ggtttagctgtgagcattcccacttcgagtgggggggggggggtgagagtgcgaggcctagcggc
aaccccgtagcctcgcctcgtgtccggcttgaggcctagcgtggtgtccgccgccgcgtgccact
ccggccgcactatgcgttttttgtccttgctgccctcgattgccttccagcagcatgggctaaca
aagggagggtgtggggctcactcttaaggagcccatgaagcttacgttggataggaatggaaggg
caggaggggcgactggggcccgcccgccttcggagcacatgtccgacgccacctggatggggcga
ggcctgtggctttccgaagcaatcgggcgtgagtttagcctacctgggccatgtggccctagcac
tgggcacggtctggcctggcggtgccgcgttcccttgcctcccaacaagggtgaggccgtcccgc
ccggcaccagttgcttgcgcggaaagatggccgctcccggggccctgttgcaaggagctcaaaat
ggaggacgcggcagcccggtggagcggggggtgagtcacccacacaaaggaagagggccttgccc
ctcgccggccgctgcttcctgtgaccccgtggtctatcggccgcatagtcacctcgggcttctct
tgagcaccgctcgtcgcggcggggggaggggatctaatggcgttggagtttgttcacatttggtg
ggtggagactagtcaggccagcctggcgctggaagtcattcttggaatttgcccctttgagtttg
gagcgaggctaattctcaagcctcttagcggttcaaaggtattttctaaacccgtttccaggtgt
tgtgaaagccaccgctaattcaaagcaatccggagtaaattcaccatgtctagactggacaagag
caaagtcataaacggcgctctggaattactcaatggagtcggtatcgaaggcctgacgacaagga
aactcgctcaaaagctgggagttgagcagcctaccctgtactggcacgtgaagaacaagcgggcc
ctgctcgatgccctgccaatcgagatgctggacaggcatcatacccacttctgccccctggaagg
cgagtcatggcaagactttctgcggaacaacgccaagtcattccgctgtgctctcctctcacatc
gcgacggggctaaagtgcatctcggcacccgcccaacagagaaacagtacgaaaccctggaaaat
cagctcgcgttcctgtgtcagcaaggcttctccctggagaacgcactgtacgctctgtccgccgt
gggccactttacactgggctgcgtattggaggaacaggagcatcaagtagcaaaagaggaaagag
agacacctaccaccgattctatgcccccacttctgagacaagcaattgagctgttcgaccggcag
ggagccgaacctgccttccttttcggcctggaactaatcatatgtggcctggagaaacagctaaa
gtgcgaaagcgggggccggccgacgcccttgacgattttgacttagacatgctcccagccgatgc
ccttgacgactttgaccttgatatgctgcctgctgacgctcttgacgattttgaccttgacatgc
tccccgggtaaaattccgcccctctccctcccccccccctaacgttactggccgaagccgcttgg
aataaggccggtgtgcgtttgtctatatgttattttccaccatattgccgtcttttggcaatgtg
agggcccggaaacctggccctgtcttcttgacgagcattcctaggggtctttcccctctcgccaa
aggaatgcaaggtctgttgaatgtcgtgaaggaagcagttcctctggaagcttcttgaagacaaa
caacgtctgtagcgaccctttgcaggcagcggaaccccccacctggcgacaggtgcctctgcggc
caaaagccacgtgtataagatacacctgcaaaggcggcacaaccccagtgccacgttgtgagttg
gatagttgtggaaagagtcaaatggctctcctcaagcgtattcaacaaggggctgaaggatgccc
agaaggtaccccattgtatgggatctgatctggggcctcggtgcacatgctttacatgtgtttag
tcgaggttaaaaaacgtctaggccccccgaaccacggggacgtggttttcctttgaaaaacacga
tgataatatggccacaaccatgaccgagtacaagcccacggtgcgcctcgccacccgcgacgacg
tcccccgggccgtacgcaccctcgccgccgcgttcgccgactaccccgccacgcgccacaccgtc
gatccggaccgccacatcgagcgggtcaccgagctgcaagaactcttcctcacgcgcgtcgggct
cgacatcggcaaggtgtgggtcgcggacgacggcgccgcggtggcggtctggaccacgccggaga
gcgtcgaagcgggggcggtgttcgccgagatcggcccgcgcatggccgagttgagcggttcccgg
ctggccgcgcagcaacagatggaaggcctcctggcgccgcaccggcccaaggagcccgcgtggtt
cctggccaccgtcggcgtctcgcccgaccaccagggcaagggtctgggcagcgccgtcgtgctcc
ccggagtggaggggccgagcgcgccggggtgcccgccttcctggagacctccgcgccccgcaacc
tccccttctacgagcggctcggcttcaccgtcaccgccgacgtcgaggtgcccgaaggaccgcgc
acctggtgcatgacccgcaagcccggtgcctgacgcccgccccacgacccgcagcgcccgaccga
aaggagcgcacgaccccatgcatcgaaatgaccgaccaagcgaattcgctaggattatccctaat
acctgccaccccactcttaatcagtggtggaagaacggtctcagaactgtttgtttcaattggcc
atttaagtttagtagtaaaagactggttaatgataacaatgcatcgtaaaaccttcagaaggaaa
ggagaatgttttgtggaccactttggttttcttttttgcgtgtggcagttttaagttattagttt
ttaaaatcagtactttttaatggaaacaacttgaccaaaaatttgtcacagaattttgagaccca
ttaaaaaagttaaatgagaaacctgtgtgttcctttggtcaacaccgagacatttaggtgaaaga
catctaattctggttttacgaatctggaaacttcttgaaaatgtaattcttgagttaacacttct
gggtggagaatagggttgttttccccccacataattggaaggggaaggaatatcatttaaagcta
tgggagggttgctttgattacaacactggagagaaatgcagcatgttgctgattgcctgtcacta
aaacaggccaaaaactgagtccttgggttgcatagaaagctgccaaacgctagccaacaagctcg
tcatcgctttgcagaagagcagagaggatatgctcatcgtctaaagaactacccattttattata
tattagtcacgatatctataacaagaaaatatatatataataagttatcacgtaagtagaacatg
aaataacaatataattatcgtatgagttaaatcttaaaagtcacgtaaaagataatcatgcgtca
ttttgactcacgcggtcgttatagttcaaaatcagtgacacttaccgcattgacaagcacgcctc
acgggagctccaagcggcgactgagatgtcctaaatgcacagcgacggattcgcgctatttagaa
agagagagcaatatttcaagaatgcatgcgtcaattttacgcagactatctttctagggttaaaa
aagatttgcgctttactcgacctaaactttaaacacgtcatagaatcttcgtttgacaaaaacca
cattgtggccaagctgtgtgacgcgacgcgcgctaaagaatggcaaaccaagtcgcgcgaggtac
ccagcttttgttccctttagtgagggttaattccgagcttggcgtaatcatggtcatagctgttt
cctgtgtgaaattgttatccgctcacaattccacacaacatacgagccggaagcataaagtgtaa
agcctggggtgcctaatgagtgagctaactcacattaattgcgttgcgctcactgcccgctttcc
agtcgggaaacctgtcgtgccagctgcattaatgaatcggccaacgcgcggggagaggcggtttg
cgtattgggcgctcttccgcttcctcgctcactgactcgctgcgctcggtcgttcggctgcggcg
agcggtatcagctcactcaaaggcggtaatacggttatccacagaatcaggggataacgcaggaa
agaacatgtgagcaaaaggccagcaaaaggccaggaaccgtaaaaaggccgcgttgctggcgttt
ttccataggctccgcccccctgacgagcatcacaaaaatcgacgctcaagtcagaggtggcgaaa
cccgacaggactataaagataccaggcgtttccccctggaagctccctcgtgcgctctcctgttc
cgaccctgccgcttaccggatacctgtccgcctttctcccttcgggaagcgtggcgctttctcat
agctcacgctgtaggtatctcagttcggtgtaggtcgttcgctccaagctgggctgtgtgcacga
accccccgttcagcccgaccgctgcgccttatccggtaactatcgtcttgagtccaacccggtaa
gacacgacttatcgccactggcagcagccactggtaacaggattagcagagcgaggtatgtaggc
ggtgctacagagttcttgaagtggtggcctaactacggctacactagaaggacagtatttggtat
ctgcgctctgctgaagccagttaccttcggaaaaagagttggtagctcttgatccggcaaacaaa
ccaccgctggtagcggtggtttttttgtttgcaagcagcagattacgcgcagaaaaaaaggatct
caagaagatcctttgatcttttctacggggtctgacgctcagtggaacgaaaactcacgttaagg
gattttggtcatgagattatcaaaaaggatcttcacctagatccttttaaattaaaaatgaagtt
ttaaatcaatctaaagtatatatgagtaaacttggtctgacagttaccaatgcttaatcagtgag
gcacctatctcagcgatctgtctatttcgttcatccatagttgcctgactccccgtcgtgtagat
aactacgatacgggagggcttaccatctggccccagtgctgcaatgataccgcgagacccacgct
caccggctccagatttatcagcaataaaccagccagccggaagggccgagcgcagaagtggtcct
gcaactttatccgcctccatccagtctattaattgttgccgggaagctagagtaagtagttcgcc
agttaatagtttgcgcaacgttgttgccattgctacaggcatcgtggtgtcacgctcgtcgtttg
gtatggcttcattcagctccggttcccaacgatcaaggcgagttacatgatcccccatgttgtgc
aaaaaagcggttagctccttcggtcctccgatcgttgtcagaagtaagttggccgcagtgttatc
actcatggttatggcagcactgcataattctcttactgtcatgccatccgtaagatgcttttctg
tgactggtgagtactcaaccaagtcattctgagaatagtgtatgcggcgaccgagttgctcttgc
ccggcgtcaatacgggataataccgcgccacatagcagaactttaaaagtgctcatcattggaaa
acgttcttcggggcgaaaactctcaaggatcttaccgctgttgagatccagttcgatgtaaccca
ctcgtgcacccaactgatcttcagcatcttttactttcaccagcgtttctgggtgagcaaaaaca
ggaaggcaaaatgccgcaaaaaagggaataagggcgacacggaaatgttgaatactcatactctt
cctttttcaatattattgaagcatttatcagggttattgtctcatgagcggatacatatttgaat
gtatttagaaaaataaacaaataggggttccgcgcacatttccccgaaaagtgccacctg
(GATA6 CDS sequence 1)
SEQ ID NO: 3
Atggccttgactgacggcggctggtgcttgccgaagcgcttcggggccgcgggtgcggacgccag
cgactccagagcctttccagcgcgggagccctccacgccgccttcccccatctcttcctcgtcct
cctcctgctcccggggcggagagcggggccccggcggcgccagcaactgcgggacgcctcagctc
gacacggaggcggcggccggacccccggcccgctcgctgctgctcagttcctacgcttcgcatcc
cttcggggctccccacggaccttcggcgcctggggtcgcgggccccgggggcaacctgtcgagct
gggaggacttgctgctgttcactgacctcgaccaagccgcgaccgccagcaagctgctgtggtcc
agccgcggcgccaagctgagccccttcgcacccgagcagccggaggagatgtaccagaccctcgc
cgctctctccagccagggtccggccgcctacgacggcgcgcccggcggcttcgtgcactctgcgg
ccgcggcggcagcagccgcggcggggccagctccccggtctacgtgcccaccacccgcgtgggtt
ccatgctgcccggcctaccgtaccacctgcaggggtcgggcagtgggccagccaaccacgcgggc
ggcgcgggcgcgcaccccggctggcctcaggcctcggccgacagccctccatacggcagcggagg
cggcgcggctggcggcggggccgcggggcctggcggcgctggctcagccgcggcgcacgtctcgg
cgcgcttcccctactctcccagcccgcccatggccaacggcgccgcgcgggagccgggaggctac
gcggcggcgggcagtgggggcgcgggaggcgtgagcggcggcggcagtagcctggcggccatggg
cggccgcgagccccagtacagctcgctgtcggccgcgcggccgctgaacgggacgtaccaccacc
accaccaccaccaccaccaccatccgagcccctactcgccctacgtgggggcgccactgacgcct
gcctggcccgccggac
(GATA6 CDS sequence 2)
SEQ ID NO: 4
Ccttcgagaccccggtgctgcacagcctgcagagccgcgccggagccccgctcccggtgccccgg
ggtcccagt
(GATA6 CDS sequence 3)
SEQ ID NO: 5
Gacctgctggaggacctgtccgagagccgcgagtgcgtgaactgcggctccatccagacgccgct
gtggcggcgggacggcaccggccactacctgtgcaacgcctgcgggctctacagcaagatgaacg
gcctcagccggcccctcatcaagccgcagaagcg
(GATA6 CDS sequence 4)
SEQ ID NO: 6
Ccttcatcacggcggcttggattgtcctgtgccaactgtcacaccacaactaccaccttatggcg
cagaaacgccgagggtgaacccgtgtgcaatgcttgtggactctacatgaaactcca
(GATA6 CDS sequence 5)
SEQ ID NO: 7
Gtgcccagaccacttgctatgaaaaaagagggaattcaaaccaggaaacgaaaacctaagaacat
aaataaatcaaagacttgc
(GATA6 CDS sequence 6)
SEQ ID NO: 8
Gtaatagcaataattccattcccatgactccaacttccacctcttctaactcagatgattgcagc
aaaaatacttcccccacaacacaacctacagcct
(GATA6 CDS sequence 7)
SEQ ID NO: 9
gcgggtgccccggtgatgactggtgcgggagagagcaccaatcccgagaacagcgagctcaagta
ttcgggtcaagatgggctctacataggcgtcagtctcgcctcgccggccgaagtcacgtcctccg
tgcgaccggattcctggtgcgccctggccctggcc
(GATA6 CDS sequence included)
SEQ ID NO: 10
atggccttgactgacggcggctggtgcttgccgaagcgcttcggggccgcgggtgcggacgccag
cgactccagagcctttccagcgcgggagccctccacgccgccttcccccatctcttcctcgtcct
cctcctgctcccggggggagagcggggccccggcggcgccagcaactgcgggacgcctcagctcg
acacggaggcggcggccggacccccggcccgctcgctgctgctcagttcctacgcttcgcatccc
ttcggggctccccacggaccttcggcgcctggggtcgcgggccccgggggcaacctgtcgagctg
ggaggacttgctgctgttcactgacctcgaccaagccgcgaccgccagcaagctgctgtggtcca
gccgcggcgccaagctgagccccttcgcacccgagcagccggaggagatgtaccagaccctcgcc
gctctctccagccagggtccggccgcctacgacggcgcgcccggcggcttcgtgcactctgcggc
cgcggcggcagcagccgcggcggcggccagctccccggtctacgtgcccaccacccgcgtgggtt
ccatgctgcccggcctaccgtaccacctgcaggggtcgggcagtgggccagccaaccacgcgggc
ggcgcgggcgcgcaccccggctggcctcaggcctcggccgacagccctccatacggcagcggagg
cggcgcggctggcggcggggccgcggggcctggcggcgctggctcagccgcggcgcacgtctcgg
cgcgcttcccctactctcccagcccgcccatggccaacggcgccgcgcgggagccgggaggctac
gcggcggcgggcagtgggggcgcgggaggcgtgagcggcggcggcagtagcctggcggccatggg
cggccgcgagccccagtacagctcgctgtcggccgcgcggccgctgaacgggacgtaccaccacc
accaccaccaccaccaccaccatccgagcccctactcgccctacgtgggggcgccactgacgcct
gcctggcccgccggacccttcgagaccccggtgctgcacagcctgcagagccgcgccggagcccc
gctcccggtgccccggggtcccagtgcagacctgctggaggacctgtccgagagccgcgagtgcg
tgaactgcggctccatccagacgccgctgtggcggcgggacggcaccggccactacctgtgcaac
gcctgcgggctctacagcaagatgaacggcctcagccggcccctcatcaagccgcagaagcgcgt
gccttcatcacggcggcttggattgtcctgtgccaactgtcacaccacaactaccaccttatggc
gcagaaacgccgagggtgaacccgtgtgcaatgcttgtggactctacatgaaactccatggggtg
cccagaccacttgctatgaaaaaagagggaattcaaaccaggaaacgaaaacctaagaacataaa
taaatcaaagacttgctctggtaatagcaataattccattcccatgactccaacttccacctctt
ctaactcagatgattgcagcaaaaatacttcccccacaacacaacctacagcctcaggggggggc
cccggtgatgactggtgcgggagagagcaccaatcccgagaacagcgagctcaagtattcgggtc
aagatgggctctacataggcgtcagtctcgcctcgccggccgaagtcacgtcctccgtgcgaccg
gattcctggtgcgccctggccctggcc
(tet operator)
SEQ ID NO: 11
tccctatcagtgatagaga
(OCT4 polypeptide sequence)
SEQ ID NO: 12
MAGHLASDFAFSPPPGGGGDGPGGPEPGWVDPRTWLSFQGPPGGPGIGPGVGPGSEVWGIPPCPP
PYEFCGGMAYCGPQVGVGLVPQGGLETSQPEGEAGVGVESNSDGASPEPCTVTPGAVKLEKEKLE
QNPEESQDIKALQKELEQFAKLLKQKRITLGYTQADVGLTLGVLFGKVFSQTTICRFEALQLSFK
NMCKLRPLLQKWVEEADNNENLQEICKAETLVQARKRKRTSIENRVRGNLENLFLQCPKPTLQQI
SHIAQQLGLEKDVVRVWFCNRRQKGKRSSSDYAQREDFEAAGSPFSGGPVSFPLAPGPHFGTPGY
GSPHFTALYSSVPFPEGEAFPPVSVTTLGSPMHSN
(GATA4 polypeptide sequence)
SEQ ID NO: 13
MYQSLAMAANHGPPPGAYEAGGPGAFMHGAGAASSPVYVPTPRVPSSVLGLSYLQGGGAGSASGG
ASGGSSGGAASGAGPGTQQGSPGWSQAGADGAAYTPPPVSPRFSFPGTTGSLAAAAAAAAAREAA
AYSSGGGAAGAGLAGREQYGRAGFAGSYSSPYPAYMADVGASWAAAAAASAGPFDSPVLHSLPGR
ANPAARHPNLDMEDDFSEGRECVNCGAMSTPLWRRDGTGHYLCNACGLYHKMNGINRPLIKPQRR
LSASRRVGLSCANCQTTTTTLWRRNAEGEPVCNACGLYMKLHGVPRPLAMRKEGIQTRKRKPKNL
NKSKTPAAPSGSESLPPASGASSNSSNATTSSSEEMRPIKTEPGLSSHYGHSSSVSQTFSVSAMS
GHGPSIHPVLSALKLSPQGYASPVSQSPQTSSKQDSWNSLVLADSHGDIITA
(FMorg30 PB-TAG-ERP2 #80479 - pENTR_L1_ETV2_L2)
SEQ ID NO: 14
GGAAATTGTAAACGTTAATATTTTGTTAAAATTCGCGTTAAATTTTTGTTAAATCAGCTCATTTT
TTAACCAATAGGCCGAAATCGGCAAAATCCCTTATAAATCAAAAGAATAGACCGAGATAGGGTTG
AGTGTTGTTCCAGTTTGGAACAAGAGTCCACTATTAAAGAACGTGGACTCCAACGTCAAAGGGCG
AAAAACCGTCTATCAGGGCGATGGCCCACTACGTGAACCATCACCCTAATCAAGTTTTTTGGGGT
CGAGGTGCCGTAAAGCACTAAATCGGAACCCTAAAGGGAGCCCCCGATTTAGAGCTTGACGGGGA
AAGCCGGCGAACGTGGCGAGAAAGGAAGGGAAGAAAGCGAAAGGAGCGGGCGCTAGGGCGCTGGC
AAGTGTAGCGGTCACGCTGCGCGTAACCACCACACCCGCCGCGCTTAATGCGCCGCTACAGGGCG
CGTCGCGCCATTCGCCATTCAGGCTGCGCAACTGTTGGGAAGGGCGATCGGTGCGGGCCTCTTCG
CTATTACGCCAGCTGGCGAAAGGGGGATGTGCTGCAAGGCGATTAAGTTGGGTAACGCCAGGGTT
TTCCCAGTCACGACGTTGTAAAACGACGGCCAGTGAATTGTAATACGACTCACTATAGGGCGAAT
TGGAGCTCGGTATTCACGACAGCAGGCTGAATAATAAAAAAATTAGAAACTATTATTTAACCCTA
GAAAGATAATCATATTGTGACGTACGTTAAAGATAATCATGCGTAAAATTGACGCATGTGTTTTA
TCGGTCTGTATATCGAGGTTTATTTATTAATTTGAATAGATATTAAGTTTTATTATATTTACACT
TACATACTAATAATAAATTCAACAAACAATTTATTTATGTTTATTTATTTATTAAAAAAAAACAA
AAACTCAAAATTTCTTCTATAAAGTAACAAAACTTTTAAACATTCTCTCTTTTACAAAAATAAAC
TTATTTTGTACTTTAAAAACAGTCATGTTGTATTATAAAATAAGTAATTAGCTTAACCTATACAT
AATAGAAACAAATTATACTTATTAGTCAGTCAGAAACAACTTTGGCACATATCAATATTATGCTC
TCGTTAATTAACTCGAGGGATCCAAGCTGGCCGCACGTCTCCCTATCAGTGATAGAGAAGTCGAC
ACGTCTCGAGCTCCCTATCAGTGATAGAGAAGGTACGTCTAGAACGTCTCCCTATCAGTGATAGA
GAAGTCGACACGTCTCGAGCTCCCTATCAGTGATAGAGAAGGTACGTCTAGAACGTCTCCCTATC
AGTGATAGAGAAGTCGACACGTCTCGAGCTCCCTATCAGTGATAGAGAAGGTACGTCTAGAACGT
CTCCCTATCAGTGATAGAGAAGTCGACACGTCTCGAGCTCCCTATCAGTGATAGAGAAGGTACCC
CCTATATAAGCAGAGCTCGTTTAGTGAACCGTCAGATCGCCTGGAGACGCCATCCACGCTGTTTT
GACCTCCATAGAAGACACCGGGACCGATCCAGCCTCCGCGGTGGCGGCCATCACAAGTTTGTACA
AAAAAGCAGGCTGATTACCGGTTGCCACCGTACAAAAAAGCAGAAGGGCCGTCAAGGCCCACCAT
GGACCTGTGGAACTGGGATGAGGCATCCCCACAGGAAGTGCCTCCAGGGAACAAGCTGGCAGGGC
TTGAAGGAGCCAAATTAGGCTTCTGTTTCCCTGATCTGGCACTCCAAGGGGACACGCCGACAGCG
ACAGCAGAGACATGCTGGAAAGGTACAAGCTCATCCCTGGCAAGCTTCCCACAGCTGGACTGGGG
CTCCGCGTTACTGCACCCAGAAGTTCCATGGGGGGCGGAGCCCGACTCTCAGGCTCTTCCGTGGT
CCGGGGACTGGACAGACATGGCGTGCACAGCCTGGGACTCTTGGAGCGGCGCCTCGCAGACCCTG
GGCCCCGCCCCTCTCGGCCCGGGCCCCATCCCCGCCGCCGGCTCCGAAGGCGCCGCGGGCCAGAA
CTGCGTCCCCGTGGCGGGAGAGGCCACCTCGTGGTCGCGCGCCCAGGCCGCCGGGAGCAACACCA
GCTGGGACTGTTCTGTGGGGCCCGACGGCGATACCTACTGGGGCAGTGGCCTGGGCGGGGAGCCG
CGCACGGACTGTACCATTTCGTGGGGCGGGCCCGCGGGCCCGGACTGTACCACCTCCTGGAACCC
GGGGCTGCATGCGGGTGGCACCACCTCTTTGAAGCGGTACCAGAGCTCAGCTCTCACCGTTTGCT
CCGAACCGAGCCCGCAGTCGGACCGTGCCAGTTTGGCTCGATGCCCCAAAACTAACCACCGAGGT
CCCATTCAGCTGTGGCAGTTCCTCCTGGAGCTGCTCCACGACGGGGCGCGTAGCAGCTGCATCCG
TTGGACTGGCAACAGCCGCGAGTTCCAGCTGTGCGACCCCAAAGAGGTGGCTCGGCTGTGGGGCG
AGCGCAAGAGAAAGCCGGGCATGAATTACGAGAAGCTGAGCCGGGGCCTTCGCTACTACTATCGC
CGCGACATCGTGCGCAAGAGCGGGGGGCGAAAGTACACGTACCGCTTCGGGGGCCGCGTGCCCAG
CCTAGCCTATCCGGACTGTGCGGGAGGCGGACGGGGAGCAGAGACACAATAGGGCCTCATGGGCC
CAGCTTTCTTGTACGAATTCTGCAGTCGACGGTACCGCGGGCCCGGGATCCACCGGATCTAGATA
ACTGATCTACCCAGCTTTCTTGTACAAAGTGGTGATCTAGCTAAAACGCGGCCTCGAATCGAATT
CCGCCCCCCCCCCTCTCCCTCCCCCCCCCCTAACGTTACTGGCCGAAGCCGCTTGGAATAAGGCC
GGTGTGCGTTTGTCTATATGTTATTTTCCACCATATTGCCGTCTTTTGGCAATGTGAGGGCCCGG
AAACCTGGCCCTGTCTTCTTGACGAGCATTCCTAGGGGTCTTTCCCCTCTCGCCAAAGGAATGCA
AGGTCTGTTGAATGTCGTGAAGGAAGCAGTTCCTCTGGAAGCTTCTTGAAGACAAACAACGTCTG
TAGCGACCCTTTGCAGGCAGCGGAACCCCCCACCTGGCGACAGGTGCCTCTGCGGCCAAAAGCCA
CGTGTATAAGATACACCTGCAAAGGCGGCACAACCCCAGTGCCACGTTGTGAGTTGGATAGTTGT
GGAAAGAGTCAAATGGCTCTCCTCAAGCGTATTCAACAAGGGGCTGAAGGATGCCCAGAAGGTAC
CCCATTGTATGGGATCTGATCTGGGGCCTCGGTGCACATGCTTTACATGTGTTTAGTCGAGGTTA
AAAAACGTCTAGGCCCCCCGAACCACGGGGACGTGGTTTTCCTTTGAAAAACACGATGATAATAT
GGCCACAACCATGGTGAGCAAGGGCGAGGAGCTGTTCACCGGGGTGGTGCCCATCCTGGTCGAGC
TGGACGGCGACGTAAACGGCCACAAGTTCAGCGTGTCCGGCGAGGGCGAGGGCGATGCCACCTAC
GGCAAGCTGACCCTGAAGTTCATCTGCACCACCGGCAAGCTGCCCGTGCCCTGGCCCACCCTCGT
GACCACCCTGACCTACGGCGTGCAGTGCTTCAGCCGCTACCCCGACCACATGAAGCAGCACGACT
TCTTCAAGTCCGCCATGCCCGAAGGCTACGTCCAGGAGCGCACCATCTTCTTCAAGGACGACGGC
AACTACAAGACCCGCGCCGAGGTGAAGTTCGAGGGCGACACCCTGGTGAACCGCATCGAGCTGAA
GGGCATCGACTTCAAGGAGGACGGCAACATCCTGGGGCACAAGCTGGAGTACAACTACAACAGCC
ACAACGTCTATATCATGGCCGACAAGCAGAAGAACGGCATCAAGGTGAACTTCAAGATCCGCCAC
AACATCGAGGACGGCAGCGTGCAGCTCGCCGACCACTACCAGCAGAACACCCCCATCGGCGACGG
CCCCGTGCTGCTGCCCGACAACCACTACCTGAGCACCCAGTCCGCCCTGAGCAAAGACCCCAACG
AGAAGCGCGATCACATGGTCCTGCTGGAGTTCGTGACCGCCGCCGGGATCACTCTCGGCATGGAC
GAGCTGTACAAGTAAAGCGGCCGCGATCTTTTTCCCTCTGCCAAAAATTATGGGGACATCATGAA
GCCCCTTGAGCATCTGACTTCTGGCTAATAAAGGAAATTTATTTTCATTGCAATAGTGTGTTGGA
ATTTTTTGTGTCTCTCACTCGGAAGGACATATGGGAGGGCAAATCATTTAAAACATCAGAATGAG
TATTTGGTTTAGAGTTTGGCAACATATGCCATATGCTGGCTGCCATGAACAAAGGTGGCTATAAA
GAGGTCATCAGTATATGAAACAGCCCCCTGCTGTCCATTCCTTATTCCATAGAAAAGCCTTGACT
TGAGGTTAGATTTTTTTTATATTTTGTTTTGTGTTATTTTTTTCTTTAACATCCCTAAAATTTTC
CTTACATGTTTTACTAGCCAGATTTTTCCTCCTCTCCTGACTACTCCCAGTCATAGCTGTCCCTC
TTCTCTTATGAAGATCCCTCGACCTGCAGGAATTCGATTACTTAAGCCTAGGATCCAGATCTGTA
CATTCGAATCGATGAATTCGTTAACCATGGAAGCTTCTGCAGCGGCCGCACTAGCGGTGGCGGCC
TCGACATTGATTATTGACTAGTTATTAATAGTAATCAATTACGGGGTCATTAGTTCATAGCCCAT
ATATGGAGTTCCGCGTTACATAACTTACGGTAAATGGCCCGCCTGGCTGACCGCCCAACGACCCC
CGCCCATTGACGTCAATAATGACGTATGTTCCCATAGTAACGCCAATAGGGACTTTCCATTGACG
TCAATGGGTGGAGTATTTACGGTAAACTGCCCACTTGGCAGTACATCAAGTGTATCATATGCCAA
GTACGCCCCCTATTGACGTCAATGACGGTAAATGGCCCGCCTGGCATTATGCCCAGTACATGACC
TTATGGGACTTTCCTACTTGGCAGTACATCTACGTATTAGTCATCGCTATTACCATGATGATGCG
GTTTTGGCAGTACATCAATGGGCGTGGATAGCGGTTTGACTCACGGGGATTTCCAAGTCTCCACC
CCATTGACGTCAATGGGAGTTTGTTTTGACTAGTGGAGCCGAGAGTAATTCATACAAAAGGAGGG
ATCGCCTTCGCAAGGGGAGAGCCCAGGGACCGTCCCTAAATTCTCACAGACCCAAATCCCTGTAG
CCGCCCCACGACAGCGCGAGGAGCATGCGCCCAGGGCTGAGCGCGGGTAGATCAGAGCACACAAG
CTCACAGTCCCCGGCGGTGGGGGGAGGGGCGCGCTGAGCGGGGGCCAGGGAGCTGGCGCGGGGCA
AACTGGGAAAGTGGTGTCGTGTGCTGGCTCCGCCCTCTTCCCGAGGGTGGGGGAGAACGGTATAT
AAGTGCGGTAGTCGCCTTGGACGTTCTTTTTCGCAACGGGTTTGCCGTCAGAACGCAGGTGAGTG
GCGGGTGTGGCTTCCGCGGGCCCCGGAGCTGGAGCCCTGCTCTGAGCGGGCCGGGCTGATATGCG
AGTGTCGTCCGCAGGGTTTAGCTGTGAGCATTCCCACTTCGAGTGGCGGGCGGTGCGGGGGTGAG
AGTGCGAGGCCTAGCGGCAACCCCGTAGCCTCGCCTCGTGTCCGGCTTGAGGCCTAGCGTGGTGT
CCGCCGCCGCGTGCCACTCCGGCCGCACTATGCGTTTTTTGTCCTTGCTGCCCTCGATTGCCTTC
CAGCAGCATGGGCTAACAAAGGGAGGGTGTGGGGCTCACTCTTAAGGAGCCCATGAAGCTTACGT
TGGATAGGAATGGAAGGGCAGGAGGGGCGACTGGGGCCCGCCCGCCTTCGGAGCACATGTCCGAC
GCCACCTGGATGGGGCGAGGCCTGTGGCTTTCCGAAGCAATCGGGCGTGAGTTTAGCCTACCTGG
GCCATGTGGCCCTAGCACTGGGCACGGTCTGGCCTGGCGGTGCCGCGTTCCCTTGCCTCCCAACA
AGGGTGAGGCCGTCCCGCCCGGCACCAGTTGCTTGCGCGGAAAGATGGCCGCTCCCGGGGCCCTG
TTGCAAGGAGCTCAAAATGGAGGACGCGGCAGCCCGGTGGAGCGGGCGGGTGAGTCACCCACACA
AAGGAAGAGGGCCTTGCCCCTCGCCGGCCGCTGCTTCCTGTGACCCCGTGGTCTATCGGCCGCAT
AGTCACCTCGGGCTTCTCTTGAGCACCGCTCGTCGCGGCGGGGGGAGGGGATCTAATGGCGTTGG
AGTTTGTTCACATTTGGTGGGTGGAGACTAGTCAGGCCAGCCTGGCGCTGGAAGTCATTCTTGGA
ATTTGCCCCTTTGAGTTTGGAGCGAGGCTAATTCTCAAGCCTCTTAGCGGTTCAAAGGTATTTTC
TAAACCCGTTTCCAGGTGTTGTGAAAGCCACCGCTAATTCAAAGCAATCCGGAGTAAATTCACCA
TGTCTAGACTGGACAAGAGCAAAGTCATAAACGGCGCTCTGGAATTACTCAATGGAGTCGGTATC
GAAGGCCTGACGACAAGGAAACTCGCTCAAAAGCTGGGAGTTGAGCAGCCTACCCTGTACTGGCA
CGTGAAGAACAAGCGGGCCCTGCTCGATGCCCTGCCAATCGAGATGCTGGACAGGCATCATACCC
ACTTCTGCCCCCTGGAAGGCGAGTCATGGCAAGACTTTCTGCGGAACAACGCCAAGTCATTCCGC
TGTGCTCTCCTCTCACATCGCGACGGGGCTAAAGTGCATCTCGGCACCCGCCCAACAGAGAAACA
GTACGAAACCCTGGAAAATCAGCTCGCGTTCCTGTGTCAGCAAGGCTTCTCCCTGGAGAACGCAC
TGTACGCTCTGTCCGCCGTGGGCCACTTTACACTGGGCTGCGTATTGGAGGAACAGGAGCATCAA
GTAGCAAAAGAGGAAAGAGAGACACCTACCACCGATTCTATGCCCCCACTTCTGAGACAAGCAAT
TGAGCTGTTCGACCGGCAGGGAGCCGAACCTGCCTTCCTTTTCGGCCTGGAACTAATCATATGTG
GCCTGGAGAAACAGCTAAAGTGCGAAAGCGGCGGGCCGGCCGACGCCCTTGACGATTTTGACTTA
GACATGCTCCCAGCCGATGCCCTTGACGACTTTGACCTTGATATGCTGCCTGCTGACGCTCTTGA
CGATTTTGACCTTGACATGCTCCCCGGGTAAAATTCCGCCCCTCTCCCTCCCCCCCCCCTAACGT
TACTGGCCGAAGCCGCTTGGAATAAGGCCGGTGTGCGTTTGTCTATATGTTATTTTCCACCATAT
TGCCGTCTTTTGGCAATGTGAGGGCCCGGAAACCTGGCCCTGTCTTCTTGACGAGCATTCCTAGG
GGTCTTTCCCCTCTCGCCAAAGGAATGCAAGGTCTGTTGAATGTCGTGAAGGAAGCAGTTCCTCT
GGAAGCTTCTTGAAGACAAACAACGTCTGTAGCGACCCTTTGCAGGCAGCGGAACCCCCCACCTG
GCGACAGGTGCCTCTGCGGCCAAAAGCCACGTGTATAAGATACACCTGCAAAGGCGGCACAACCC
CAGTGCCACGTTGTGAGTTGGATAGTTGTGGAAAGAGTCAAATGGCTCTCCTCAAGCGTATTCAA
CAAGGGGCTGAAGGATGCCCAGAAGGTACCCCATTGTATGGGATCTGATCTGGGGCCTCGGTGCA
CATGCTTTACATGTGTTTAGTCGAGGTTAAAAAACGTCTAGGCCCCCCGAACCACGGGGACGTGG
TTTTCCTTTGAAAAACACGATGATAATATGGCCACAACCATGACCGAGTACAAGCCCACGGTGCG
CCTCGCCACCCGCGACGACGTCCCCCGGGCCGTACGCACCCTCGCCGCCGCGTTCGCCGACTACC
CCGCCACGCGCCACACCGTCGATCCGGACCGCCACATCGAGCGGGTCACCGAGCTGCAAGAACTC
TTCCTCACGCGCGTCGGGCTCGACATCGGCAAGGTGTGGGTCGCGGACGACGGCGCCGCGGTGGC
GGTCTGGACCACGCCGGAGAGCGTCGAAGCGGGGGCGGTGTTCGCCGAGATCGGCCCGCGCATGG
CCGAGTTGAGCGGTTCCCGGCTGGCCGCGCAGCAACAGATGGAAGGCCTCCTGGCGCCGCACCGG
CCCAAGGAGCCCGCGTGGTTCCTGGCCACCGTCGGCGTCTCGCCCGACCACCAGGGCAAGGGTCT
GGGCAGCGCCGTCGTGCTCCCCGGAGTGGAGGCGGCCGAGCGCGCCGGGGTGCCCGCCTTCCTGG
AGACCTCCGCGCCCCGCAACCTCCCCTTCTACGAGCGGCTCGGCTTCACCGTCACCGCCGACGTC
GAGGTGCCCGAAGGACCGCGCACCTGGTGCATGACCCGCAAGCCCGGTGCCTGACGCCCGCCCCA
CGACCCGCAGCGCCCGACCGAAAGGAGCGCACGACCCCATGCATCGAAATGACCGACCAAGCGAA
TTCGCTAGGATTATCCCTAATACCTGCCACCCCACTCTTAATCAGTGGTGGAAGAACGGTCTCAG
AACTGTTTGTTTCAATTGGCCATTTAAGTTTAGTAGTAAAAGACTGGTTAATGATAACAATGCAT
CGTAAAACCTTCAGAAGGAAAGGAGAATGTTTTGTGGACCACTTTGGTTTTCTTTTTTGCGTGTG
GCAGTTTTAAGTTATTAGTTTTTAAAATCAGTACTTTTTAATGGAAACAACTTGACCAAAAATTT
GTCACAGAATTTTGAGACCCATTAAAAAAGTTAAATGAGAAACCTGTGTGTTCCTTTGGTCAACA
CCGAGACATTTAGGTGAAAGACATCTAATTCTGGTTTTACGAATCTGGAAACTTCTTGAAAATGT
AATTCTTGAGTTAACACTTCTGGGTGGAGAATAGGGTTGTTTTCCCCCCACATAATTGGAAGGGG
AAGGAATATCATTTAAAGCTATGGGAGGGTTGCTTTGATTACAACACTGGAGAGAAATGCAGCAT
GTTGCTGATTGCCTGTCACTAAAACAGGCCAAAAACTGAGTCCTTGGGTTGCATAGAAAGCTGCC
AAACGCTAGCCAACAAGCTCGTCATCGCTTTGCAGAAGAGCAGAGAGGATATGCTCATCGTCTAA
AGAACTACCCATTTTATTATATATTAGTCACGATATCTATAACAAGAAAATATATATATAATAAG
TTATCACGTAAGTAGAACATGAAATAACAATATAATTATCGTATGAGTTAAATCTTAAAAGTCAC
GTAAAAGATAATCATGCGTCATTTTGACTCACGCGGTCGTTATAGTTCAAAATCAGTGACACTTA
CCGCATTGACAAGCACGCCTCACGGGAGCTCCAAGCGGCGACTGAGATGTCCTAAATGCACAGCG
ACGGATTCGCGCTATTTAGAAAGAGAGAGCAATATTTCAAGAATGCATGCGTCAATTTTACGCAG
ACTATCTTTCTAGGGTTAAAAAAGATTTGCGCTTTACTCGACCTAAACTTTAAACACGTCATAGA
ATCTTCGTTTGACAAAAACCACATTGTGGCCAAGCTGTGTGACGCGACGCGCGCTAAAGAATGGC
AAACCAAGTCGCGCGAGGTACCCAGCTTTTGTTCCCTTTAGTGAGGGTTAATTCCGAGCTTGGCG
TAATCATGGTCATAGCTGTTTCCTGTGTGAAATTGTTATCCGCTCACAATTCCACACAACATACG
AGCCGGAAGCATAAAGTGTAAAGCCTGGGGTGCCTAATGAGTGAGCTAACTCACATTAATTGCGT
TGCGCTCACTGCCCGCTTTCCAGTCGGGAAACCTGTCGTGCCAGCTGCATTAATGAATCGGCCAA
CGCGCGGGGAGAGGCGGTTTGCGTATTGGGCGCTCTTCCGCTTCCTCGCTCACTGACTCGCTGCG
CTCGGTCGTTCGGCTGCGGCGAGCGGTATCAGCTCACTCAAAGGCGGTAATACGGTTATCCACAG
AATCAGGGGATAACGCAGGAAAGAACATGTGAGCAAAAGGCCAGCAAAAGGCCAGGAACCGTAAA
AAGGCCGCGTTGCTGGCGTTTTTCCATAGGCTCCGCCCCCCTGACGAGCATCACAAAAATCGACG
CTCAAGTCAGAGGTGGCGAAACCCGACAGGACTATAAAGATACCAGGCGTTTCCCCCTGGAAGCT
CCCTCGTGCGCTCTCCTGTTCCGACCCTGCCGCTTACCGGATACCTGTCCGCCTTTCTCCCTTCG
GGAAGCGTGGCGCTTTCTCATAGCTCACGCTGTAGGTATCTCAGTTCGGTGTAGGTCGTTCGCTC
CAAGCTGGGCTGTGTGCACGAACCCCCCGTTCAGCCCGACCGCTGCGCCTTATCCGGTAACTATC
GTCTTGAGTCCAACCCGGTAAGACACGACTTATCGCCACTGGCAGCAGCCACTGGTAACAGGATT
AGCAGAGCGAGGTATGTAGGCGGTGCTACAGAGTTCTTGAAGTGGTGGCCTAACTACGGCTACAC
TAGAAGGACAGTATTTGGTATCTGCGCTCTGCTGAAGCCAGTTACCTTCGGAAAAAGAGTTGGTA
GCTCTTGATCCGGCAAACAAACCACCGCTGGTAGCGGTGGTTTTTTTGTTTGCAAGCAGCAGATT
ACGCGCAGAAAAAAAGGATCTCAAGAAGATCCTTTGATCTTTTCTACGGGGTCTGACGCTCAGTG
GAACGAAAACTCACGTTAAGGGATTTTGGTCATGAGATTATCAAAAAGGATCTTCACCTAGATCC
TTTTAAATTAAAAATGAAGTTTTAAATCAATCTAAAGTATATATGAGTAAACTTGGTCTGACAGT
TACCAATGCTTAATCAGTGAGGCACCTATCTCAGCGATCTGTCTATTTCGTTCATCCATAGTTGC
CTGACTCCCCGTCGTGTAGATAACTACGATACGGGAGGGCTTACCATCTGGCCCCAGTGCTGCAA
TGATACCGCGAGACCCACGCTCACCGGCTCCAGATTTATCAGCAATAAACCAGCCAGCCGGAAGG
GCCGAGCGCAGAAGTGGTCCTGCAACTTTATCCGCCTCCATCCAGTCTATTAATTGTTGCCGGGA
AGCTAGAGTAAGTAGTTCGCCAGTTAATAGTTTGCGCAACGTTGTTGCCATTGCTACAGGCATCG
TGGTGTCACGCTCGTCGTTTGGTATGGCTTCATTCAGCTCCGGTTCCCAACGATCAAGGCGAGTT
ACATGATCCCCCATGTTGTGCAAAAAAGCGGTTAGCTCCTTCGGTCCTCCGATCGTTGTCAGAAG
TAAGTTGGCCGCAGTGTTATCACTCATGGTTATGGCAGCACTGCATAATTCTCTTACTGTCATGC
CATCCGTAAGATGCTTTTCTGTGACTGGTGAGTACTCAACCAAGTCATTCTGAGAATAGTGTATG
CGGCGACCGAGTTGCTCTTGCCCGGCGTCAATACGGGATAATACCGCGCCACATAGCAGAACTTT
AAAAGTGCTCATCATTGGAAAACGTTCTTCGGGGCGAAAACTCTCAAGGATCTTACCGCTGTTGA
GATCCAGTTCGATGTAACCCACTCGTGCACCCAACTGATCTTCAGCATCTTTTACTTTCACCAGC
GTTTCTGGGTGAGCAAAAACAGGAAGGCAAAATGCCGCAAAAAAGGGAATAAGGGCGACACGGAA
ATGTTGAATACTCATACTCTTCCTTTTTCAATATTATTGAAGCATTTATCAGGGTTATTGTCTCA
TGAGCGGATACATATTTGAATGTATTTAGAAAAATAAACAAATAGGGGTTCCGCGCACATTTCCC
CGAAAAGTGCCACCTG
(ETV2 CDS)
SEQ ID NO: 15
ATGGACCTGTGGAACTGGGATGAGGCATCCCCACAGGAAGTGCCTCCAGGGAACAAGCTGGCAGG
GCTTGAAGGAGCCAAATTAGGCTTCTGTTTCCCTGATCTGGCACTCCAAGGGGACACGCCGACAG
CGACAGCAGAGACATGCTGGAAAGGTACAAGCTCATCCCTGGCAAGCTTCCCACAGCTGGACTGG
GGCTCCGCGTTACTGCACCCAGAAGTTCCATGGGGGGCGGAGCCCGACTCTCAGGCTCTTCCGTG
GTCCGGGGACTGGACAGACATGGCGTGCACAGCCTGGGACTCTTGGAGCGGCGCCTCGCAGACCC
TGGGCCCCGCCCCTCTCGGCCCGGGCCCCATCCCCGCCGCCGGCTCCGAAGGCGCCGCGGGCCAG
AACTGCGTCCCCGTGGCGGGAGAGGCCACCTCGTGGTCGCGCGCCCAGGCCGCCGGGAGCAACAC
CAGCTGGGACTGTTCTGTGGGGCCCGACGGCGATACCTACTGGGGCAGTGGCCTGGGCGGGGAGC
CGCGCACGGACTGTACCATTTCGTGGGGGGGGCCCGCGGGCCCGGACTGTACCACCTCCTGGAAC
CCGGGGCTGCATGCGGGTGGCACCACCTCTTTGAAGCGGTACCAGAGCTCAGCTCTCACCGTTTG
CTCCGAACCGAGCCCGCAGTCGGACCGTGCCAGTTTGGCTCGATGCCCCAAAACTAACCACCGAG
GTCCCATTCAGCTGTGGCAGTTCCTCCTGGAGCTGCTCCACGACGGGGCGCGTAGCAGCTGCATC
CGTTGGACTGGCAACAGCCGCGAGTTCCAGCTGTGCGACCCCAAAGAGGTGGCTCGGCTGTGGGG
CGAGCGCAAGAGAAAGCCGGGCATGAATTACGAGAAGCTGAGCCGGGGCCTTCGCTACTACTATC
GCCGCGACATCGTGCGCAAGAGCGGGGGGCGAAAGTACACGTACCGCTTCGGGGGCCGCGTGCCC
AGCCTAGCCTATCCGGACTGTGCGGGAGGCGGACGGGGAGCAGAGACACAATAG
(ETV2 polypeptide sequence)
SEQ ID NO: 16
MDLWNWDEASPQEVPPGNKLAGLEGAKLGFCFPDLALQGDTPTATAETCWKGTSSSLASFPQLDW
GSALLHPEVPWGAEPDSQALPWSGDWTDMACTAWDSWSGASQTLGPAPLGPGPIPAAGSEGAAGQ
NCVPVAGEATSWSRAQAAGSNTSWDCSVGPDGDTYWGSGLGGEPRTDCTISWGGPAGPDCTTSWN
PGLHAGGTTSLKRYQSSALTVCSEPSPQSDRASLARCPKTNHRGPIQLWQFLLELLHDGARSSCI
RWTGNSREFQLCDPKEVARLWGERKRKPGMNYEKLSRGLRYYYRRDIVRKSGGRKYTYRFGGRVP
SLAYPDCAGGGRGAETQ

1. A method of ex vivo or in vitro generating a cellular mass that comprises one or more embryonic cell structures and one or more extraembryonic cell structures, comprising
a) obtaining one or more of a first group of stem cells comprising a first inducible transgene encoding a GATA binding protein 6 (GATA6) polypeptide;
b) contacting the one or more first group of stem cells with an inducing agent that induces an expression of the transgene encoding a GATA6 polypeptide;
c) mixing the one or more first groups of stem cells of step b) with a second group of stem cells; and
d) culturing the mixture of cells of step c) on a surface for at least 7 days, thereby producing the cellular mass.

2. The method of claim 1, wherein the second group of stem cells do not have the inducible transgene encoding the GATA6 polypeptide.

3. The method of claim 1, wherein the second group of stem cells comprise an inducible transgene encoding an ETS variant transcription factor 2 (ETV2) polypeptide, and further comprising contacting the second group of stem cells with an inducing agent that induces an expression of the transgene encoding the ETV2 polypeptide.

4. The method of claim 1, wherein step c) further comprises contacting the one or more first groups of stem cells of step b) with the second group of stem cells and a third group of cells, wherein the third group of cells comprise the inducible transgene encoding an ETV2 polypeptide, and further comprising contacting the third group of stem cells with an inducing agent that induces an expression of the transgene encoding the ETV2 polypeptide.

5. (canceled)

6. The method of claim 1, wherein the one or more first groups of cells and the second group of stem cells are induced pluripotent stem cells (iPSCs).

7. (canceled)

8. (canceled)

9. The method of claim 1, wherein the transgene encoding the GATA6 polypeptide is at least about 80% identical to a sequence selected from the group consisting of SEQ ID NOs: 3-10 or a fragment thereof.

10. The method of claim 1, wherein the one or more first groups of stem cells comprise one or more nucleic acid sequences at least about 80% identical to the sequence of SEQ ID NO: 1 or SEQ ID NO: 2 or a fragment thereof.

11. The method of claim 1, wherein the second group of stem cells comprise one or more nucleic acid sequences at least about 80% identical to a sequence selected from the group consisting of SEQ ID NO: 1 and SEQ ID NO: 14 or a fragment thereof.

12. The method of claim 1, wherein the second group of stem cells further comprise a nucleic acid sequence at least about 80% identical to SEQ ID NO: 15 or a fragment thereof.

13. The method of claim 4, wherein the third group of stem cells comprise a nucleic acid sequence at least about 80% identical to SEQ ID NO: 15 or a fragment thereof.

14.-20. (canceled)

21. A method of evaluating an agent that modulates embryogenesis, said method comprising
generating a cellular mass that comprises one or more embryonic cell structures and one or more extraembryonic cell structures by the method of claim 1; and
contacting the cellular mass with the agent.

22. A cellular mass produced by the method of claim 1, wherein the cellular mass comprises one or more embryonic cell structures and one or more extraembryonic cell structures.

23.-25. (canceled)

26. A method of generating a hematopoietic stem cell, comprising
generating a cellular mass that comprises one or more embryonic cell structures and one or more extraembryonic cell structures by the method of claim 1; and
isolating the hematopoietic stem cell from the cellular mass.

27. (canceled)

28. (canceled)

29. An in vitro or ex vivo culture system for generating a cellular mass that comprises one or more embryonic cell structures and one or more extraembryonic cell structures, said system comprising:
one or more of a first group of stem cells that comprise an inducible transgene encoding a GATA binding protein 6 (GATA6) polypeptide; and
a second group of stem cells.

30. The in vitro or ex vivo culture system of claim 29, wherein the second group of stem cells do not have the inducible transgene encoding the GATA6 polypeptide.

31. The in vitro or ex vivo culture system of claim 29, wherein the second group of stem cells comprise an inducible transgene encoding an ETS variant transcription factor 2 (ETV2) polypeptide.

32. The in vitro or ex vivo culture system of claim 29, further comprising a third group of cells, wherein the third group of cells comprise an inducible transgene encoding an ETS variant transcription factor 2 (ETV2) polypeptide.

33. The in vitro or ex vivo culture system of claim 29, wherein the first group of cells and second group of cells are induced pluripotent stem cells (iPSCs).

34. (canceled)

35. (canceled)

36. The in vitro or ex vivo culture system of claim 29, wherein the transgene encoding the GATA6 polypeptide comprises a sequence at least about 80% identical to a sequence selected from the group consisting of SEQ ID NOs: 3-10 or a fragment thereof.

37. The in vitro or ex vivo culture system of claim 29, wherein the one or more first groups of stem cells comprise a nucleic acid sequence at least about 80% identical to the sequence of SEQ ID NO: 1 or SEQ ID NO: 2 or a fragment thereof.

38. The in vitro or ex vivo culture system of claim 29, wherein the second group of stem cells comprise one or more nucleic acid sequences at least about 80% identical to a sequence selected from the group consisting of SEQ ID NO: 1 and SEQ ID NO: 14 or a fragment thereof.

39. The in vitro or ex vivo culture system of claim 29, wherein the second group of stem cells further comprise a nucleic acid sequence at least about 80% identical to SEQ ID NO: 15 or a fragment thereof.

40. The in vitro or ex vivo culture system of claim 32, wherein the third group of stem cells comprise a nucleic acid sequence at least about 80% identical to SEQ ID NO: 15 or a fragment thereof.

41. (canceled)

42. A method of generating an extracellular matrix, comprising
a) obtaining one or more of a first group of stem cells comprising an inducible transgene encoding a GATA binding protein 6 (GATA6) polypeptide;
b) contacting the one or more first groups of stem cells with an inducing agent that induces an expression of the transgene;
c) mixing the one or more first groups of stem cells of step b) with a second group of stem cells;
d) culturing the mixture of cells of step c) on a surface for at least 3 days, thereby producing a cellular mass; and
e) isolating the extracellular matrix from the cellular mass of step d).

43. An in vitro culture system comprising the extracellular matrix generated by the method of claim 42.


SEQUENCE LISTING (16 sequences)

SEQ ID NO: 1 - DNA, 4393 bp, synthetic construct
  source 1..4393: mol_type = other DNA
ctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagcaggctggatccccgggtaccggtcgccaccatgtctaggctggacaagagcaaagtcataaacggagctctggaattactcaatggtgtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgattttgaccttgacatgctccccggggaattggagggccgcggcagcctgctgacctgcggcgacgtggaggaaaaccccggccccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactcctggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccggatcgggagatgggggaggctaactgagcggccgcgaattctaccacccagctttcttgtacaaagttggcattataagaaagcattgcttatcaatttgttgcaacgaacaggtcactatcagtcaaaataaaatcattatttgccatccagctgatatcccctatagtgagtcgtattacatggtcatagctgtttcctggcagctctggcccgtgtctcaaaatctctgatgttacattgcacaagataaaataatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtt

SEQ ID NO: 2 - DNA, 13267 bp, synthetic construct
  source 1..13267: mol_type = other DNA
ggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattggagctcggtattcacgacagcaggctgaataataaaaaaattagaaactattatttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttaaacattctctcttttacaaaaataaacttattttgtactttaaaaacagtcatgttgtattataaaataagtaattagcttaacctatacataatagaaacaaattatacttattagtcagtcagaaacaactttggcacatatcaatattatgctctcgttaattaactcgagggatccaagctggccgcacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtaccccctatataagcagagctcgtttagtgaaccgtcagatcgcctggagacgccatccacgctgttttgacctccatagaagacaccgggaccgatccagcctccgcggtggcggccatcacaagtttgtacaaaaaagcaggctaggttcgccaccatggccttgactgacggcggctggtgcttgccgaagcgcttcggggccgcgggtgcggacgccagcgactccagagcctttccagcgcgggagccctccacgccgccttcccccatctcttcctcgtcctcctcctgctcccggggcggagagcggggccccggcggcgccagcaactgcgggacgcctcagctcgacacggaggcggcggccggacccccggcccgctcgctgctgctcagttcctacgcttcgcatcccttcggggctccccacggaccttcggcgcctggggtcgcgggccccgggggcaacctgtcgagctgggaggacttgctgctgttcactgacctcgaccaagccgcgaccgccagcaagctgctgtggtccagccgcggcgccaagctgagccccttcgcacccgagcagccggaggagatgtaccagaccctcgccgctctctccagccagggtccggccgcctacgacggcgcgcccggcggcttcgtgcactctgcggccgcggcggcagcagccgcggcggcggccagctccccggtctacgtgcccaccacccgcgtgggttccatgctgcccggcctaccgtaccacctgcaggggtcgggcagtgggccagccaaccacgcgggcggcgcgggcgcgcaccccggctggcctcaggcctcggccgacagccctccatacggcagcggaggcggcgcggctggcggcggggccgcggggcctggcggcgctggctcagccgcggcgcacgtctcggcgcgcttcccctactctcccagcccgcccatggccaacggcgccgcgcgggagccgggaggctacgcggcggcgggcagtgggggcgcgggaggcgtgagcggcggcggcagtagcctggcggccatgggcggccgcgagccccagtacagctcgctgtcggccgcgcggccgctgaacgggacgtaccaccaccaccaccaccaccaccaccaccatccgagcccctactcgccctacgtgggggcgccactgacgcctgcctggcccgccggacccttcgagaccccggtgctgcacagcctgcagagccgcgccggagccccgctcccggtgccccggggtcccagtgcagacctgctggaggacctgtccgagagccgcgagtgcgtgaactgcggctccatccagacgccgctgtggcggcgggacggcaccggccactacctgtgcaacgcctgcgggctctacagcaagatgaacggcctcagccggcccctcatcaagccgcagaagcgcgtgccttcatcacggcggcttggattgtcctgtgccaactgtcacaccacaactaccaccttatggcgcagaaacgccgagggtgaacccgtgtgcaatgcttgtggactctacatgaaactccatggggtgcccagaccacttgctatgaaaaaagagggaattcaaaccaggaaacgaaaacctaagaacataaataaatcaaagacttgctctggtaatagcaataattccattcccatgactccaacttccacctcttctaactcagatgattgcagcaaaaatacttcccccacaacacaacctacagcctcaggggcgggtgccccggtgatgactggtgcgggagagagcaccaatcccgagaacagcgagctcaagtattcgggtcaagatgggctctacataggcgtcagtctcgcctcgccggccgaagtcacgtcctccgtgcgaccggattcctggtgcgccctggccctggccggatctggcgccaccaacttctctctgctgaagcaggccggcgacgtggaggagaacccaggcccaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagcttacccagctttcttgtacaaagtggtgatctagctaaaacgcggcctcgaatcgaattccgcccccccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcaggaattcgattacttaagcctaggatccagatctgtacattcgaatcgatgaattcgttaaccatggaagcttctgcagcggccgcactagcggtggcggcctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgatgatgcggttttggcagtacatcaatgggcgtggatagcggtttgactcacggggatttccaagtctccaccccattgacgtcaatgggagtttgttttgactagtggagccgagagtaattcatacaaaaggagggatcgccttcgcaaggggagagcccagggaccgtccctaaattctcacagacccaaatccctgtagccgccccacgacagcgcgaggagcatgcgcccagggctgagcgcgggtagatcagagcacacaagctcacagtccccggcggtggggggaggggcgcgctgagcgggggccagggagctggcgcggggcaaactgggaaagtggtgtcgtgtgctggctccgccctcttcccgagggtgggggagaacggtatataagtgcggtagtcgccttggacgttctttttcgcaacgggtttgccgtcagaacgcaggtgagtggcgggtgtggcttccgcgggccccggagctggagccctgctctgagcgggccgggctgatatgcgagtgtcgtccgcagggtttagctgtgagcattcccacttcgagtggcgggcggtgcgggggtgagagtgcgaggcctagcggcaaccccgtagcctcgcctcgtgtccggcttgaggcctagcgtggtgtccgccgccgcgtgccactccggccgcactatgcgttttttgtccttgctgccctcgattgccttccagcagcatgggctaacaaagggagggtgtggggctcactcttaaggagcccatgaagcttacgttggataggaatggaagggcaggaggggcgactggggcccgcccgccttcggagcacatgtccgacgccacctggatggggcgaggcctgtggctttccgaagcaatcgggcgtgagtttagcctacctgggccatgtggccctagcactgggcacggtctggcctggcggtgccgcgttcccttgcctcccaacaagggtgaggccgtcccgcccggcaccagttgcttgcgcggaaagatggccgctcccggggccctgttgcaaggagctcaaaatggaggacgcggcagcccggtggagcgggcgggtgagtcacccacacaaaggaagagggccttgcccctcgccggccgctgcttcctgtgaccccgtggtctatcggccgcatagtcacctcgggcttctcttgagcaccgctcgtcgcggcggggggaggggatctaatggcgttggagtttgttcacatttggtgggtggagactagtcaggccagcctggcgctggaagtcattcttggaatttgcccctttgagtttggagcgaggctaattctcaagcctcttagcggttcaaaggtattttctaaacccgtttccaggtgttgtgaaagccaccgctaattcaaagcaatccggagtaaattcaccatgtctagactggacaagagcaaagtcataaacggcgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtaaaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatgaccgagtacaagcccacggtgcgcctcgccacccgcgacgacgtcccccgggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatgcatcgaaatgaccgaccaagcgaattcgctaggattatccctaatacctgccaccccactcttaatcagtggtggaagaacggtctcagaactgtttgtttcaattggccatttaagtttagtagtaaaagactggttaatgataacaatgcatcgtaaaaccttcagaaggaaaggagaatgttttgtggaccactttggttttcttttttgcgtgtggcagttttaagttattagtttttaaaatcagtactttttaatggaaacaacttgaccaaaaatttgtcacagaattttgagacccattaaaaaagttaaatgagaaacctgtgtgttcctttggtcaacaccgagacatttaggtgaaagacatctaattctggttttacgaatctggaaacttcttgaaaatgtaattcttgagttaacacttctgggtggagaatagggttgttttccccccacataattggaaggggaaggaatatcatttaaagctatgggagggttgctttgattacaacactggagagaaatgcagcatgttgctgattgcctgtcactaaaacaggccaaaaactgagtccttgggttgcatagaaagctgccaaacgctagccaacaagctcgtcatcgctttgcagaagagcagagaggatatgctcatcgtctaaagaactacccattttattatatattagtcacgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaaaaaagatttgcgctttactcgacctaaactttaaacacgtcatagaatcttcgtttgacaaaaaccacattgtggccaagctgtgtgacgcgacgcgcgctaaagaatggcaaaccaagtcgcgcgaggtacccagcttttgttccctttagtgagggttaattccgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 3 - DNA, 1057 bp, synthetic construct
  source 1..1057: mol_type = other DNA
atggccttgactgacggcggctggtgcttgccgaagcgcttcggggccgcgggtgcggacgccagcgactccagagcctttccagcgcgggagccctccacgccgccttcccccatctcttcctcgtcctcctcctgctcccggggcggagagcggggccccggcggcgccagcaactgcgggacgcctcagctcgacacggaggcggcggccggacccccggcccgctcgctgctgctcagttcctacgcttcgcatcccttcggggctccccacggaccttcggcgcctggggtcgcgggccccgggggcaacctgtcgagctgggaggacttgctgctgttcactgacctcgaccaagccgcgaccgccagcaagctgctgtggtccagccgcggcgccaagctgagccccttcgcacccgagcagccggaggagatgtaccagaccctcgccgctctctccagccagggtccggccgcctacgacggcgcgcccggcggcttcgtgcactctgcggccgcggcggcagcagccgcggcggcggccagctccccggtctacgtgcccaccacccgcgtgggttccatgctgcccggcctaccgtaccacctgcaggggtcgggcagtgggccagccaaccacgcgggcggcgcgggcgcgcaccccggctggcctcaggcctcggccgacagccctccatacggcagcggaggcggcgcggctggcggcggggccgcggggcctggcggcgctggctcagccgcggcgcacgtctcggcgcgcttcccctactctcccagcccgcccatggccaacggcgccgcgcgggagccgggaggctacgcggcggcgggcagtgggggcgcgggaggcgtgagcggcggcggcagtagcctggcggccatgggcggccgcgagccccagtacagctcgctgtcggccgcgcggccgctgaacgggacgtaccaccaccaccaccaccaccaccaccaccatccgagcccctactcgccctacgtgggggcgccactgacgcctgcctggcccgccggac

SEQ ID NO: 4 - DNA, 74 bp, synthetic construct
  source 1..74: mol_type = other DNA
ccttcgagaccccggtgctgcacagcctgcagagccgcgccggagccccgctcccggtgccccggggtcccagt

SEQ ID NO: 5 - DNA, 164 bp, synthetic construct
  source 1..164: mol_type = other DNA
gacctgctggaggacctgtccgagagccgcgagtgcgtgaactgcggctccatccagacgccgctgtggcggcgggacggcaccggccactacctgtgcaacgcctgcgggctctacagcaagatgaacggcctcagccggcccctcatcaagccgcagaagcg

SEQ ID NO: 6 - DNA, 122 bp, synthetic construct
  source 1..122: mol_type = other DNA
ccttcatcacggcggcttggattgtcctgtgccaactgtcacaccacaactaccaccttatggcgcagaaacgccgagggtgaacccgtgtgcaatgcttgtggactctacatgaaactcca

SEQ ID NO: 7 - DNA, 84 bp, synthetic construct
  source 1..84: mol_type = other DNA
gtgcccagaccacttgctatgaaaaaagagggaattcaaaccaggaaacgaaaacctaagaacataaataaatcaaagacttgc

SEQ ID NO: 8 - DNA, 99 bp, synthetic construct
  source 1..99: mol_type = other DNA
gtaatagcaataattccattcccatgactccaacttccacctcttctaactcagatgattgcagcaaaaatacttcccccacaacacaacctacagcct

SEQ ID NO: 9 - DNA, 165 bp, synthetic construct
  source 1..165: mol_type = other DNA
gcgggtgccccggtgatgactggtgcgggagagagcaccaatcccgagaacagcgagctcaagtattcgggtcaagatgggctctacataggcgtcagtctcgcctcgccggccgaagtcacgtcctccgtgcgaccggattcctggtgcgccctggccctggcc

SEQ ID NO: 10 - DNA, 1785 bp, synthetic construct
  source 1..1785: mol_type = other DNA
atggccttgactgacggcggctggtgcttgccgaagcgcttcggggccgcgggtgcggacgccagcgactccagagcctttccagcgcgggagccctccacgccgccttcccccatctcttcctcgtcctcctcctgctcccggggcggagagcggggccccggcggcgccagcaactgcgggacgcctcagctcgacacggaggcggcggccggacccccggcccgctcgctgctgctcagttcctacgcttcgcatcccttcggggctccccacggaccttcggcgcctggggtcgcgggccccgggggcaacctgtcgagctgggaggacttgctgctgttcactgacctcgaccaagccgcgaccgccagcaagctgctgtggtccagccgcggcgccaagctgagccccttcgcacccgagcagccggaggagatgtaccagaccctcgccgctctctccagccagggtccggccgcctacgacggcgcgcccggcggcttcgtgcactctgcggccgcggcggcagcagccgcggcggcggccagctccccggtctacgtgcccaccacccgcgtgggttccatgctgcccggcctaccgtaccacctgcaggggtcgggcagtgggccagccaaccacgcgggcggcgcgggcgcgcaccccggctggcctcaggcctcggccgacagccctccatacggcagcggaggcggcgcggctggcggcggggccgcggggcctggcggcgctggctcagccgcggcgcacgtctcggcgcgcttcccctactctcccagcccgcccatggccaacggcgccgcgcgggagccgggaggctacgcggcggcgggcagtgggggcgcgggaggcgtgagcggcggcggcagtagcctggcggccatgggcggccgcgagccccagtacagctcgctgtcggccgcgcggccgctgaacgggacgtaccaccaccaccaccaccaccaccaccaccatccgagcccctactcgccctacgtgggggcgccactgacgcctgcctggcccgccggacccttcgagaccccggtgctgcacagcctgcagagccgcgccggagccccgctcccggtgccccggggtcccagtgcagacctgctggaggacctgtccgagagccgcgagtgcgtgaactgcggctccatccagacgccgctgtggcggcgggacggcaccggccactacctgtgcaacgcctgcgggctctacagcaagatgaacggcctcagccggcccctcatcaagccgcagaagcgcgtgccttcatcacggcggcttggattgtcctgtgccaactgtcacaccacaactaccaccttatggcgcagaaacgccgagggtgaacccgtgtgcaatgcttgtggactctacatgaaactccatggggtgcccagaccacttgctatgaaaaaagagggaattcaaaccaggaaacgaaaacctaagaacataaataaatcaaagacttgctctggtaatagcaataattccattcccatgactccaacttccacctcttctaactcagatgattgcagcaaaaatacttcccccacaacacaacctacagcctcaggggcgggtgccccggtgatgactggtgcgggagagagcaccaatcccgagaacagcgagctcaagtattcgggtcaagatgggctctacataggcgtcagtctcgcctcgccggccgaagtcacgtcctccgtgcgaccggattcctggtgcgccctggccctggcc

SEQ ID NO: 11 - DNA, 19 bp, synthetic construct
  source 1..19: mol_type = other DNA
tccctatcagtgatagaga

SEQ ID NO: 12 - protein, 360 aa, synthetic construct
MAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 13 - protein, 442 aa, synthetic construct
MYQSLAMAANHGPPPGAYEAGGPGAFMHGAGAASSPVYVPTPRVPSSVLGLSYLQGGGAGSASGGASGGSSGGAASGAGPGTQQGSPGWSQAGADGAAYTPPPVSPRFSFPGTTGSLAAAAAAAAAREAAAYSSGGGAAGAGLAGREQYGRAGFAGSYSSPYPAYMADVGASWAAAAAASAGPFDSPVLHSLPGRANPAARHPNLDMFDDFSEGRECVNCGAMSTPLWRRDGTGHYLCNACGLYHKMNGINRPLIKPQRRLSASRRVGLSCANCQTTTTTLWRRNAEGEPVCNACGLYMKLHGVPRPLAMRKEGIQTRKRKPKNLNKSKTPAAPSGSESLPPASGASSNSSNATTSSSEEMRPIKTEPGLSSHYGHSSSVSQTFSVSAMSGHGPSIHPVLSALKLSPQGYASPVSQSPQTSSKQDSWNSLVLADSHGDIITA

SEQ ID NO: 14 - DNA, 11846 bp, synthetic construct
  source 1..11846: mol_type = other DNA
ggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattggagctcggtattcacgacagcaggctgaataataaaaaaattagaaactattatttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttaaacattctctcttttacaaaaataaacttattttgtactttaaaaacagtcatgttgtattataaaataagtaattagcttaacctatacataatagaaacaaattatacttattagtcagtcagaaacaactttggcacatatcaatattatgctctcgttaattaactcgagggatccaagctggccgcacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtaccccctatataagcagagctcgtttagtgaaccgtcagatcgcctggagacgccatccacgctgttttgacctccatagaagacaccgggaccgatccagcctccgcggtggcggccatcacaagtttgtacaaaaaagcaggctgattaccggttgccaccgtacaaaaaagcagaagggccgtcaaggcccaccatggacctgtggaactgggatgaggcatccccacaggaagtgcctccagggaacaagctggcagggcttgaaggagccaaattaggcttctgtttccctgatctggcactccaaggggacacgccgacagcgacagcagagacatgctggaaaggtacaagctcatccctggcaagcttcccacagctggactggggctccgcgttactgcacccagaagttccatggggggcggagcccgactctcaggctcttccgtggtccggggactggacagacatggcgtgcacagcctgggactcttggagcggcgcctcgcagaccctgggccccgcccctctcggcccgggccccatccccgccgccggctccgaaggcgccgcgggccagaactgcgtccccgtggcgggagaggccacctcgtggtcgcgcgcccaggccgccgggagcaacaccagctgggactgttctgtggggcccgacggcgatacctactggggcagtggcctgggcggggagccgcgcacggactgtaccatttcgtggggcgggcccgcgggcccggactgtaccacctcctggaacccggggctgcatgcgggtggcaccacctctttgaagcggtaccagagctcagctctcaccgtttgctccgaaccgagcccgcagtcggaccgtgccagtttggctcgatgccccaaaactaaccaccgaggtcccattcagctgtggcagttcctcctggagctgctccacgacggggcgcgtagcagctgcatccgttggactggcaacagccgcgagttccagctgtgcgaccccaaagaggtggctcggctgtggggcgagcgcaagagaaagccgggcatgaattacgagaagctgagccggggccttcgctactactatcgccgcgacatcgtgcgcaagagcggggggcgaaagtacacgtaccgcttcgggggccgcgtgcccagcctagcctatccggactgtgcgggaggcggacggggagcagagacacaatagggcctcatgggcccagctttcttgtacgaattctgcagtcgacggtaccgcgggcccgggatccaccggatctagataactgatctacccagctttcttgtacaaagtggtgatctagctaaaacgcggcctcgaatcgaattccgcccccccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcaggaattcgattacttaagcctaggatccagatctgtacattcgaatcgatgaattcgttaaccatggaagcttctgcagcggccgcactagcggtggcggcctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgatgatgcggttttggcagtacatcaatgggcgtggatagcggtttgactcacggggatttccaagtctccaccccattgacgtcaatgggagtttgttttgactagtggagccgagagtaattcatacaaaaggagggatcgccttcgcaaggggagagcccagggaccgtccctaaattctcacagacccaaatccctgtagccgccccacgacagcgcgaggagcatgcgcccagggctgagcgcgggtagatcagagcacacaagctcacagtccccggcggtggggggaggggcgcgctgagcgggggccagggagctggcgcggggcaaactgggaaagtggtgtcgtgtgctggctccgccctcttcccgagggtgggggagaacggtatataagtgcggtagtcgccttggacgttctttttcgcaacgggtttgccgtcagaacgcaggtgagtggcgggtgtggcttccgcgggccccggagctggagccctgctctgagcgggccgggctgatatgcgagtgtcgtccgcagggtttagctgtgagcattcccacttcgagtggcgggcggtgcgggggtgagagtgcgaggcctagcggcaaccccgtagcctcgcctcgtgtccggcttgaggcctagcgtggtgtccgccgccgcgtgccactccggccgcactatgcgttttttgtccttgctgccctcgattgccttccagcagcatgggctaacaaagggagggtgtggggctcactcttaaggagcccatgaagcttacgttggataggaatggaagggcaggaggggcgactggggcccgcccgccttcggagcacatgtccgacgccacctggatggggcgaggcctgtggctttccgaagcaatcgggcgtgagtttagcctacctgggccatgtggccctagcactgggcacggtctggcctggcggtgccgcgttcccttgcctcccaacaagggtgaggccgtcccgcccggcaccagttgcttgcgcggaaagatggccgctcccggggccctgttgcaaggagctcaaaatggaggacgcggcagcccggtggagcgggcgggtgagtcacccacacaaaggaagagggccttgcccctcgccggccgctgcttcctgtgaccccgtggtctatcggccgcatagtcacctcgggcttctcttgagcaccgctcgtcgcggcggggggaggggatctaatggcgttggagtttgttcacatttggtgggtggagactagtcaggccagcctggcgctggaagtcattcttggaatttgcccctttgagtttggagcgaggctaattctcaagcctcttagcggttcaaaggtattttctaaacccgtttccaggtgttgtgaaagccaccgctaattcaaagcaatccggagtaaattcaccatgtctagactggacaagagcaaagtcataaacggcgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtaaaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatgaccgagtacaagcccacggtgcgcctcgccacccgcgacgacgtcccccgggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatgcatcgaaatgaccgaccaagcgaattcgctaggattatccctaatacctgccaccccactcttaatcagtggtggaagaacggtctcagaactgtttgtttcaattggccatttaagtttagtagtaaaagactggttaatgataacaatgcatcgtaaaaccttcagaaggaaaggagaatgttttgtggaccactttggttttcttttttgcgtgtggcagttttaagttattagtttttaaaatcagtactttttaatggaaacaacttgaccaaaaatttgtcacagaattttgagacccattaaaaaagttaaatgagaaacctgtgtgttcctttggtcaacaccgagacatttaggtgaaagacatctaattctggttttacgaatctggaaacttcttgaaaatgtaattcttgagttaacacttctgggtggagaatagggttgttttccccccacataattggaaggggaaggaatatcatttaaagctatgggagggttgctttgattacaacactggagagaaatgcagcatgttgctgattgcctgtcactaaaacaggccaaaaactgagtccttgggttgcatagaaagctgccaaacgctagccaacaagctcgtcatcgctttgcagaagagcagagaggatatgctcatcgtctaaagaactacccattttattatatattagtcacgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaaaaaagatttgcgctttactcgacctaaactttaaacacgtcatagaatcttcgtttgacaaaaaccacattgtggccaagctgtgtgacgcgacgcgcgctaaagaatggcaaaccaagtcgcgcgaggtacccagcttttgttccctttagtgagggttaattccgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 15 - DNA, 1029 bp, synthetic construct
  source 1..1029: mol_type = other DNA
atggacctgtggaactgggatgaggcatccccacaggaagtgcctccagggaacaagctggcagggcttgaaggagccaaattaggcttctgtttccctgatctggcactccaaggggacacgccgacagcgacagcagagacatgctggaaaggtacaagctcatccctggcaagcttcccacagctggactggggctccgcgttactgcacccagaagttccatggggggcggagcccgactctcaggctcttccgtggtccggggactggacagacatggcgtgcacagcctgggactcttggagcggcgcctcgcagaccctgggccccgcccctctcggcccgggccccatccccgccgccggctccgaaggcgccgcgggccagaactgcgtccccgtggcgggagaggccacctcgtggtcgcgcgcccaggccgccgggagcaacaccagctgggactgttctgtggggcccgacggcgatacctactggggcagtggcctgggcggggagccgcgcacggactgtaccatttcgtggggcgggcccgcgggcccggactgtaccacctcctggaacccggggctgcatgcgggtggcaccacctctttgaagcggtaccagagctcagctctcaccgtttgctccgaaccgagcccgcagtcggaccgtgccagtttggctcgatgccccaaaactaaccaccgaggtcccattcagctgtggcagttcctcctggagctgctccacgacggggcgcgtagcagctgcatccgttggactggcaacagccgcgagttccagctgtgcgaccccaaagaggtggctcggctgtggggcgagcgcaagagaaagccgggcatgaattacgagaagctgagccggggccttcgctactactatcgccgcgacatcgtgcgcaagagcggggggcgaaagtacacgtaccgcttcgggggccgcgtgcccagcctagcctatccggactgtgcgggaggcggacggggagcagagacacaatag

SEQ ID NO: 16 - protein, 342 aa, synthetic construct
MDLWNWDEASPQEVPPGNKLAGLEGAKLGFCFPDLALQGDTPTATAETCWKGTSSSLASFPQLDWGSALLHPEVPWGAEPDSQALPWSGDWTDMACTAWDSWSGASQTLGPAPLGPGPIPAAGSEGAAGQNCVPVAGEATSWSRAQAAGSNTSWDCSVGPDGDTYWGSGLGGEPRTDCTISWGGPAGPDCTTSWNPGLHAGGTTSLKRYQSSALTVCSEPSPQSDRASLARCPKTNHRGPIQLWQFLLELLHDGARSSCIRWTGNSREFQLCDPKEVARLWGERKRKPGMNYEKLSRGLRYYYRRDIVRKSGGRKYTYRFGGRVPSLAYPDCAGGGRGAETQ